(19) United States
(12) Patent Application Publication
(10) Pub. No.: US 2025/0064355 A1
(43) Pub. Date: Feb. 27, 2025

(54) ANALYTE MONITORING SYSTEMS AND METHODS FOR MONITORING MULTIPLE ANALYTES

(71) Applicant: Abbott Diabetes Care Inc., Alameda, CA (US)

(72) Inventors: Udo HOSS, San Ramon, CA (US); Peter G. ROBINSON, Alamo, CA (US); Nolan R. CANNADY, Oakland, CA (US); Elaine LOVELADY, Witney (GB); Sadie SINCLAIR, Uxbridge (GB); Simon Andrew HECTOR, Swindon (GB)

(21) Appl. No.: 18/813,683

(22) Filed: Aug. 23, 2024

Related U.S. Application Data
(60) Provisional application No. 63/596,459, filed on Nov. 6, 2023.
(60) Provisional application No. 63/588,887, filed on Oct. 9, 2023.
(60) Provisional application No. 63/578,872, filed on Aug. 25, 2023.

(51) Int. Cl. A61B 5/145; A61B 5/00

(52) U.S. Cl. CPC A61B 5/14546; A61B 5/6801

(57) ABSTRACT

An analyte monitoring assembly for monitoring one or more analytes in a bodily fluid of a user includes a housing configured to be mounted on a body of a user and sensor electronics arranged within the housing. An analyte sensor includes a body portion coupled to the sensor electronics and arranged within the housing and one or more tail portions configured to be positioned in the body of the user. The analyte sensor further includes a first electrode comprising a first active area for detecting a first analyte, and a second electrode comprising a second active area for detecting a second analyte.

### CROSS REFERENCE TO RELATED APPLICATIONS

This application claims priority to and the benefit of U.S. Provisional Application No. 63/578,872, filed Aug. 25, 2023, U.S. Provisional Application No. 63/588,887, filed Oct. 9, 2023, and U.S. Provisional Application No. 63/596,459, filed Nov. 6, 2023, the disclosures of each of which are incorporated herein by reference in their entireties.

### FIELD

This disclosure generally relates to analyte sensors and assemblies, including in vivo analyte sensors for sensing and monitoring analytes in a bodily fluid, and analyte sensors and assemblies for detecting multiple analytes. Specifically, embodiments described herein relate to analyte sensors having a substrate layer arranged in a tubular configuration for detecting multiple analytes in a bodily fluid of a user, and/or analyte monitoring assemblies having an analyte sensor with a plurality of sensor tails for detecting a plurality of analytes in a bodily fluid, and/or an in vivo analyte sensor having a reduced profile sensor tail for insertion into a body of a user.

### BACKGROUND

Many individuals monitor the levels of one or more analytes in a bodily fluid to assess their health or to monitor a disease state. For example, diabetic patients may monitor glucose levels to assess their state of glycemia and to avoid hyperglycemia and hypoglycemia. Analyte monitoring devices may be used to monitor analyte levels in a bodily fluid of an individual on a continuous or periodic basis. Such monitoring devices may process analyte data and output information to the individual and to caregivers and healthcare professionals. The analyte data may be useful to inform the individual of the impact of various activities on their analyte levels, such as meals, exercise, or medication, among others. The analyte data may also be shared with caregivers and healthcare professionals to help determine or modify treatment plans, such to help determine amounts and types of medication to administer, and behavior or lifestyle changes.

Analyte sensors have been developed to facilitate long-term monitoring of bodily fluid analytes, such as glucose. Analyte sensors typically include a sensor tail positioned below the skin surface, e.g., in the subcutaneous or dermal tissue, and having sensing chemistry for detecting analytes in the bodily fluids. Monitoring the level of one or more analytes in the bodily fluid may help the user to track a condition or disease, or to monitor their health and wellness. Based on analyte levels, the user may modify self-care behaviors, such as by modifying their diet and exercise, or may adjust treatment regimens, such as by modifying dosages or timing of medication. For users with diseases, such as diabetes, monitoring analyte levels may allow the user to avoid or minimize negative health effects, such as hypoglycemia or hyperglycemia by identifying a pattern of low or high glucose levels and taking corrective action.

### SUMMARY OF THE INVENTION

Some embodiments described herein relate to an analyte monitoring assembly for monitoring one or more analytes in a bodily fluid of a user. The analyte monitoring assembly includes a housing configured to be mounted on a body of a user, sensor electronics arranged within the housing, and an analyte sensor. The analyte sensor includes a body portion coupled to the sensor electronics and arranged within the housing, and one or more tail portions configured to be positioned in the body of the user. The analyte sensor further includes a first electrode having a first active area for detecting a first analyte, and a second electrode having a second active area for detecting a second analyte.

In any of the various embodiments described herein, the first electrode may be connected to a first electrical contact on the body portion and the second electrode may be connected to a second electrical contact on the body portion.

In any of the various embodiments described herein, each of the one or more tail portions includes a longitudinal axis extending from a proximal end of the tail portion to a distal end opposite the proximal end, wherein the first electrode comprises a first side opposite a second side in a direction traverse to the longitudinal axis, wherein a width of the first electrode measured from the first side to the second side is in a range of 150 microns to 250 microns. In some embodiments, a ratio of the width of the first electrode to a width of the first active area may be in a range of 1:1 to 1.7:1. In some embodiments, the width of the first electrode may be the same as a width of the first active area.

In any of the various embodiments described herein, the first active area may include one or more spots of a reagent composition.

In any of the various embodiments described herein, the one or more tail portions include a first tail portion including the first electrode and the first active area and a second tail portion including the second electrode and the second active area. In some embodiments, the first tail portion includes a single electrode.

In any of the various embodiments described herein, a tail portion of the one or more tail portions of the analyte sensor comprises a tubular configuration. In some embodiments, the tail portion having the tubular configuration includes the first electrode, the first active area, the second electrode, and the second active area.

Some embodiments described herein relate to an analyte monitoring assembly for monitoring one or more analytes in a bodily fluid of a user. The analyte monitoring assembly includes a housing configured to be mounted on a body of the user, sensor electronics arranged within the housing, and an analyte sensor. The analyte sensor includes a body portion coupled to the sensor electronics and arranged within the housing, and a plurality of tail portions extending from the body portion. Each of the plurality of body portions may be configured to extend out of the housing and into the body of the user to detect the one or more analytes in the bodily fluid of the user. A first tail portion of the plurality of tail portions includes a first active area for detecting signals indicative of a first analyte in the bodily fluid, and a second tail portion of the plurality of tail portions includes a second active area for detecting signals indicative of a second analyte in the bodily fluid.

In any of the various embodiments described herein, the first tail portion may have a longitudinal axis, a first side opposite a second side, and a first surface opposite a second surface, wherein a width of the first tail portion is measured from the first side to the opposing second side in a direction transverse to the longitudinal axis, and the width may be in a range of 150 μm to 250 μm. In some embodiments, a ratio of the width of the first tail portion to a width of the first active area may be in a range of 1:1 to 1.7:1. In some embodiments, the width of the first tail portion may be the same as a width of the first active area. In some embodiments, the first active area may include one or more spots of a reagent composition.

Some embodiments described herein relate to an analyte monitoring assembly for monitoring multiple analytes in a bodily fluid of a user. The analyte monitoring assembly includes a housing configured to be worn on a body of a user, sensor electronics arranged within the housing, and an analyte sensor. The analyte sensor includes a substrate layer having a body portion configured to be arranged above a skin surface of the user, and a tail portion configured to be arranged below the skin surface and in contact with the bodily fluid of the user for sensing analytes in the bodily fluid. The tail portion includes a tubular configuration. A first electrode is disposed on an exterior surface of the tail portion of the substrate layer, and a first active area is disposed on the first electrode for detecting a first analyte. A second electrode is disposed on the exterior surface of the tail portion of the substrate layer, and a second active area is disposed on the second electrode for detecting a second analyte, wherein the second analyte is different from the first analyte.

In any of the various embodiments described herein, the tail portion may have a longitudinal axis, wherein the first electrode has a first side opposite a second side, and a width of the first electrode measured from the first side to the opposing second side in a direction transverse to the longitudinal axis may be in a range of 150 μm to 250 μm. In some embodiments, a ratio of the width of the first electrode to a width of the first active area may be in a range of 1:1 to 1.7:1. In some embodiments, the width of the first electrode may be the same as a width of the first active area. In some embodiments, the first active area may include one or more spots of a reagent composition.

Some embodiments described herein relate to an in vivo analyte sensor for monitoring multiple analytes in a bodily fluid of a user. The analyte sensor includes a substrate layer arranged in a tubular configuration and having a first portion, also known as a body or body portion, configured to be arranged above the skin surface of the user, and a second portion, also known as a tail or tail portion, configured to be arranged below the skin surface of the user and in contact with the bodily fluid of the user for sensing analytes in the bodily fluid. The analyte sensor further includes a first electrode disposed on an exterior surface of the second portion of the substrate layer and a first reagent disposed on the first electrode for detecting a first analyte. The analyte sensor includes a second electrode disposed on the exterior surface of the second portion of the substrate layer and a second reagent disposed on the second electrode for detecting a second analyte, wherein the second analyte is different than the first analyte.

In any of the various embodiments described herein, the first electrode may include a conductive material disposed on the substrate layer.

In any of the various embodiments described herein, the first electrode may be electrically isolated from the second electrode.

In any of the various embodiments described herein, the substrate layer may include a non-conductive material.

In any of the various embodiments described herein, the first reagent includes one or more spots disposed on the first electrode.

In any of the various embodiments described herein, the first electrode may be electrically connected to a first electrical contact, wherein the first electrical contact is configured to be placed in communication with sensor electronics.

In any of the various embodiments described herein, the first and second electrodes may be spaced from one another in a circumferential direction of the substrate layer.

In any of the various embodiments described herein, the first and second electrodes may be spaced from one another in a longitudinal direction of the substrate layer.

In any of the various embodiments described herein, one or more membranes may be disposed on the first electrode and the first reagent.

In any of the various embodiments described herein, the substrate layer may include a core that is arranged in contact with an interior surface of the substrate layer when the substrate layer is arranged in the tubular configuration.

Some embodiments described herein relate to an analyte monitoring device for detecting multiple analytes and that includes a housing configured to be arranged on a skin surface of a user. The analyte monitoring device further includes sensor electronics coupled to the housing and having one or more processors, a memory coupled to the one or more processors, and wireless communication circuitry for communicating analyte data to a receiver. The analyte monitoring device includes an in vivo analyte sensor having a substrate layer arranged in a tubular configuration, wherein the substrate layer comprises a first portion, also known as a body or body portion, coupled to the sensor electronics and configured to be arranged above the skin surface of the user, and a second portion, also known as a tail or tail portion, configured to be arranged below the skin surface of the user and in contact with the bodily fluid of the user for sensing analytes in the bodily fluid. The in vivo analyte sensor includes a first electrode disposed on an exterior surface of the second portion of the substrate layer, and a first reagent disposed on the first electrode for detecting a first analyte. The in vivo analyte sensor further includes a second electrode disposed on the exterior surface of the second portion of the substrate layer, and a second reagent disposed on the second electrode for detecting a second analyte, wherein the second analyte is different from the first analyte.

In any of the various embodiments described herein, the first electrode may include a conductive material disposed on the substrate layer.

In any of the various embodiments described herein, the first electrode may be electrically isolated from the second electrode.

In any of the various embodiments described herein, the substrate layer may include a non-conductive material.

In any of the various embodiments described herein, a plurality of spots of the first reagent may be disposed on the first electrode.

In any of the various embodiments described herein, the first electrode may be electrically connected to a first electrical contact, wherein the first electrical contact is configured to be placed in communication with sensor electronics.

In any of the various embodiments described herein, the first and second electrodes may be spaced from one another in a circumferential direction of the substrate layer.

In any of the various embodiments described herein, the first and second electrodes may be spaced from one another in a longitudinal direction of the substrate layer.

In any of the various embodiments described herein, the in vivo analyte sensor may include one or more membranes disposed on the first electrode and the first reagent.

In any of the various embodiments described herein, the substrate layer may include a core that is arranged in contact with an interior surface of the substrate layer when the substrate layer is in the tubular configuration.

Some embodiments described herein relate to a method of manufacturing an analyte sensor for monitoring multiple analytes in a bodily fluid. The method includes forming a first electrode on a substrate layer and forming a second electrode on the substrate layer. The method further includes depositing a first reagent on the first electrode, depositing a second reagent on the second electrode, and arranging the substrate layer in a tubular configuration.

In any of the various embodiments described herein, forming the first electrode may include depositing a conductive material on the substrate layer.

In any of the various embodiments described herein, the method may further include encapsulating the first electrode and the first reagent in a membrane.

In any of the various embodiments described herein, the method may further include arranging the first electrode on the substrate layer such that the first electrode is spaced from the second electrode in a circumferential direction of the analyte sensor.

In any of the various embodiments described herein, the method may further include arranging the first electrode on the substrate layer such that the first electrode is spaced from the second electrode in a longitudinal direction of the analyte sensor.

In any of the various embodiments described herein, the method may further include forming a first electrical contact on a first extension of the substrate layer, wherein the first electrical contact is in electrical communication with the first electrode, and forming a second electrical contact on a second extension of the substrate layer, wherein the second electrical contact is in electrical communication with the second electrode. In some embodiments, the method may further include folding the first extension and the second extension such that the first extension and the second extension are arranged in a plane that is transverse to a longitudinal axis of the substrate layer in the tubular configuration.

Some embodiments described herein relate to an analyte monitoring assembly for monitoring one or more analytes in a bodily fluid of a user, and that includes a housing having a base configured to be mounted to a body of a user. The analyte monitoring assembly includes an analyte sensor including a body and a plurality of sensor tails extending from the body, wherein the body is arranged on the housing and the plurality of sensor tails is configured to extend out of the housing and into the body of the user to detect the one or more analytes in the bodily fluid of the user. A first sensor tail of the plurality of sensor tails includes a first active area for detecting signals indicative of a first analyte in the bodily fluid, and a second sensor tail of the plurality of sensor tails includes a second active area for detecting signals indicative of a second analyte in the bodily fluid. The analyte monitoring assembly further includes sensor electronics arranged within the housing and coupled to the analyte sensor, wherein the sensor electronics is configured to receive the signals indicative of the first and second analytes from the analyte sensor and to wirelessly transmit analyte data to a receiver device.

In any of the various embodiments described herein, the body of the analyte sensor may be arranged perpendicularly to at least one of the plurality of sensor tails.

In any of the various embodiments described herein, the body of the analyte sensor may be arranged coplanar with at least one of the plurality of sensor tails.

In any of the various embodiments described herein, the body of the analyte sensor may have a planar configuration.

In any of the various embodiments described herein, the body of the analyte sensor may have a curvature.

In any of the various embodiments described herein, the housing may include one or more openings, optionally a plurality of openings, through which the plurality of sensor tails extend. In some embodiments, the plurality of openings may be arranged linearly on the base of the housing. In some embodiments, the plurality of openings may be arranged in a ring shape on the base of the housing.

In any of the various embodiments described herein, each of the plurality of sensor tails may have a length in a range of 1 mm to 4 mm, optionally a length greater than 1 mm or a length less than 4 mm. In some embodiments, each of the plurality of sensor tails may have the same length.

In any of the various embodiments described herein, a maximum thickness of each of the plurality of sensor tails may be in a range of 150 microns to 400 microns, optionally a maximum thickness of greater than 150 microns or a maximum thickness of less than 400 microns.

In any of the various embodiments described herein, the sensor electronics may be removably coupled to the housing.

In any of the various embodiments described herein, each of the plurality of sensor tails may include a single working electrode and an analyte-responsive enzyme.

Some embodiments described herein relate to a system for monitoring a plurality of analytes in a bodily fluid of a user, the system including an analyte monitoring assembly having a housing with a base configured to be mounted to a body of a user. The analyte monitoring assembly includes an analyte sensor having a body and a plurality of sensor tails extending from the body, wherein the body is arranged on the housing and the plurality of sensor tails is configured to extend out of the housing and into the body of the user to detect the one or more analytes in the bodily fluid of the user. A first sensor tail of the plurality of sensor tails includes a first active area for detecting signals indicative of a first analyte in the bodily fluid, and a second sensor tail of the plurality of sensor tails includes a second active area for detecting signals indicative of a second analyte in the bodily fluid. The analyte monitoring assembly further includes sensor electronics arranged within the housing and coupled to the analyte sensor, wherein the sensor electronics is configured to receive the signals indicative of the first and second analytes from the analyte sensor and to wirelessly transmit analyte data to a receiver device. The system further includes an insertion device including a body and a plurality of needles extending from the body, wherein the needles are configured to pierce skin of the user to facilitate insertion of the plurality of sensor tails.

The system for monitoring a plurality of analytes may include the analyte monitoring assembly of the first aspect described above.

In any of the various embodiments described herein, each of the plurality of needles may correspond to one of the plurality of sensor tails.

In any of the various embodiments described herein, each of the plurality of needles may have a length that is greater than a length of each of the plurality of sensor tails.

In any of the various embodiments described herein, the body of the insertion device may have a curvature.

In any of the various embodiments described herein, the body of the insertion device may be arranged perpendicularly to one or more of the plurality of needles.

Some embodiments described herein relate to an analyte monitoring assembly for monitoring analytes in a bodily fluid of a user, the analyte monitoring assembly includes a housing having a base configured to be mounted on a body of the user. The analyte monitoring assembly includes a first analyte sensor arranged on the housing and having a first body and a first plurality of sensor tails extending from the first body, wherein the first plurality of sensor tails are configured to detect one or more of the analytes in the bodily fluid. A second analyte sensor is arranged on the housing and includes a second body and a second plurality of sensor tails extending from the second body, wherein each of the second plurality of sensor tails are configured to detect one or more of the analytes in the bodily fluid. The analyte monitoring assembly further includes sensor electronics arranged within the housing and coupled to both the first and second analyte sensors, wherein the sensor electronics is configured to receive signals indicative of the one or more analytes from the first and second analyte sensors. The sensor electronics includes a processor, a memory coupled to the processor, and wireless communication circuitry configured to transmit analyte data to a receiver device.

In any of the various embodiments described herein, the first analyte sensor may be configured to detect a first plurality of analytes, and the second analyte sensor may be configured to detect a second plurality of analytes.

In any of the various embodiments described herein, the first plurality of sensor tails and the second plurality of sensor tails may extend through an opening on the base of the housing.

In any of the various embodiments described herein, a first sensor tail of the first plurality of sensor tails may include a working electrode, and wherein a second sensor tail of the first plurality of sensor tails may include a reference electrode or a counter electrode.

Some embodiments described herein relate to an analyte monitoring assembly configured to detect a plurality of analytes in a bodily fluid of a user, the analyte monitoring assembly including a housing configured to be worn on a body of a user. The analyte monitoring assembly further includes a plug assembly having a sensor support configured to be coupled to the housing, an analyte sensor comprising a body, a contact portion comprising one or more electrical contacts, and a plurality of sensor tails extending from the body, wherein the plurality of sensor tails are configured to detect the plurality of analytes in the bodily fluid, and wherein the analyte sensor is arranged on the sensor support. The plug assembly further includes an insertion device having a body and a plurality of needles extending from the body, wherein the insertion device is removably secured to the plug assembly. The analyte monitoring assembly includes sensor electronics in electrical communication with the analyte sensor to receive signals indicative of analyte levels from the analyte sensor.

In any of the various embodiments described herein, each of the plurality of needles of the insertion device may correspond to a sensor tail of the plurality of sensor tails.

In any of the various embodiments described herein, the insertion device may further include a plurality of extensions extending from the body of the insertion device.

In any of the various embodiments described herein, the contact portion may be arranged perpendicularly to the plurality of sensor tails. In some embodiments, the sensor support may include a body defining a central opening, a platform arranged within the central opening, and a channel defined between the body and the platform, wherein the body of the analyte sensor is arranged within the channel, and the contact portion is arranged on the platform.

In any of the various embodiments described herein, the analyte monitoring assembly may include a connector having a body with a plurality of electrical contacts in electrical communication with electrical contacts of the analyte sensor.

The housing as described in any of the aspects above may be mounted to the user directly, for example with a strap or over-patch, or via interface elements such as one or more adhesive patches. An analyte monitoring assembly as described herein may comprise one or more analyte sensors having one or more bodies, wherein each sensor tail extends from a body of an analyte sensor. The first analyte may be the same or different to the second analyte. Moreover, in some embodiments, the signals may be transmitted via a wired or wireless connection to a receiver device.

Some embodiments described herein relate to an in vivo analyte sensor for monitoring an analyte in a bodily fluid of a user. The in vivo analyte sensor includes a portion configured to be inserted under a skin surface, also referred to as a sensor tail, that includes a substrate, a working electrode disposed on the substrate, and an active area disposed on the working electrode for detecting the analyte in the bodily fluid of the user. The sensor tail has a longitudinal axis, a first side opposite a second side, and a first surface opposite a second surface. A width of the sensor tail is measured from the first side to the opposing second side of the sensor tail in a direction transverse to the longitudinal axis of the sensor tail, and the width of the sensor tail is in a range of 150 μm to 250 μm. The analyte sensor further includes a flag, also referred to as a body or body portion, including an electrical contact electrically connected to the working electrode and a neck connecting the sensor tail and the flag.

In any of the various embodiments described herein, the width of the sensor tail may be in a range of 170 μm to 230 μm.

In any of the various embodiments described herein, the width of the sensor tail may be the same as a width of the active area.

In any of the various embodiments described herein, a ratio of the width of the sensor tail to a width of the active area is in a range of 1:1 to 1.7:1

In any of the various embodiments described herein, the substrate and the working electrode may each have a first side opposite a second side and a first surface opposite a second surface extending across at least a part of the width of the sensor tail, and the active area may be disposed on a surface of the working electrode.

In any of the various embodiments described herein, a distance from the first side and/or the second side of the sensor tail to an edge of the active area, and/or a distance from a first and/or a second side of the substrate to an edge of the active area, may be in a range of 10 μm to 90 μm.

In any of the various embodiments described herein, the active area may include an analyte-responsive enzyme.

In any of the various embodiments described herein, the active area may include a plurality of spots of a reagent composition.

In any of the various embodiments described herein, the sensor tail may further include a membrane covering at least the active area.

In any of the various embodiments described herein, the flag may be arranged perpendicularly to the sensor tail.

In any of the various embodiments described herein, the sensor tail may further include a counter electrode or a reference electrode.

In any of the various embodiments described herein, the sensor tail may include a rounded tip.

Some embodiments described herein relate to a sensor control device for monitoring an analyte level in a bodily fluid of a user. The sensor control device includes an electronics housing, sensor electronics arranged within the electronics housing, and an analyte sensor coupled with the sensor electronics and configured to measure an analyte level. The analyte sensor includes a sensor tail having a substrate, a working electrode disposed on the substrate, and an active area disposed on the working electrode for detecting the analyte in the bodily fluid of the user. The sensor tail has a longitudinal axis, a first side opposite a second side, and a first surface opposite a second surface. A width of the sensor tail measured from the first side to the opposing second side of the tail in a direction transverse to the longitudinal axis of the tail is in a range of 150 μm to 250 μm. The analyte sensor further includes a flag, also referred to as a body or body portion, having an electrical contact electrically connected to the working electrode and a neck connecting the sensor tail and the flag.

In any of the various embodiments described herein, the width of the sensor tail may be in a range of 170 μm to 230 μm.

In any of the various embodiments described herein, the width of the sensor tail may be approximately the same as a width of the active area.

In any of the various embodiments described herein, a ratio of the width of the sensor tail to a width of the active area may be in a range of 1:1 to 1.7:1.

In any of the various embodiments, the substrate and the working electrode may each have a first side opposite a second side and a first surface opposite a second surface extending across at least a part of the width of the sensor tail, and the active area may be disposed on a surface of the working electrode.

In any of the various embodiments described herein, a distance from a first side and/or a second side of the substrate to an edge of the active area, and/or a distance from a first side and/or a second side of the sensor tail to an edge of the active area, may be in a range of 10 μm to 90 μm.

In any of the various embodiments described herein, the analyte sensor further includes a counter electrode or a reference electrode.

In any of the various embodiments described herein, the sensor control device further includes an adhesive patch attached to a bottom of the housing and configured to secure the sensor control device to a skin surface of a user.

In any of the various embodiments described herein, the sensor control device further includes a plug assembly coupled to the electronics housing and including a sensor module and a sharp module, wherein the sensor module includes the analyte sensor.

Some embodiments described herein relate to a method for manufacturing an analyte sensor for monitoring analyte levels. The method includes forming a substrate, depositing a conductive layer on the substrate to form a working electrode, depositing a reagent composition on the working electrode to form an active area, and cutting the sensor tail along a longitudinal axis of the sensor tail to reduce the width of the sensor tail to a range of 150 μm to 250 μm, wherein the width is measured in a direction transverse to the longitudinal axis of the sensor tail from a first side to an opposing second side of the sensor tail.

In any of the various embodiments described herein, cutting the sensor tail includes cutting along the first side of the sensor tail in a longitudinal direction. In some embodiments, cutting the sensor tail further includes cutting along the second side of the sensor tail opposite the first side in the longitudinal direction.

In any of the various embodiments described herein, cutting the sensor tail is performed by laser cutting.

In any of the various embodiments described herein, the method for manufacturing the analyte sensor may further include applying a membrane to overcoat at least the active area of the sensor tail.

In any of the various embodiments described herein, the sensor tail is cut such that a ratio of the width of the sensor tail to a width of the active area is in a range of 1:1 to 1.7:1.

In the embodiments described herein, references to the width of the sensor tail can also be taken to refer to the width of the substrate or each electrode. For example, where more than one electrode is provided on the substrate. This may be the case where the analyte sensor comprises multiple electrodes, for monitoring multiple analytes, as described above.

### BRIEF DESCRIPTION OF THE DRAWINGS

The accompanying drawings, which are incorporated herein and form a part of the specification, illustrate the present disclosure and, together with the description, further serve to explain the principles thereof and to enable a person skilled in the pertinent art to make and use the same.

FIG. 1 shows a plan view of an analyte sensor having a planar substrate layer for embodiments of the invention.

FIG. 2 shows a longitudinal cross sectional view of a portion of an analyte sensor according to an embodiment.

FIG. 3 shows a perspective view of the analyte sensor of FIG. 1 arranged in a tubular configuration.

FIG. 4 shows a transverse cross sectional view of the analyte sensor of FIG. 3.

FIG. 5 shows a perspective view of the analyte sensor of FIG. 3 with extensions positioned for connection to sensor electronics according to an embodiment.

FIG. 6 shows a plan view of an analyte sensor having a planar substrate layer for embodiments of the invention.

FIG. 7 shows a perspective view of the analyte sensor of FIG. 6 arranged in a tubular configuration.

FIG. 8 shows a perspective view of the analyte sensor of FIG. 7 with the extension positioned for connection to sensor electronics according to an embodiment.

FIG. 9 shows an exemplary method of forming an analyte sensor according to an embodiment.

FIG. 10 shows an analyte sensor as part of an analyte monitoring assembly according to an embodiment.

FIGS. 11A and 11B show block diagrams depicting exemplary embodiments of analyte sensors and sensor electronics according to embodiments.

FIG. 12 shows an analyte monitoring system according to an embodiment.

FIG. 13 shows a block diagram depicting a receiver device.

FIG. 14 shows a perspective view of an analyte monitoring assembly according to an embodiment.

FIG. 15 shows an exploded view of an analyte monitoring assembly according to an embodiment.

FIG. 16 shows a perspective view of the analyte monitoring assembly of FIG. 15 in an assembled configuration.

FIGS. 17A and 17B show schematic cross sectional views of a sensor tail of an analyte sensor according to some embodiments.

FIG. 18 shows an exploded view of a portion of an analyte monitoring assembly according to an embodiment.

FIG. 19 shows a perspective view of the portion of the analyte monitoring assembly of FIG. 18 in an assembled configuration.

FIG. 20 shows an exploded view of a portion of an analyte monitoring assembly according to an embodiment.

FIG. 21 shows a perspective view of the portion of the analyte monitoring assembly of FIG. 20 in an assembled configuration.

FIG. 22 shows an exploded view of a portion of an analyte monitoring assembly according to an embodiment.

FIG. 23 shows a perspective view of the portion analyte monitoring assembly of FIG. 22 in an assembled configuration.

FIG. 24 shows a bottom view of an analyte monitoring assembly according to an embodiment.

FIG. 25 shows a bottom view of an analyte monitoring assembly according to an embodiment.

FIG. 26 shows a bottom view of an analyte monitoring assembly according to an embodiment.

FIG. 27 shows an exploded view of an analyte sensor and insertion device according to an embodiment.

FIG. 28 shows a perspective view of an analyte sensor and insertion device of FIG. 27 as positioned for insertion of the analyte sensor.

FIG. 29 shows a series of steps for manufacturing an insertion device according to an embodiment.

FIG. 30 shows an exploded view of a plug assembly for an analyte monitoring assembly according to an embodiment.

FIG. 31 shows a perspective view of the assembled plug assembly of FIG. 30.

FIG. 32 shows an exploded view of an analyte monitoring assembly including the plug assembly of FIG. 31.

FIG. 33 shows a side view of a fully assembled analyte monitoring assembly of FIG. 32.

FIG. 34A shows a perspective view of a sensor control device according to some embodiments.

FIG. 34B shows a side view of the sensor control device of FIG. 34A.

FIG. 35A shows a perspective view of a plug assembly for a sensor control device according to some embodiments.

FIG. 35B is an exploded view of the plug assembly of FIG. 36A.

FIGS. 36A and 36B are exploded and bottom isometric views of electronics housing of sensor control device according to some embodiments.

FIG. 37 shows a plan view of an analyte sensor having a reduced profile sensor tail according to some embodiments.

FIGS. 38A and 38B show a sensor tail and a reduced profile sensor tail, respectively, for a sensor control device according to some embodiments.

FIG. 39 shows a schematic diagram of a reduced profile sensor tail for a sensor control device according to some embodiments.

FIG. 40 shows a cross sectional view of a reduced profile sensor tail for a sensor control device according to some embodiments.

FIG. 41 is a flowchart of an exemplary method to manufacture a reduced profile sensor tail for a sensor control device according to some embodiments.

FIG. 42 shows a perspective view of an analyte sensor having a tubular configuration and reduced width electrodes according to an embodiment.

### DETAILED DESCRIPTION

Reference will now be made in detail to representative embodiments illustrated in the accompanying drawings. It should be understood that the following descriptions are not intended to limit the embodiments to one preferred embodiment. To the contrary, it is intended to cover alternatives, modifications, and equivalents as can be included within the spirit and scope of the described embodiments as defined by the claims.

As used herein and in the appended claims, the singular forms “a,” “an,” and “the” include plural referents unless the context clearly dictates otherwise.

In the following description, numerous specific details are set forth in order to provide a thorough understanding of the embodiments of the present disclosure. However, it will be apparent to those skilled in the art that the embodiments, including structures, systems, and methods, may be practiced without these specific details. The description and representation herein comport with standards used by those experienced or skilled in the art to most effectively convey the substance of their work to others skilled in the art. In some instances, well-known methods, procedures, and components have not been described in detail to avoid unnecessarily obscuring aspects of the disclosure.

References in the specification to “some embodiments” indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to apply such feature, structure, or characteristic in connection with other embodiments whether or not explicitly described.

The following examples are illustrative, but not limiting, of the present disclosure. Other suitable modifications and adaptations of the variety of conditions and parameters normally encountered in the field, and which would be apparent to those skilled in the art, are within the spirit and scope of the disclosure.

As used herein, the term “approximately” means ±10% of the target value unless indicated otherwise.

The term “analyte” as used herein, may refer to, for example, glucose, ketones (including beta-hydroxybutyrate, acetone, or acetoacetate), lactate, oxygen, hemoglobin A1C, albumin, alcohols (e.g., ethanol), alkaline phosphatase, alanine transaminase, aspartate aminotransferase, bilirubin, blood urea nitrogen, calcium, carbon dioxide, chloride, creatinine, hematocrit, lactate, magnesium, oxygen, pH, phosphorus, potassium, sodium, total protein, and uric acid, among others.

The term “biological fluid,” as used herein, refers to any bodily fluid or bodily fluid derivative in which the analyte can be measured. Non-limiting examples of a biological fluid include dermal fluid, interstitial fluid, plasma, blood, lymph, synovial fluid, cerebrospinal fluid, saliva, bronchoalveolar lavage, amniotic fluid, sweat, tears or the like. In certain embodiments, the biological fluid is dermal fluid or interstitial fluid.

Monitoring multiple analytes can be beneficial to patients. Monitoring multiple analytes may allow for investigation into the impacts of a particular treatment or the effects of a medication or behavior modification. Monitoring multiple analytes may also help to identify illness or the severity of a disease state. Continuous data for multiple analytes may further illuminate other patterns or trends.

However, wearing multiple separate analyte monitoring devices that each monitor a single analyte may be inconvenient for the user. Each analyte monitoring device may occupy space on the wearer's body. Each analyte monitoring device may require separate insertion, subjecting the patient to multiple needle insertions. Each analyte monitor may require a separate procedure for pairing the analyte monitoring device to a receiver device, such as a smartphone of the user. The user may have to track the wear-time or life of each analyte monitoring device separately and replace the analyte monitoring devices at different intervals (e.g., a first analyte monitoring device may have a wear time of 14 days while another may have a wear time of 10 days). These various issues can result in a cumbersome and inconvenient experience for the user which may deter monitoring multiple analytes.

Some analyte monitoring devices may include a single sensor tail inserted under the skin for detecting multiple analytes in a bodily fluid. However, the small size of the sensor tail makes it difficult to include two or more analyte sensing areas. Increasing the size of the sensor tail to accommodate multiple sensing areas may be undesirable, as the larger sensor tail may make insertion more painful. Thus, there is a need for an improved analyte sensor and analyte monitoring device for monitoring multiple analytes in a bodily fluid.

Some embodiments described herein relate to an in vivo analyte sensor configured to be inserted at least partially under a skin surface of a user and in contact with a bodily fluid for measuring analyte levels in the bodily fluid. The in vivo analyte sensor has a substrate layer that is arranged in a tubular configuration, with multiple electrodes for detecting multiple analytes in the bodily fluid. One or more of the electrodes include sensing areas, also referred to as active areas or sensing elements, for detecting analytes. Sensing areas include reagents disposed on the electrodes, such as an analyte-responsive enzyme, to aid in detection of analytes. The substrate layer, for example a planar substrate layer, may be arranged in a tubular configuration, such as by winding the substrate layer around a longitudinal axis. The tubular configuration of the substrate layer may increase the surface area on which sensing areas may be placed. Each electrode may be electrically connected to an electrical contact configured to be connected to sensor electronics of an analyte monitoring assembly for communicating signals indicative of analyte levels as detected by the electrodes.

In this way, a single analyte sensor can be used to monitor multiple analytes. As a result, multiple analytes can be monitored by insertion of a single analyte sensor into the user, alleviating the discomfort of multiple sensor insertions. Further, the use of a single analyte sensor and analyte monitoring assembly allows for a single pairing operation to be performed to pair the analyte monitoring assembly to a receiver device. The user may thereafter receive and view analyte data for the multiple monitored analytes on the receiver device.

As the in vivo analyte sensor detects multiple analytes, the user may use the same device whether detecting a first analyte or a second analyte, or both. For example, if the user is monitoring a first analyte and wants to begin monitoring a second analyte, the analyte monitoring device may already be configured to monitor that second analyte such that the user may not need to install a new analyte monitoring device that is particular to the second analyte. Additionally, the user can continue to use previously purchased analyte monitoring devices and need not purchase new analyte sensors specific to monitoring the second analyte. In other examples, more than two analytes may be detected.

Analyte monitoring devices can be used to continuously or periodically monitor an analyte level in a bodily fluid of a user, such as blood or interstitial fluid, among others. Analyte sensors typically include a single sensor tail that is inserted under the skin of the user to measure analyte levels in the bodily fluid. If the sensor tail fails to perform accurately or reliably, the analyte sensor must be discarded and a new analyte monitoring device installed. This can be inconvenient for the user to replace the sensor, and further the insertion of the sensor may cause discomfort to the user, rendering the need for frequent sensor replacements undesirable.

Some users may wish to monitor the level of multiple analytes in a bodily fluid. If each analyte monitoring device is configured to monitor one analyte, then the user must wear two or more analyte monitoring devices to monitor each analyte of interest. This can be inconvenient, as the analyte monitoring devices consume space on the body of the user, and it may be undesirable to wear multiple different analyte monitoring devices. Further, the user will experience multiple sensor insertions which may also be uncomfortable for the user.

In order to monitor multiple analytes, a single analyte monitoring device may be provided that includes a sensor tail with two or more active areas for monitoring two or more analytes. However, the sensing structures and sensing chemistry for monitoring analytes requires a certain minimum amount of space on the sensor tail. As such, the sensor tail is constructed to accommodate the multiple sensing structures and chemistries. As a result, it can be difficult or impractical to incorporate multiple active areas on a single sensor tail, and doing so limits the ability to reduce the size of the sensor tail. Manufacturing may be made more complex by the need to fabricate a sensor tail with multiple active areas which may require application of multiple reagents and coatings on specific portions of the sensor tail.

To address these problems, the present application relates to an analyte monitoring assembly including an analyte sensor with a body having multiple sensor tails for detecting analytes. A size of each sensor tail can be minimized by including a single electrode on each sensor tail. A sensor tail may include only a single electrode that is a working electrode with an analyte-responsive enzyme for detecting a single analyte. In some embodiments, additional sensor tails of the analyte sensor may include a counter electrode or reference electrode. The reduced size of the sensor tail facilitates insertion and reduces the pain of insertion of the analyte sensors.

Further, multiple analyte sensor tails may be used to detect the same analyte, and data from a first sensor tail may be compared with data collected by a second sensor tail to help determine an accurate analyte value, or to detect an error in operation of one of the senor tails. In the event one sensor tail is determined to be functioning improperly, the remaining sensor tails may still be used, avoiding the need to replace the analyte monitoring assembly.

The analyte sensor having multiple sensor tails may facilitate manufacturing and assembly of the analyte monitoring assembly. Rather than separately manufacturing and assembling a plurality of separate analyte sensors each having one sensor tail, one analyte sensor includes all sensor tails such that analyte sensor can be easily assembled with housing and sensor electronics of analyte monitoring assembly. Further, sensor tails can be serially processed, such as by applying reagents, coatings, or membranes to the individual sensor tails of the analyte sensor during manufacturing.

The detection and/or monitoring of bodily fluid analytes in certain individuals may be vitally important to the individual's health. For example, the monitoring of glucose is particularly important to individuals with diabetes. Patients with diabetes generally monitor glucose levels to determine if their glucose levels are being maintained within a clinically safe range, and may also use this information to determine if and/or when insulin is needed to reduce glucose levels in their bodies or when additional glucose is needed to raise the level of glucose in their bodies.

Analyte monitoring may also be used for health and wellness. Users may track analyte levels to help determine the impact of foods and exercise on glucose levels to help optimize diet and exercise plans for achieving various fitness goals. Further, users may track ketone levels to determine the status of a diet and determine if fat burning is occurring.

Analyte monitoring in an individual can take place periodically or continuously over a period of time. Periodic analyte monitoring can take place by withdrawing a sample of bodily fluid, such as blood or urine, at set time intervals and analyzing the sample of the bodily fluid ex vivo. Periodic, ex vivo analyte monitoring can be sufficient to determine the physiological condition of many individuals. However, ex vivo analyte monitoring can be inconvenient or painful in some instances. Moreover, there is no way to recover lost data if an analyte measurement is not obtained at an appropriate time.

Continuous analyte monitoring can be conducted using one or more in vivo analyte sensors positioned at least partially below the skin surface of an individual, such as dermally, subcutaneously or intravenously, so that analyses can be conducted in vivo. In vivo analyte sensors can collect analyte data on-demand, at a set schedule, or continuously, depending on an individual's particular health needs and/or previously measured analyte levels. Analyte monitoring with an in vivo analyte sensor can be a more desirable approach for individuals having severe analyte dysregulation and/or rapidly fluctuating analyte levels, although it can also be beneficial for other individuals as well.

Analyte monitoring systems, for example, have been developed to facilitate continuous monitoring of bodily fluid analytes. Analyte monitoring systems typically include a biological sensor and a sensor applicator configured to place the biological sensor into contact with a bodily fluid. More specifically, during delivery of the sensor to the skin of a user, at least a portion of the sensor (e.g. a sensor tail) is inserted and positioned below the skin surface, e.g., in the subcutaneous or dermal tissue.

The sensor tail inserted and positioned below the skin surface includes sensing chemistry for detecting the presence of an analyte in the bodily fluid. The sensing chemistry may include one or more enzymes, such as an analyte responsive enzyme as discussed in further detail herein. Sensing chemistry may include additional reagents and components to facilitate detection of the analyte of interest. In some embodiments, one or more membranes may be placed over and may at least partially cover the sensing chemistry.

However, the sensor tail inserted and positioned below the skin surface can cause a foreign body response, which may interfere with sensor accuracy over the time the analyte sensor is worn. Foreign body response is an inflammatory and fibrotic process, during which fibroblasts gradually envelop the foreign material and form a physical barrier to isolate it from the rest of the body. Thus, the response disrupts the interface between the sensor and its target tissue as time increases. For example, an analyte sensor for monitoring glucose can be implanted under skin surface for a predetermined period of time, after which the glucose consumption by macrophages surrounding the sensor tail may result in lower glucose readings.

This issue can be addressed by incorporating anti-inflammatory drugs into the sensor. However, the use of anti-inflammatory drugs may increase the manufacturing burden in producing the sensor and may require the sensor to be subjected to further review and evaluation as part of a complex regulatory approval process.

Another solution to reduce the foreign body response is to reduce the profile of the sensor tail, so that the sensor accuracy can be maintained for a longer time in vivo. A reduced profile sensor tail has less manufacturing burden, as it requires no new materials, and it also lowers the risk of biological safety and with less regulatory complexity. Reducing the profile of the sensor tail may further facilitate insertion of the sensor tail under the skin of the user. Generally, smaller diameter or width sensors or needles are less painful to be inserted relative to larger diameter or width sensors or needles. Pain of insertion may cause some users to avoid using in vivo sensors or to discontinue use of in vivo sensors. Thus, reducing the sensor profile further promotes user comfort and compliance with analyte monitoring programs.

Some embodiments described herein relate to an analyte sensor having a sensor tail for insertion under the skin and having sensing chemistry disposed on the sensor tail for detecting an analyte in a bodily fluid. Analyte sensor may further include a flag, also referred to as a body or body portion, with electrical contacts and a neck connecting the flag and sensor tail. The flag may be configured to be arranged outside of a body of a user for connection to sensor electronics of a sensor control device. The sensing chemistry may include one or more spots of a reagent composition. A width of the sensor tail in a direction transverse to a longitudinal axis of the sensor tail may be the same as or slightly greater than a width of a diameter of the spot of the reagent composition. In some embodiments, the sensor tail has a width of 150 μm to 250 μm. In some embodiments, a ratio of the width of the sensor tail to a width of the active area is in a range of 1:1 to 1.7:1. In some embodiments, a distance from an edge of the spot of the reagent to a side of the sensor tail is in a range of 10 μm to 90 μm.

Analyte sensors as described herein may be part of a sensor control device. The sensor control device may include an electronics housing containing sensor electronics. Sensor electronics may be in communication with the analyte sensor. The sensor electronics may include a processor, memory coupled to the processor and storing instructions to be executed by the processor. The sensor electronics may include a power source. The sensor electronics may include communication circuitry, such as wireless communication circuitry, such as a Bluetooth antenna for transmitting data, such as monitored analyte data, to a remote device, such as a reader device. Reader device may display at least a portion of the analyte data, or analyte metrics derived from the analyte data to a user, such as to a patient, patient's guardian or caregiver, or to a healthcare professional.

An analyte sensor may include a substrate layer having a plurality of electrodes disposed thereon. The substrate layer may be arranged in a tubular configuration. A conductive material may be disposed on a surface of the substrate layer to form the plurality of electrodes. The plurality of electrodes may be separated from one another by one or more boundaries. The boundaries may include an area in which conductive material is not present. The electrodes may include one or more of each of a working electrode, a counter electrode, and a reference electrode. Analyte sensor may include multiple working electrodes for detecting the same analyte or different analytes. An active area may be disposed on one or more electrodes for facilitating detection of an analyte of interest. The analyte sensor may include a membrane covering one or more of the electrodes and/or active areas. Each electrode may be connected to an electrical contact for communication with sensor electronics of an analyte monitoring assembly. Analyte sensor may include a first portion to be arranged below the skin that includes the tubular structure, and a second portion arranged above the skin that includes the electrical contacts for connection to sensor electronics.

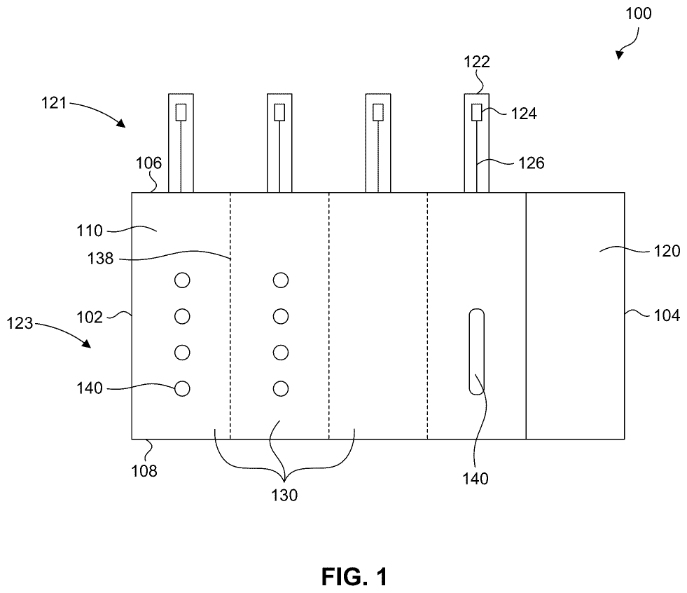

A planar in vivo analyte sensor according to an embodiment is shown for example in FIG. 1. Analyte sensor 100 may include a substrate layer 110, a plurality of electrodes 130 disposed on the substrate layer 110, and reagents 140 for detecting an analyte are disposed on one or more of electrodes 130. Each electrode 130 may be in electrical communication with an electrical contact 124 for communicating signals collected by electrodes to sensor electronics of an analyte monitoring assembly, such as by electrical traces 126. Analyte sensor 100 may include a first portion 121, also known as a body or body portion, configured to be arranged above a skin surface of a user and in contact with sensor electronics of an analyte monitoring assembly, and a second portion 123, also known as a tail or tail portion, configured to be inserted under the skin surface and into contact with a bodily fluid, such as blood or interstitial fluid for sensing analytes in the bodily fluid. Electrodes 130 and reagents 140 are arranged on the second portion 123 of analyte sensor 100 that is configured to be arranged under the skin surface.

Planar substrate layer 110 may have a generally rectangular shape with a first side 102 opposite a second side 104 in a transverse direction of substrate layer 110 and a first end 106 opposite a second end 108 in a longitudinal direction of substrate layer 110, wherein the longitudinal direction is perpendicular to the transverse direction. Substrate layer 110 may be formed as a thin sheet or layer of material, and may be substantially planar (prior to being arranged in a tubular shape as described herein). Substrate layer 110 of analyte sensor 100 may be formed from a non-conductive material. In some embodiments, substrate layer 110 may include polyethylene terephthalate (PET), polyester, polyimide, or a combination thereof, among other materials. Substrate layer 110 may be flexible so as to be capable of being arranged in a desired configuration, such as a tubular shape. Substrate layer 110 has sufficient rigidity to maintain its tubular shape, such as when inserted into tissue of the user.

Electrodes 130 may be arranged on a surface of substrate layer 110. Electrodes 130 may include a layer of conductive material disposed on a surface of substrate layer 110. In some embodiments, electrodes 130 may include carbon disposed on substrate layer 110. Electrodes 130 may be electrically isolated from one another. In some embodiments, conductive material may be disposed on substrate layer 110 and boundaries 138 are formed between electrodes 130 by removing a portion of the conductive material, such as by laser ablation or etching. In some embodiments, conductive material of electrodes 130 is deposited on substrate layer 110 individually to form each electrode 130 with electrodes 130 being spaced from one another to form boundaries 138 such that a subsequent laser ablation or etching step is not required.

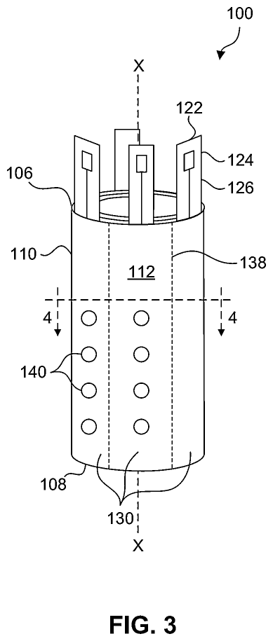

In some embodiments, boundaries 138 may be substantially linear. Boundaries 138 may extend in a direction of a longitudinal axis of substrate layer 110 (e.g., FIG. 3) from first end 106 to second end 108. Each electrode 130 may extend between first end 106 and second end 108 of substrate layer 110, and may extend fully from first end 106 to second end 108. Multiple electrodes 130 may be arranged side-by-side between opposing sides 102, 104 of substrate layer 110. In some embodiments, analyte sensor 100 may include 2 to 8 electrodes, 3 to 7 electrodes, or 4 to 6 electrodes.

In some embodiments, electrodes 130 may cover substantially an entire surface of substrate layer 110 from first end 106 to second end 108 and from first side 102 to opposing second side 104. In some embodiments, electrodes 130 are disposed on a region of substrate layer 110 and a second region is not used to form electrodes 130. A core 120 may include the region of substrate layer 110 where no electrodes are formed. Core 120 may be arranged at second side 104 of substrate layer 110. However, core 120 may alternately be arranged at first side 102 of substrate layer 110. In some embodiments, core 120 may have a greater thickness than a remainder of substrate layer 110.

Reagents 140 may be disposed on one or more of electrodes 130. Reagents 140 may be arranged in one or more locations on each electrode. Reagent 140 may be deposited on an electrode in one or more spots. Spots may be arranged in a line and/or in an array, and may be spaced from one another each at an interval, which may be fixed intervals between multiples spots. In some embodiments, reagents 140 may be deposited on electrode 130 as a continuous line. In some embodiments, reagent 140 may include an analyte-responsive enzyme, as discussed in further detail herein.

In some embodiments, analyte sensor 100 may include multiple working electrodes. Each electrode 130 may be configured to detect a different analyte. A first electrode 130 may include a first reagent 140 for detecting a first analyte, and a second electrode 130 may include a second reagent 140 for detecting a second analyte. For example, a first electrode may be used to detect glucose and a second electrode may be used to detect lactate. Analyte sensor 100 may include additional working electrodes having reagents for detecting additional analytes, e.g., a third analyte, a fourth analyte, a fifth analyte, etc. In some embodiments, two or more electrodes may be configured to detect the same analyte. Multiple electrodes detecting the same analyte may provide redundancy, and the redundant electrodes can be used for error detection or for improving the accuracy of the determined analyte level.

In some embodiments, one or more electrodes may serve as a counter electrode or a reference electrode. Reference electrode may include a reference material layer, such as silver (Ag) or silver chloride (AgCl) disposed on the conductive material. In some embodiments, a dielectric layer may be disposed on the reference material layer. At least a portion of reference material layer may be exposed to the bodily fluid via an opening in dielectric layer.

Various combinations of electrodes are possible. For example, in some embodiments, analyte sensor 100 may include a first working electrode and a second working electrode. In some embodiments, analyte sensor 100 may include a first working electrode, a second working electrode, and either a counter electrode or a reference electrode. In some embodiments, analyte sensor 100 may include a first working electrode, a second working electrode, a counter electrode, and a reference electrode. Additional working electrodes may be added depending on the number of analytes desired to be monitored.

Each electrode 130 of analyte sensor 100 may be electrically connected to an electrical contact 124. Electrical contact 124 may be configured to provide electrical connection to sensor electronics of an analyte monitoring assembly outside of a body of a user. Electrodes 130 may be connected to electrical contacts 124 by electrical traces 126. In some embodiments, substrate layer 110 may include a plurality of extensions 122 extending from substrate layer 110. In FIG. 1, extensions 122 extend from first end 106 of substrate layer 110. In some embodiments, extensions 122 may include elongated strips. Extensions 122 may have a width that is narrower than a width of the corresponding electrode. Extensions 122 may be spaced from one another. Each extension 122 may include an electrical contact 124 for electrical connection to sensor electronics of an analyte monitoring assembly outside of a body of a user. Each electrical contact 124 may be in electrical communication with a corresponding electrode 130 via an electrical trace 126.

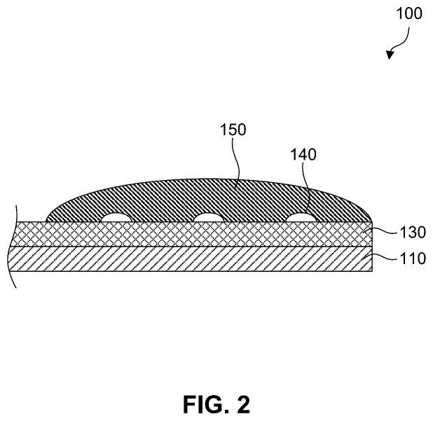

As shown in FIG. 2, analyte sensor 100 may include a substrate layer 110, conductive material disposed on a surface of substrate layer 110 to form an electrode 130. One or more reagents 140 are disposed on electrode 130, such as an analyte-responsive enzyme. In FIG. 2, the reagent 140 is shown as deposited in multiple spots or drops. However, in other embodiments, the reagent 140 may be deposited as a continuous line or as a single spot. The electrodes 130 and reagents 140 may be encapsulated by one or more membranes 150. Membrane 150 may be a polymeric membrane. Membrane 150 may include a diffusion or mass-transport limiting membrane. Membrane 150 may improve biocompatibility and/or may serve to reduce or inhibit interferents.

In some embodiments, analyte sensor 100 may include an analyte-responsive enzyme to provide a sensing element, also referred to herein as an active area. Some analytes, such as oxygen, can be directly electrooxidized or electroreduced at least on a working electrode of analyte sensor 100. Other analytes, such as glucose and lactate, require the presence of at least one electron transfer agent and/or at least one catalyst to facilitate the electrooxidation or electroreduction of the analyte. Catalysts may also be used for those analytes, such as oxygen, that can be directly electrooxidized or electroreduced on the working electrode. For these analytes, each working electrode includes a sensing element proximate to or on a surface of a working electrode. In many embodiments, a sensing element is formed near or on only a small portion of at least a working electrode.

Each sensing element includes one or more components constructed to facilitate the electrochemical oxidation or reduction of the analyte. The sensing element may include, for example, a catalyst to catalyze a reaction of the analyte and produce a response at the working electrode, an electron transfer agent to transfer electrons between the analyte and the working electrode (or other component), or both.

A variety of different sensing element configurations may be used. In certain embodiments, the sensing elements are deposited on the conductive material of a working electrode. The sensing elements may extend beyond the conductive material of the working electrode. In some cases, the sensing elements may also extend over other electrodes, e.g., over the counter electrode and/or reference electrode (or counter/reference where provided). In other embodiments, the sensing elements are contained on the working electrode, such that the sensing elements do not extend beyond the conductive material of the working electrode. In some embodiments a working electrode is configured to include a plurality of spatially distinct sensing elements. Additional information related to the use of spatially distinct sensing elements can be found in U.S. Pat. No. 10,327,677, which was filed on Dec. 8, 2011, and which is incorporated by reference herein in its entirety and for all purposes.

The terms “working electrode”, “counter electrode”, “reference electrode” and “counter/reference electrode” are used herein to refer to conductive sensor components, including, e.g., conductive traces, which are configured to function as a working electrode, counter electrode, reference electrode or a counter/reference electrode respectively. For example, a working electrode includes that portion of a conductive material, e.g., a conductive trace, which functions as a working electrode as described herein, e.g., that portion of a conductive material which is exposed to an environment containing the analyte or analytes to be measured, and which, in some cases, has been modified with one or more active areas as described herein. Similarly, a reference electrode includes that portion of a conductive material, e.g., conductive trace, which function as a reference electrode as described herein, e.g., that portion of a conductive material which is exposed to an environment containing the analyte or analytes to be measured, and which, in some cases, includes a secondary conductive layer, e.g., a Ag/AgCl layer. A counter electrode includes that portion of a conductive material, e.g., conductive trace which is configured to function as a counter electrode as described herein, e.g., that portion of a conductive trace which is exposed to an environment containing the analyte or analytes to be measured. As noted above, in some embodiments, a portion of a conductive material, e.g., conductive trace, may function as either or both of a counter electrode and a reference electrode. In addition, “working electrodes”, “counter electrodes”, “reference electrodes” and “counter/reference electrodes” may include portions, e.g., conductive traces, electrical contacts, or areas or portions thereof, which do not include active areas but which are used to electrically connect the electrodes to other electrical components.

Sensing elements that are in direct contact with the working electrode, e.g., the working electrode trace, may contain an electron transfer agent to transfer electrons directly or indirectly between the analyte and the working electrode, and/or a catalyst to facilitate a reaction of the analyte. For example, a glucose, lactate, or oxygen electrode may be formed having sensing elements which contain a catalyst, including glucose oxidase, glucose dehydrogenase, lactate oxidase, or laccase, respectively, and an electron transfer agent that facilitates the electrooxidation of the glucose, lactate, or oxygen, respectively.

In some embodiments, the sensing elements are not deposited directly on the working electrode, e.g., the working electrode trace. Instead, the sensing elements may be spaced apart from the working electrode or working electrode trace, and separated from the working electrode or working electrode trace, e.g., by a separation layer. A separation layer may include one or more membranes or films or a physical distance. In addition to separating the working electrode or working electrode trace from the sensing elements, the separation layer may also act as one or more of a mass transport limiting layer, an interferent eliminating layer, or a biocompatibility layer.

In embodiments that include more than one working electrode, one or more of the working electrodes may not have corresponding sensing elements, or may have sensing elements that do not contain one or more components (e.g., an electron transfer agent and/or catalyst) needed to electrolyze the analyte. Thus, the signal at this working electrode may correspond to background signal which may be removed from the analyte signal obtained from one or more other working electrodes that are associated with fully-functional sensing elements by, for example, subtracting the signal.

In some embodiments, the sensing elements include one or more electron transfer agents. Electron transfer agents that may be employed are electroreducible and electrooxidizable ions or molecules having redox potentials that are a few hundred millivolts above or below the redox potential of the standard calomel electrode (SCE). The electron transfer agent may be organic, organometallic, or inorganic. Examples of organic redox species are quinones and species that in their oxidized state have quinoid structures, such as Nile blue and indophenol. Examples of organometallic redox species are metallocenes including ferrocene. Examples of inorganic redox species are hexacyanoferrate (III), ruthenium hexamine, etc. Additional examples include those described in U.S. Pat. Nos. 6,736,957, 7,501,053, and 7,754,093, the disclosures of each of which are incorporated herein by reference in their entireties.

In certain embodiments, electron transfer agents have structures or charges which prevent or substantially reduce the diffusional loss of the electron transfer agent during the period of time that the sample is being analyzed. For example, electron transfer agents include but are not limited to a redox species, e.g., bound to a polymer which can in turn be disposed on or near the working electrode. The bond between the redox species and the polymer may be covalent, coordinative, or ionic. Although any organic, organometallic or inorganic redox species may be bound to a polymer and used as an electron transfer agent, in certain embodiments the redox species is a transition metal compound or complex, e.g., osmium, ruthenium, iron, and cobalt compounds or complexes. It will be recognized that many redox species described for use with a polymeric component may also be used, without a polymeric component.

Embodiments of polymeric electron transfer agents may contain a redox species covalently bound in a polymeric composition. An example of this type of mediator is poly(vinylferrocene). Another type of electron transfer agent contains an ionically-bound redox species. This type of mediator may include a charged polymer coupled to an oppositely charged redox species. Examples of this type of mediator include a negatively charged polymer coupled to a positively charged redox species such as an osmium or ruthenium polypyridyl cation.

Another example of an ionically-bound mediator is a positively charged polymer including quaternized poly (4-vinyl pyridine) or poly(1-vinyl imidazole) coupled to a negatively charged redox species such as ferricyanide or ferrocyanide. In other embodiments, electron transfer agents include a redox species coordinatively bound to a polymer. For example, the mediator may be formed by coordination of an osmium or cobalt 2,2′-bipyridyl complex to poly(1-vinyl imidazole) or poly(4-vinyl pyridine).

Suitable electron transfer agents are osmium transition metal complexes with one or more ligands, each ligand having a nitrogen-containing heterocycle such as 2,2′-bipyridine, 1,10-phenanthroline, 1-methyl, 2-pyridyl biimidazole, or derivatives thereof. The electron transfer agents may also have one or more ligands covalently bound in a polymer, each ligand having at least one nitrogen-containing heterocycle, such as pyridine, imidazole, or derivatives thereof. One example of an electron transfer agent includes (a) a polymer or copolymer having pyridine or imidazole functional groups and (b) osmium cations complexed with two ligands, each ligand containing 2,2′-bipyridine, 1,10-phenanthroline, or derivatives thereof, the two ligands not necessarily being the same. Some derivatives of 2,2′-bipyridine for complexation with the osmium cation include but are not limited to 4,4′-dimethyl-2,2′-bipyridine and mono-, di-, and polyalkoxy-2,2′-bipyridines, including 4,4′-dimethoxy-2,2′-bipyridine. Derivatives of 1,10-phenanthroline for complexation with the osmium cation include but are not limited to 4,7-dimethyl-1,10-phenanthroline and mono, di-, and polyalkoxy-1,10-phenanthrolines, such as 4,7-dimethoxy-1,10-phenanthroline. Polymers for complexation with the osmium cation include but are not limited to polymers and copolymers of poly(1-vinyl imidazole) (referred to as “PVI”) and poly(4-vinyl pyridine) (referred to as “PVP”). Suitable copolymer substituents of poly(1-vinyl imidazole) include acrylonitrile, acrylamide, and substituted or quaternized N-vinyl imidazole, e.g., electron transfer agents with osmium complexed to a polymer or copolymer of poly(1-vinyl imidazole).

In some embodiments, electron transfer agents may have a redox potential ranging from about −200 mV to about +200 mV versus the standard calomel electrode (SCE). The sensing elements may also include a catalyst which is capable of catalyzing a reaction of the analyte. The catalyst may also, in some embodiments, act as an electron transfer agent. One example of a suitable catalyst is an enzyme which catalyzes a reaction of the analyte. For example, a catalyst, including a glucose oxidase, glucose dehydrogenase (e.g., pyrroloquinoline quinone (PQQ), dependent glucose dehydrogenase, flavine adenine dinucleotide (FAD) dependent glucose dehydrogenase, or nicotinamide adenine dinucleotide (NAD) dependent glucose dehydrogenase), may be used when the analyte of interest is glucose. A lactate oxidase or lactate dehydrogenase may be used when the analyte of interest is lactate. Laccase may be used when the analyte of interest is oxygen or when oxygen is generated or consumed in response to a reaction of the analyte.

In certain embodiments, a catalyst may be attached to a polymer, cross linking the catalyst with another electron transfer agent, which, as described above, may be polymeric. A second catalyst may also be used in certain embodiments. This second catalyst may be used to catalyze a reaction of a product compound resulting from the catalyzed reaction of the analyte. The second catalyst may operate with an electron transfer agent to electrolyze the product compound to generate a signal at the working electrode. Alternatively, a second catalyst may be provided in an interferent-eliminating layer to catalyze reactions that remove interferents.

In certain embodiments, the sensor works at a low oxidizing potential, e.g., a potential of about +40 mV vs. Ag/AgCl. These sensing elements use, for example, an osmium (Os)-based mediator constructed for low potential operation. Accordingly, in certain embodiments the sensing elements are redox active components that include: (1) osmium-based mediator molecules that include (bidente) ligands, and (2) glucose oxidase enzyme molecules. These two constituents are combined together in the sensing elements of the sensor.

A mass transport limiting layer, e.g., an analyte flux modulating layer, may be included with the analyte sensor to act as a diffusion-limiting barrier to reduce the rate of mass transport of the analyte, for example, glucose or lactate, into the region around the working electrode(s). The mass transport limiting layers are useful in limiting the flux of an analyte to a working electrode in an electrochemical sensor so that the sensor is linearly responsive over a large range of analyte concentrations and is easily calibrated. Mass transport limiting layers may include polymers and may be biocompatible. A mass transport limiting layer may provide many functions, e.g., biocompatibility and/or interferent-eliminating functions, etc. A mass transport limiting layer may be applied to an analyte sensor as described via any of a variety of suitable methods, including, e.g., dip coating and slot die coating, among others as would be understood by persons skilled in the art.

In certain embodiments, a mass transport limiting layer is a membrane composed of crosslinked polymers containing heterocyclic nitrogen groups, such as polymers of polyvinylpyridine and polyvinylimidazole. Embodiments also include membranes that are made of a polyurethane, or polyether urethane, or chemically related material, or membranes that are made of silicone, and the like.

A membrane may be formed by crosslinking in situ a polymer, modified with a zwitterionic moiety, a non-pyridine copolymer component, and optionally another moiety that is either hydrophilic or hydrophobic, and/or has other desirable properties, in an alcohol-buffer solution. The modified polymer may be made from a precursor polymer containing heterocyclic nitrogen groups. For example, a precursor polymer may be polyvinylpyridine or polyvinylimidazole. Optionally, hydrophilic or hydrophobic modifiers may be used to “fine-tune” the permeability of the resulting membrane to an analyte of interest. Optional hydrophilic modifiers, such as poly (ethylene glycol), hydroxyl or polyhydroxyl modifiers, may be used to enhance the biocompatibility of the polymer or the resulting membrane.

In some embodiments, a membrane may be formed in situ by applying an alcohol-buffer solution of a crosslinker and a modified polymer over the enzyme-containing sensing elements and allowing the solution to cure for a period of time, such as about one to two days or other appropriate period of time. The crosslinker-polymer solution may be applied over the sensing elements by placing a droplet or droplets of the membrane solution on the sensor, by dipping the sensor into the membrane solution, by spraying the membrane solution on the sensor, and the like. Generally, the thickness of the membrane is controlled by the concentration of the membrane solution, by the number of droplets of the membrane solution applied, by the number of times the sensor is dipped in the membrane solution, by the volume of membrane solution sprayed on the sensor, or by any combination of these factors. In order to coat the distal and side edges of the sensor, the membrane material may have to be applied subsequent to singulation of the sensor precursors. In some embodiments, the analyte sensor is dip-coated following singulation to apply one or more membranes. Alternatively, the analyte sensor could be slot-die coated wherein each side of the analyte sensor is coated separately. A membrane applied in the above manner may have any combination of the following functions: (1) mass transport limitation, i.e., reduction of the flux of analyte that can reach the sensing elements, (2) biocompatibility enhancement, or (3) interferent reduction.

In some embodiments, a membrane composition for use as a mass transport limiting layer may include one or more leveling agents, e.g., polydimethylsiloxane (PDMS). Additional information with respect to the use of leveling agents can be found, for example, in U.S. Pat. No. 8,983,568, filed Sep. 30, 2008 and entitled “Analyte Sensors Comprising Leveling Agents,” the disclosure of which is incorporated by reference herein in its entirety.

In some instances, the membrane may form one or more bonds with the sensing elements. The term “bonds” is intended to cover any type of an interaction between atoms or molecules that allows chemical compounds to form associations with each other, such as, but not limited to, covalent bonds, ionic bonds, dipole-dipole interactions, hydrogen bonds, London dispersion forces, and the like. For example, in situ polymerization of the membrane can form crosslinks between the polymers of the membrane and the polymers in the sensing elements. In certain embodiments, crosslinking of the membrane to the sensing element facilitates a reduction in the occurrence of delamination of the membrane from the sensor.

In some embodiments, analyte sensors as described herein may be factory calibrated and manufactured with minimal sensor-to-sensor variation. Calibration parameters from the factory calibration can be stored in the analyte monitoring assembly to allow for algorithmic correction to the measured analyte data. Analyte sensor can be worn for a predetermined period without the need for any user calibration. By reducing a profile of the sensor tail, and reducing foreign body response, analyte sensor can be worn for up to 14 days, 15 days, 28 days or 30 days or more, without the need for any user calibration. This feature differs from other existing sensors which require multiple fingerstick capillary blood glucose (BG) measurements for calibration.

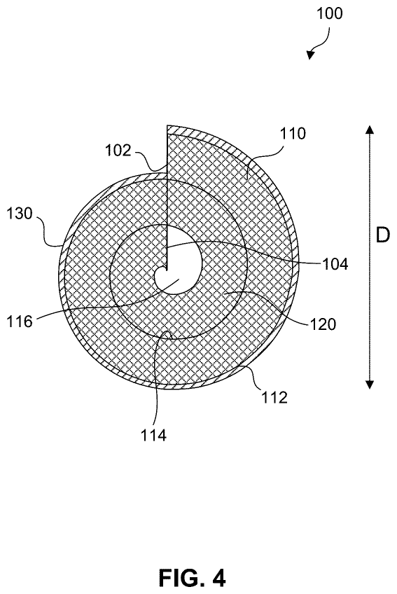

When in use to detect analytes, analyte sensor 100 is arranged in a generally tubular configuration, as shown for example in FIG. 3. Substrate layer 110 may be rolled or wound about a longitudinal axis X so that first side is brought toward second side. In some embodiments, a substrate layer 110 is wound so that electrodes 130 are arranged on an exterior surface 112 of substrate layer 110 so as to be exposed to bodily fluid and analytes (see, e.g., FIG. 4) when inserted into a body of a user. Electrodes 130 may be spaced from one another circumferentially on tubular substrate layer 110. In some embodiments, substrate layer 110 overlaps another portion of substrate layer 110. Substrate layer 110 is wound so that electrodes 130 are not overlapped by another portion of substrate layer 110 or by other electrodes 130.

In some embodiments, a transverse cross sectional area of analyte sensor 100 in a tubular configuration may be a circle, as best shown in FIG. 4. However, in some embodiments, the cross sectional area may be an oval or ellipse, among other shapes. In some embodiments, analyte sensor 100 may have a maximum diameter D of about 200 microns to 400 microns, 225 microns to 375 microns, or 250 to 350 microns, wherein the diameter is measured as the widest part of the transverse cross sectional area of analyte sensor 100 in the tubular configuration.

Core 120 of substrate layer 110 may be arranged within an interior area 116 defined by tubular substrate layer 110, as best shown in FIG. 4. Core 120 may serve to provide support to analyte sensor 100 in the tubular configuration. Core 120 may be arranged along and in facing engagement with an interior surface 114 of substrate layer 110. In some embodiments, substrate layer 110 may be wound to form two or more layers. In some embodiments, core 120 may partially or entirely fill interior area 116 of analyte sensor 100 when analyte sensor 100 is in the tubular configuration. Substrate layer 110 is wound so that core 120, and not electrodes 130, is placed in contact with interior surface 114 of substrate layer 110 such that electrodes 130 are exposed to a bodily fluid when analyte sensor 100 is inserted into a body of a user. In some embodiments, an adhesive, UV-curable resin, or the like, may be used to secure core 120 to interior surface 114 of substrate layer 110 in order to retain analyte sensor 100 in the tubular configuration.

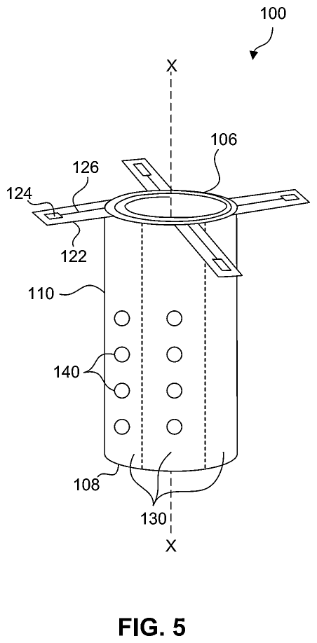

In some embodiments, extensions 122 of analyte sensor 100 may be positioned for connection to sensor electronics, as shown for example in FIG. 5. In use, analyte sensor 100 is inserted through the skin surface such that second end 108 of analyte sensor 100 is arranged below the skin surface and within the body of the user. First end 106 of analyte sensor 100 and extensions 122 with electrical contacts 124 are arranged outside of the body for electrical connection to sensor electronics of an analyte monitoring assembly as described in further detail herein.

Extensions 122 of substrate layer 110 may be parallel to a longitudinal axis of analyte sensor 100 when connected to sensor electronics. In some embodiments, however extensions 122 may be arranged at an angle to the longitudinal axis, and may be positioned transversely to longitudinal axis X. This may help to facilitate maintaining a small and compact analyte monitoring assembly. Extensions 122 may be arranged in a common plane. Extensions 122 may be folded or bent outwardly and away from central longitudinal axis X. Extensions 122 may extend radially outward from tubular substrate layer 110. In this way, each electrical contact 124 may be placed in electrical communication with sensor electronics of an analyte monitoring assembly, such as in electrical contact with a generally planar printed circuit board assembly. Sensor electronics may serve to receive signals from electrodes 130 and process and analyze the signals to determine analyte data, such as analyte levels.

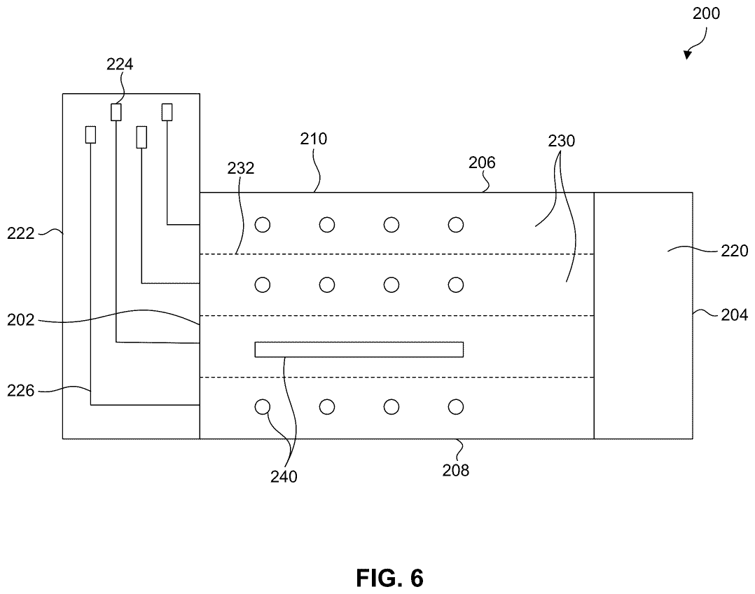

An in vivo analyte sensor according to an embodiment is shown in FIG. 6. Analyte sensor 200 may include a substrate layer 210 and electrodes 230 disposed thereon. Analyte sensor 200 includes the same features and construction as described above with respect to analyte sensor 100 except where specifically noted. Analyte sensor 200 differs primarily from analyte sensor 100 in the arrangement of electrodes 230. Electrodes 230 of analyte sensor 200 are arranged in a transverse direction of substrate layer 210 and extend from first side 202 to or toward opposing second side 204. Boundaries 232 between electrodes 230 may be arranged in a transverse direction (e.g., horizontal direction) and extend from first side 202 to or toward second side 204. Electrodes 230 of analyte sensor 200 are spaced from one another in a longitudinal direction of analyte sensor 200.

Similar to substrate layer 110, substrate layer 210 may include a core 220 at second side 204 of substrate layer 210, wherein there is no conductive material or electrodes 230 arranged on core 220. Alternatively, core 220 may be arranged at first side 202 of substrate layer 210.

As discussed above with respect to analyte sensor 100, analyte sensor 200 may include reagents 240 disposed on one or more of electrodes 230. Reagents 240 may be arranged in one or more locations on each electrode 230. Reagent 240 may be deposited on an electrode 230 in one or more spots. Spots may be arranged in a line, and may be spaced from one another at a fixed interval. In some embodiments, reagents 240 may be deposited on electrode 230 as a continuous line. In some embodiments, reagent 240 may include an analyte-responsive enzyme, as discussed in further detail herein.

Analyte sensor 200 may include an electrical contacts 224 in electrical communication with each electrode 230. Electrical communication between electrical contacts 224 and electrodes 230 may be made by electrical traces 226. Electrical contacts 224 and traces 226 may be arranged on one or more extensions 222 of substrate layer 210. In FIG. 6, a single extension 222 is shown that includes the electrical contacts 224 for each electrode 230. Extension 222 may extend from first side 202 of substrate layer 210, i.e., the side of substrate layer 210 opposite the core 220. Extension 222 may extend from second end 208 to and beyond first end 206.

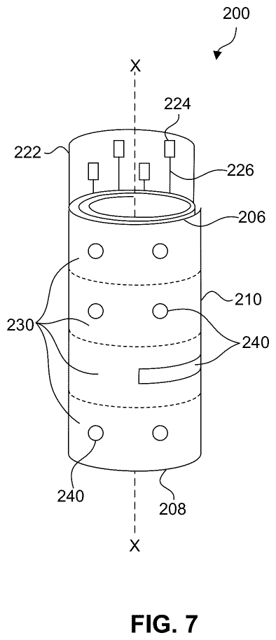

When analyte sensor 200 is in use, substrate layer 210 is arranged into a tubular configuration as shown for example in FIG. 7. Substrate layer 210 of analyte sensor 200 is wound around a longitudinal axis X. Each electrode 230 extends circumferentially around tubular substrate layer 210. Electrodes 230 are spaced from one another in a longitudinal direction of analyte sensor 200.

The longitudinal spacing of electrodes may facilitate application of membranes to analyte sensor 200. In some embodiments, a membrane may be applied to analyte sensor 200 by dipping analyte sensor 200 into a membrane solution. As electrodes are arranged longitudinally, one or more electrodes may be selectively coated in the membrane depending on how much of the analyte sensor is inserted into the membrane solution. For example, a first electrode 230 at second end 208 of analyte sensor 200 may be dipped into a first membrane solution while the remaining electrodes are not dipped in the membrane solution and are thus not coated with the first membrane. Analyte sensor 200 may then be dipped into a second membrane solution to cover the first electrode and a second electrode in the second membrane solution. Thus, the second electrode is coated only by the second membrane, and the first electrode is coated by both the first and second membranes. In this way, analyte sensor 200, having circumferentially arranged electrodes 230, facilitates coating of electrodes 230 by different combinations of membranes. Further, by arranging electrodes 230 along the longitudinal axis, different electrodes may be arranged at different depths within the body of the user as may be desired.

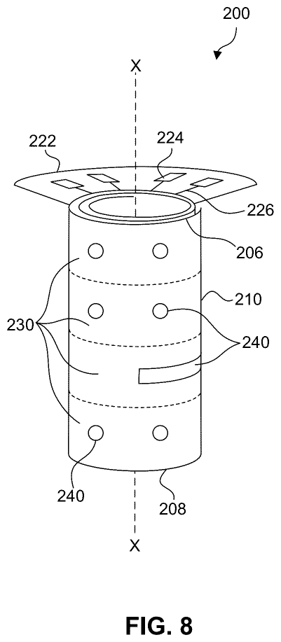

When analyte sensor 200 is part of an analyte monitoring assembly, extensions 222 may be positioned for contact with sensor electronics of an analyte monitoring assembly as shown for example in FIG. 8. As discussed with respect to analyte sensor 100 and FIG. 5, all or a portion of extension 222 may be folded or bent, such as into a plane transverse to a longitudinal axis of analyte sensor 200 to facilitate connection to sensor electronics. In some embodiments, a single extension 222 may be cut or otherwise divided into multiple extension portions, such that the individual extensions portions can be arranged in a desired position.

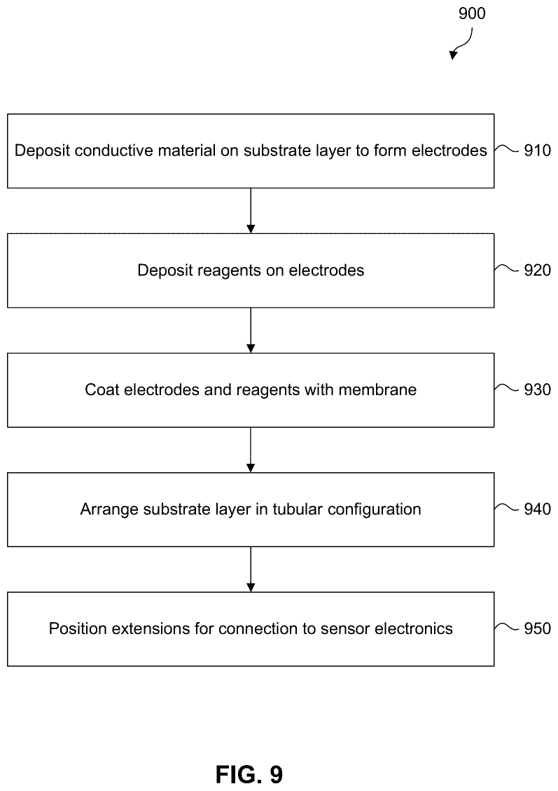

An exemplary method of forming an analyte sensor 900 is shown for example in FIG. 9. Electrodes are formed by depositing a conductive material on a substrate layer 910. If the substrate layer is coated with the conductive material, boundaries between electrodes may be formed by removing a portion of the conductive material, such as by laser ablation or etching. However, in alternate embodiments, conductive material may be selectively positioned to form separate electrodes separated by boundaries. Reagents 920 may be deposited on the electrodes for detecting analytes. Reagents may include, for example, an analyte-responsive enzyme. In some embodiments, reagents may be deposited prior to laser ablation or etching of the conductive material. The electrode and reagents may be coated by one or more membranes 930. The substrate layer may be arranged in a tubular configuration 940, such as by rolling or winding the substrate layer about a longitudinal axis. In some embodiments, membrane may be applied after the substrate layer is arranged in the tubular configuration. Extensions of substrate layer having electrical contacts may be positioned for connection to sensor electronics 950. In some embodiments, extensions may extend along a longitudinal axis of analyte sensor, or may be folded or bent such that they are arranged transversely or otherwise at an angle to longitudinal axis of analyte sensor.

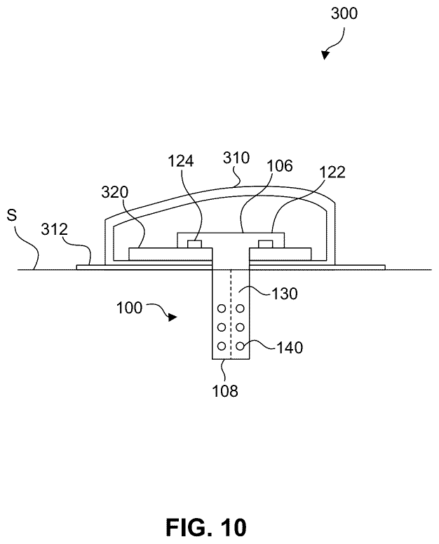

As shown in FIG. 10, an analyte sensor as described herein, e.g., analyte sensor 100, 200 may be used in an analyte monitoring assembly 300. Analyte monitoring assembly 300 may include a housing 310 configured to be mounted on a body of a user. Housing 310 may be arranged on a skin surface of the user. Housing 310 may include an adhesive patch 312 on a bottom surface of housing 310 for securing housing 310 to the skin surface S.

Housing 310 of analyte monitoring assembly 300 may contain sensor electronics 320. Sensor electronics 320 may be arranged outside of the body of the user. Sensor electronics 320 may be coupled to a portion of analyte sensor 100, 200. Specifically, sensor electronics 320 may be electrically connected to electrical contacts 124, 224 of extensions 122, 222 of analyte sensor 100, 200. Analyte sensor 100, 200 may include first end 106, 206 arranged within housing 310 and second end 108, 208 of analyte sensor 100, 200 may extend outwardly from housing 310 for positioning in the body of the user under the skin surface S and in contact with a bodily fluid, such as blood or interstitial fluid. Electrodes 130 or a portion thereof and reagents 140 may be arranged in the body of the user under the skin surface for detection of analytes in bodily fluid.

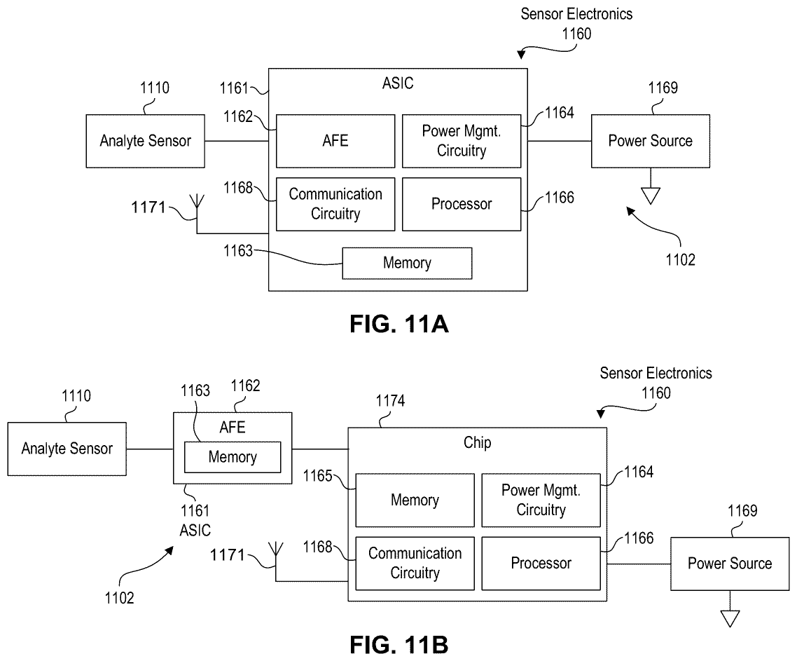

FIGS. 11A and 11B are block diagrams depicting example embodiments of analyte monitoring assemblies 1102 having analyte sensors 1110 as described herein and sensor electronics 1160. In some embodiments, sensor electronics 1160 (including analyte monitoring circuitry) that may have the majority of the processing capability for rendering end-result data suitable for display to the user. In FIG. 11A, a single semiconductor chip 1161 is depicted that can be a custom application specific integrated circuit (ASIC). Shown within ASIC 1161 are certain high-level functional units, including an analog front end (AFE) 1162, power management (or control) circuitry 1164, processor 1166, and communication circuitry 1168 (which can be implemented as a transmitter, receiver, transceiver, passive circuit, or otherwise according to the communication protocol). In this embodiment, both AFE 1162 and processor 1166 are used as analyte monitoring circuitry, but in other embodiments either circuit can perform the analyte monitoring function. Processor 1166 can include one or more processors, microprocessors, controllers, and/or microcontrollers, each of which can be a discrete chip or distributed amongst (and a portion of) a number of different chips.

A memory 1163 is also included within ASIC 1161 and can be shared by the various functional units present within ASIC 1161, or can be distributed amongst two or more of them. Memory 1163 can also be a separate chip. Memory 1163 can be volatile and/or non-volatile memory. In this embodiment, ASIC 1161 is coupled with power source 1169, which can be a coin cell battery, or the like. AFE 1162 interfaces with in vivo analyte sensor 1110 and receives measurement data therefrom and outputs the data to processor 1166 in digital form, which in turn processes the data to arrive at the end-result glucose discrete and trend values, etc. This data can then be provided to communication circuitry 1168 for sending, by way of antenna 1171, to a receiver device as described herein, for example, where minimal further processing is needed by the resident software application to display the data.

FIG. 11B is similar to FIG. 11A but instead includes two discrete semiconductor chips 1162 and 1174, which can be packaged together or separately. In FIG. 11B, AFE 1162 is resident on ASIC 1161. Processor 1166 is integrated with power management circuitry 1164 and communication circuitry 1168 on chip 1174. AFE 1162 includes memory 1163 and chip 1174 includes memory 1165, which can be isolated or distributed within. In one example embodiment, AFE 1162 is combined with power management circuitry 1164 and processor 1166 on one chip, while communication circuitry 1168 is on a separate chip. In another example embodiment, both AFE 1162 and communication circuitry 1168 are on one chip, and processor 1166 and power management circuitry 1164 are on another chip. It should be noted that other chip combinations are possible, including three or more chips, each bearing responsibility for the separate functions described, or sharing one or more functions for fail-safe redundancy.

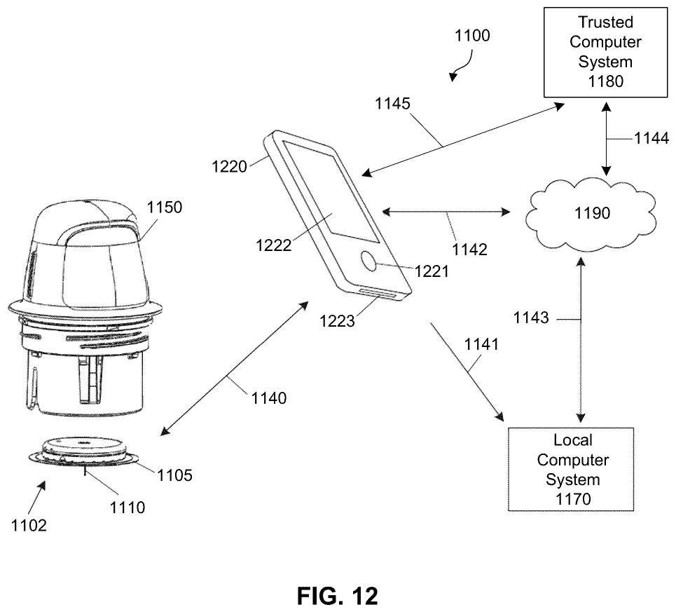

FIG. 12 is a conceptual diagram depicting an example embodiment of an analyte monitoring system 1100 that may incorporate one or more embodiments of the present disclosure. A variety of analytes can be detected and quantified using the system 100 (hereafter “the system 100”) including, but not limited to, alcohol, acetyl choline, amylase, bilirubin, cholesterol, chorionic gonadotropin, creatine kinase (e.g., CK-MB), creatine, DNA, fructosamine, glucose, glutamine, growth hormones, hormones, ketones (e.g., ketone bodies), lactate, oxygen, peroxide, prostate-specific antigen, prothrombin, RNA, thyroid stimulating hormone, and troponin. The concentration of drugs, such as, but not limited to, antibiotics (e.g., gentamicin, vancomycin, and the like), digitoxin, digoxin, drugs of abuse, theophylline, and warfarin, may also be determined.

Analyte monitoring system 1100 may include an analyte monitoring assembly 1102 as discussed herein (e.g., analyte monitoring assembly 300, 1400, 1800, 2000, 2100, 3200, 3402), among others. Analyte monitoring assembly may also be referred to as a sensor control device or “in vivo analyte sensor control device.” Analyte monitoring system 1100 may further include a receiver device 1220, also referred to as a “reader device,” in communication with the analyte monitoring assembly. Analyte monitoring assembly may include sensor electronics electrically coupled to an analyte sensor, such as an analyte sensor as described herein, and an adhesive layer for attachment to a skin surface of a user. Analyte monitoring assembly may further include a sensor applicator 1150, also referred to as an “inserter” configured to deliver the analyte monitoring assembly to a target monitoring location on the user's skin (e.g., the arm, abdomen, or buttocks of the user). Sensor applicator 1150 can be operated to position a portion of analyte sensor 1110 through the skin surface and into fluid contact with a bodily fluid. Sensor applicator 1150 can be used to place analyte monitoring assembly 1102 on a user's skin where a sensor 1110 is maintained in position for a period of time by an adhesive patch 1105. Adhesive patch may be coupled to analyte monitoring assembly, such as to a bottom of a housing of analyte monitoring assembly. A portion of an analyte sensor extends from the analyte monitoring assembly and is positioned such that it can be arranged transcutaneously or otherwise retained under the surface of the user's skin during the monitoring period. In some embodiments, applicator 1150 may position sensor electronics and/or adhesive patch 1105 on the skin surface. In some embodiments, applicator 1150 may be configured to secure sensor electronics to analyte sensor 1110. In some embodiments, sensor electronics, analyte sensor 1110 and adhesive patch 1105 may be contained or sealed within applicator 1150 prior to use. Applicator 1150 may be configured for use with insertion devices as described herein.

An introducer may be included to promote introduction of sensor 1110 into tissue. The introducer may comprise, for example, a needle often referred to as a “sharp.” Alternatively, the introducer may comprise other types of devices, such as a sheath or a blade. The introducer may temporarily reside in proximity to analyte sensor 1110 prior to tissue insertion and then be withdrawn after insertion of the analyte sensor 1110 into the tissue. While present, the introducer may facilitate insertion of analyte sensor 1110 into tissue by opening an access pathway for analyte sensor 1110 to follow. For example, the introducer may penetrate the epidermis to provide an access pathway to the dermis to allow subcutaneous implantation of analyte sensor 1110. After opening the access pathway, the introducer may be withdrawn (retracted) so that it does not represent a hazard while analyte sensor 1110 remains in place. In illustrative embodiments, the introducer may be solid or hollow, beveled or non-beveled, and/or circular or non-circular in cross-section.

In some embodiments, a tip of the introducer (while present) may be angled over the terminus of analyte sensor 1110, such that the introducer penetrates a tissue first and opens an access pathway for analyte sensor 1110. In other illustrative embodiments, analyte sensor 1110 may reside within a lumen or groove of the introducer, with the introducer similarly opening an access pathway for analyte sensor 1110. In either case, the introducer is subsequently withdrawn after facilitating analyte sensor 1110 insertion. Moreover, the introducer can be made of a variety of materials, such as various types of metals and plastics.

When analyte monitoring assembly is properly assembled, analyte sensor 1110 is placed in communication (e.g., electrical, mechanical, etc.) with one or more electrical components or sensor electronics included within analyte monitoring assembly 1102. In some applications, for example, analyte monitoring assembly 1102 may include a printed circuit board (PCB) having a data processor (e.g., an application specific integrated circuit or ASIC) mounted thereto, and analyte sensor 1110 may be operatively coupled to the data processor which, in turn, may be coupled with a memory, communication circuitry, such as an antenna, and a power source.

Analyte monitoring assembly 1102 can communicate with a receiver device 1220, also referred to herein as a reader device, via a communication path 1140 using a wired or wireless technique. The communication path 1140 may be uni- or bi-directional and may be encrypted or non-encrypted. Example wireless protocols include Bluetooth®, Bluetooth® Low Energy (BLE, BTLE, Bluetooth SMART, etc.), Near Field Communication (NFC), radio frequency identification (RFID), Wi-Fi, among other communication protocols and methods. Users can monitor applications installed in memory on receiver device 1220 using display 1222 and input component 1221, and the device battery can be recharged using power port 1223. Receiver device 1220 may include a touch-screen interface for use as input component 1221 and display 1222. While only one receiver device 1220 is shown, analyte monitoring assembly 1102 can communicate with multiple receiver devices 1220. Each of the receiver devices 1220 can communicate and share data with one another.

Receiver device 1220 can communicate with local computer system 1170 or trusted computer system 1180, also referred to as a remote terminal via a communication path 1141, 1145 using a wired or wireless communication protocol, which may also be wired or wireless, uni- or bi-directional, and encrypted or non-encrypted. Local computer system 1170 can include one or more of a laptop, desktop, tablet, smartphone, set-top box, video game console, or other computing device and wireless communication can include any of a number of applicable wireless networking protocols including Bluetooth, Bluetooth Low Energy (BTLE), Wi-Fi or others. Local computer system 1170 can communicate via communications path 1143 with a network 1190, such as a mobile telephone network, the internet, or a cloud server, similar to how receiver device 1220 can communicate via a communications path 1142 with network 1190, by a wired or wireless communication protocol as described previously. Network 1190 can be any of a number of networks, such as private networks and public networks, local area or wide area networks, and so forth. A trusted computer system 1180 can include one or more servers and can provide authentication services and secured data storage and can communicate via communications path 1144 with network 1190 by wired or wireless technique. Alternatively, analyte monitoring assembly 1102 may communicate directly with local computer system 1170 and/or trusted computer system 1180 without an intervening receiver device 1220 being present. For example, analyte monitoring assembly 1102 may communicate with local computer system 1170 and/or trusted computer system 1180 through a direct communication link to network 1190, according to some embodiments, as described in U.S. Pat. No. 10,136,816, filed Aug. 31, 2010, entitled “Medical Devices and Methods,” which is incorporated herein by reference in its entirety. Local computer system 1170 and/or trusted computer system 1180 may be accessible, according to some embodiments, by individuals other than a primary user who have an interest in the user's analyte levels, such as a parent, caregiver, or healthcare professional.

Information may be communicated from analyte monitoring assembly 1102 to receiver device 1220 automatically and/or continuously when the analyte information is available, or may not be communicated automatically and/or continuously, but rather stored or logged in a memory of analyte monitoring assembly 1102, e.g., for later output.

Data can be sent from sensor electronics to receiver device 1220 at the initiative of either analyte monitoring assembly 1102 or receiver device 1220. For example, in many example embodiments sensor electronics of analyte monitoring assembly 1102 can communicate data periodically in an unprompted or broadcast-type fashion, such that an eligible receiver device 1220, if in range and in a listening state, can receive the communicated data (e.g., sensed analyte data). This is at the initiative of sensor electronics because receiver device 1220 does not have to send a request or other transmission that first prompts sensor electronics to communicate. Broadcasts can be performed, for example, using an active Wi-Fi, Bluetooth, or BTLE connection, among others. The broadcasts can occur according to a schedule that is programmed within sensor electronics (e.g., about every 1 minute, about every 5 minutes, about every 10 minutes, or the like). Broadcasts can also occur in a random or pseudorandom fashion, such as whenever sensor electronics detects a change in the sensed analyte data. Further, broadcasts can occur in a repeated fashion regardless of whether each broadcast is actually received by a receiver device 1220.

Analyte monitoring system 1100 can also be configured such that receiver device 1220 sends a transmission that prompts analyte monitoring assembly 1102 to communicate its data to receiver device 1220. This is generally referred to as “on-demand” data transfer. An on-demand data transfer can be initiated based on a schedule stored in the memory of receiver device 1220, or at the behest of the user via a user interface of receiver device 1220. For example, if the user wants to check his or her analyte level, the user could perform a scan of sensor electronics using a near-field communication (NFC), Bluetooth®, BTLE, or Wi-Fi connection. Data exchange can be accomplished using broadcasts only, on-demand transfers only, or any combination thereof.

Analyte monitoring assembly 1102 may communicate with receiver device 1220 in a non-automatic manner and not according to a set schedule. For example, data may be communicated from analyte monitoring assembly 1102 using RFID technology when the sensor electronics are brought into communication range of receiver device 1220. Until communicated to receiver device 1220, data may remain stored in a memory of analyte monitoring assembly 1102. Thus, a patient does not have to maintain close proximity to receiver device 1220 at all times, and can instead upload data when convenient. In yet other embodiments, a combination of automatic and non-automatic data transfer may be implemented. For example, data transfer may continue on an automatic basis until receiver device 1220 is no longer in communication range of analyte monitoring assembly 1102.

Once analyte monitoring assembly 1102 is placed on the body so that at least a portion of analyte sensor 1110 is in contact with the bodily fluid and electrically coupled to the electronics within analyte monitoring assembly, sensor derived analyte information may be communicated in on-demand or unprompted (broadcast) fashion from the sensor electronics to a receiver device 1220. On-demand transfer can occur by first powering on receiver device 1220 (or it may be continually powered) and executing a software algorithm stored in and accessed from a memory of receiver device 1220 to generate one or more requests, commands, control signals, or data packets to send to sensor electronics. The software algorithm executed under, for example, the control of processing hardware of receiver device 1220 may include routines to detect the position of the analyte monitoring assembly 1102 relative to receiver device 1220 to initiate the transmission of the generated request command, control signal and/or data packet.

In some embodiments, receiver device 1220 may include, for example, a dedicated handheld receiver, PDA, laptop, computer, mobile telephone (e.g., smartphone), smartwatch, or other mobile electronic device or wearable electronic device. In some embodiments, sensor electronics may communicate with multiple receiver devices simultaneously or serially. Receiver device 1220 may be configured to receive analyte data from analyte monitoring assembly 1102 and to perform additional processing and analysis to generate and display analyte metrics, reports, to detect errors in operation of the analyte monitoring system 1100, and to generate and display alarms or notifications (e.g., high or low analyte level alarms, analyte rate of change alarms, and alarms related to errors in operation of the analyte monitoring assembly).

Analyte monitoring assembly 1102 may be included with sensor applicator 1150 in what is known as a “two-piece” architecture that requires final assembly by a user before analyte sensor 1110 can be properly delivered to the target monitoring location. More specifically, analyte sensor 1110 and the associated electrical components included in analyte monitoring assembly 1102 are provided to the user in multiple (two) packages, and the user must open the packaging and follow instructions to manually assemble the components before delivering analyte sensor 1110 to the target monitoring location with sensor applicator 1150.

Alternatively, analyte monitoring assembly 1102 and sensor applicator 1150 may have a “one-piece” architecture that allows the system to be shipped to the user in a single, sealed package that does not require any final user assembly steps. The user need only open one package and subsequently deliver analyte monitoring assembly 1102 to the target monitoring location. The one-piece system architecture may prove advantageous in eliminating component parts, various fabrication process steps, and user assembly steps. As a result, packaging and waste are reduced, and the potential for user error or contamination to the system is mitigated.

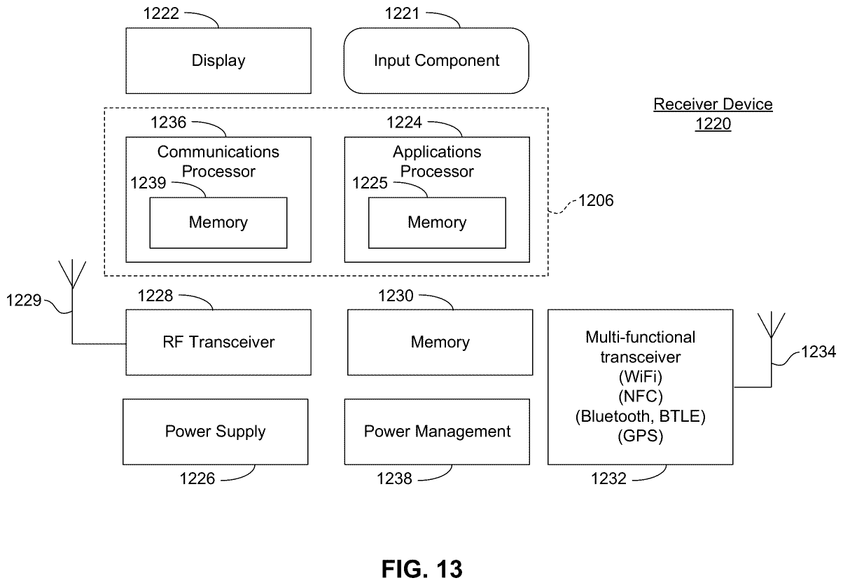

A block diagram depicting an example embodiment of a receiver device in an embodiment is shown in FIG. 13. Receiver device 1220 can include a display 1222 and an input component 1221, such as one or more keys, buttons, or a touch screen, among others. Receiver device 1220 may include one or more processors coupled to memory. Receiver device 1220 may include a processing core 1206 including one or both of a communications processor 1236 coupled with memory 1239 or an applications processor 1224 coupled with memory 1225. Also included can be separate memory 1230, RF transceiver 1228 with antenna 1229, and power supply 1226 with power management module 1238. Further, receiver device 1220 can also include a multi-functional transceiver 1232 which can communicate over Wi-Fi, NFC, Bluetooth®, BTLE, and GPS with an antenna 1234. As understood by one of skill in the art, these components are electrically and communicatively coupled in a manner to make a functional device.

In some embodiments, analyte monitoring assembly may have multiple sensor tails (or portions inserted under a skin surface of a user). Sensor tail may include an elongated structure. Sensor tail may be generally planar. Sensor tail may be configured as an elongated strip or wire. The analyte monitoring assembly may include an analyte sensor having a body and a plurality of sensor tails extending therefrom. The analyte sensor is configured to be coupled to sensor electronics. Analyte sensor and sensor electronics may be at least partially arranged within a housing. Each sensor tail may include one or more electrodes. The one or more electrodes may include a working electrode, a counter electrode, or a reference electrode. The working electrode may include an active area or sensing element as described herein. Sensor tails may be used to detect different analytes or the same analyte. An insertion device may be configured to facilitate insertion of the analyte sensor, and the sensor tails, into a body of a user for monitoring an analyte in a bodily fluid. The insertion device may include a body and a plurality of needles. Insertion device may include a needle corresponding to each sensor tail.

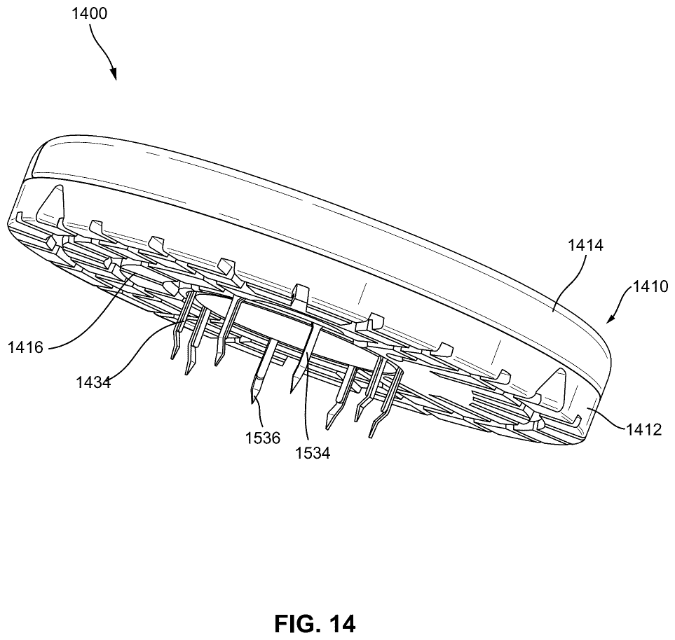

An analyte monitoring assembly having multiple sensor tails according to an embodiment is shown in FIG. 14. Analyte monitoring assembly 1400 is configured to be worn on a body of a user and may be mounted on a skin surface of the user, such as on an abdomen, buttocks, or arm, among other locations. Analyte monitoring assembly 1400 may include a plurality of sensor tails 1434 extending from housing 1410 for detecting a plurality of analytes in the bodily fluid. Each sensor tail 1434 may measure a different analyte. However, in some embodiments, two or more sensor tails 1434 may measure the same analyte. Further, in some embodiments, a first sensor tail 1434 may include a working electrode and a second sensor tail 1434 may include the corresponding counter electrode, such that the first and second sensor tails 1434 in combination can be used to detect a presence of an analyte. Each sensor tail 1434 may have one or more electrodes, and in some embodiments may have one to four electrodes. The one or more electrodes may include one or more of a working electrode, a counter electrode and a reference electrode.

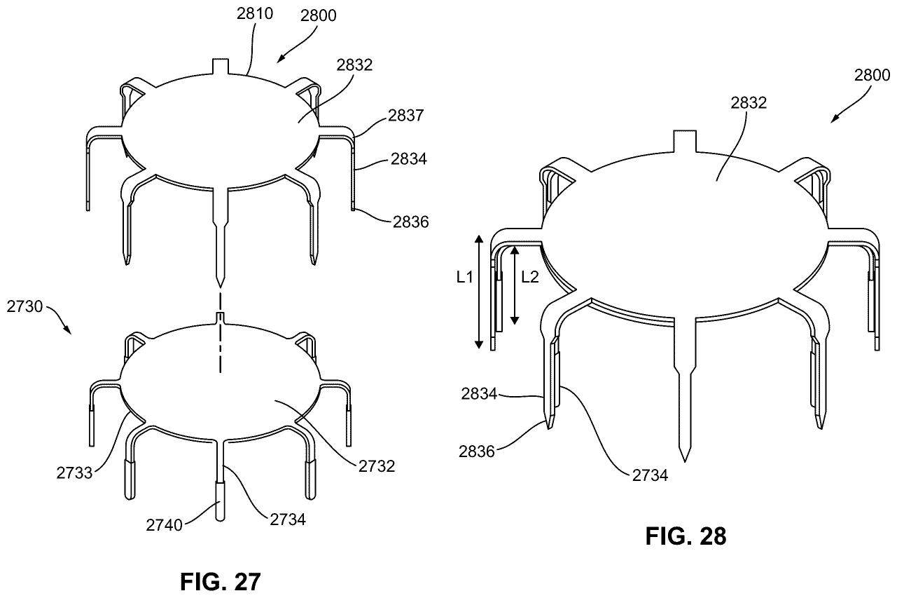

An insertion device having a plurality of needles 1534 may be used to pierce the skin of the user and to facilitate insertion of sensor tails 1434 into the skin, as discussed in further detail with respect to FIGS. 27-29. Needle 1534 may have a sharp, pointed tip 1536 to pierce the skin. Each sensor tail 1434 may have a corresponding needle 1534 for insertion of that sensor tail 1434 under the skin. Needles 1534 may extend generally perpendicularly from housing 1410 and may be configured to be inserted into the skin perpendicular to the skin surface. However, in some embodiments, needles 1534 may be configured for insertion into the skin at an angle relative to the skin surface. Needles 1534 may be part of an insertion device that is separate from analyte monitoring assembly 1400 and is used to insert sensor tails 1434 into skin and subsequently needles 1534 are removed from the skin leaving sensor tails 1434 in place and the insertion device is separated from analyte monitoring assembly 1400. In some embodiments, needles 1534 may be integrally formed with analyte monitoring assembly 1400 and may be retractable into housing 1410.

Analyte monitoring assembly 1400 may include a housing 1410 for supporting one or more analyte sensors and sensor electronics. In some embodiments, housing 1410 may have a circular shape in a top-down view, i.e., as viewed along a longitudinal axis of housing 1410. In some embodiments, housing 1410 may have alternative shapes, such as an oval, ellipse, square, rectangle, or oblong shape, among others. In some embodiments, housing 1410 may include a first or lower housing 1412 and a second or upper housing 1414 configured to be coupled to form housing 1410. First housing 1412 may include a base 1416 having one or more openings through which sensor tails 1434 and needles 1534 may extend.

Sensor electronics (including analyte monitoring circuitry) may be in electrical communication with the analyte sensor or sensors to receive signals indicative of analytes from the analyte sensor and to process the signals and generate analyte data, such as analyte concentrations and analyte trend or rate of change information, among other data. Sensor electronics may include a substrate, such as a printed circuit board, and one or more electronic components thereon. Electronic components may include one or more of a processor or processors, a memory coupled to the processor(s), such as a volatile or non-volatile memory, a power source, such as one or more batteries, power management circuitry, additional sensors, such as a temperature sensor, accelerometer, or gyroscope, among others, communication circuitry, such as a transmitter, transceiver, receiver, or antenna, for uni-directional or bi-directional communication and/or receiving data via a wireless communication protocol, among other components. Communication circuitry may include wireless communication circuitry including an antenna or multi-functional antenna, such as a Bluetooth® or Bluetooth® Low Energy (BLE) antenna, an NFC antenna, an RFID antenna, an RF transceiver, among other communication components. Sensor electronics may be configured to communicate analyte data and other information to a receiver device by wired or wireless communication. Sensor electronics may include a custom application specific integrated circuit (ASIC), and ASIC may include one or more of analog front end circuitry (AFE), processors, memory, and communication circuitry as discussed herein.

Sensor electronics or a portion thereof may be removably coupled to housing 1410 of analyte monitoring assembly 1400, such that sensor electronics are disposable or replaceable. For example, in some embodiments, a transmitter, power source, or both may be removably coupled to housing 1410 of analyte monitoring assembly 1400. When coupled to housing 1410, sensor electronics may be placed in electrical communication with the analyte sensor of analyte monitoring assembly 1400. However, in some embodiments, sensor electronics may be permanently coupled to housing 1410 of analyte monitoring assembly 1400.

Sensor electronics may receive analyte data from each sensor tail 1434 at the same or at different measurement intervals. In some embodiments, analyte monitoring assembly 1400 may receive measurements indicative of a first analyte at a first measurement interval and may collect measurements indicative of a second analyte at a second measurement interval that is different from the first measurement interval. Sensor electronics may be configured to transmit analyte data to a receiver device in communication with sensor electronics at the same or at different communication intervals. Sensor electronics may transmit analyte data indicative of first analyte to a receiver device at a first communication interval and may transmit analyte data indicative of a second analyte to a receiver device at a second communication interval that differs from the first communication interval. In some embodiments, sensor electronics may transmit analyte data indicative of first and second analytes to receiver device at the same time, such as in the same data packet.

As discussed in further detail herein, a receiver device or reader device may include, for example, a dedicated handheld receiver, PDA, laptop, computer, mobile telephone (e.g., smartphone), watch, or other mobile electronic device or wearable electronic device. In some embodiments, sensor electronics may communicate with multiple receiver devices simultaneously or serially. In some embodiments, receiver device may include a display, an input component (e.g., keyboard, buttons, touchscreen), one or more processors, memory coupled to the processor(s), a power supply with power management module, and communication circuitry, such as an RF transceiver and antenna. Receiver device may include a multi-functional transceiver that can communicate over one or more of Wi-Fi, NFC, Bluetooth®, Bluetooth® Low Energy (BLE). Receiver device may be configured to receive analyte data from analyte monitoring assembly and to perform additional processing and analysis to generate and display analyte metrics, reports, to detect errors in operation of the analyte monitoring system, and to generate and display alarms or notifications (e.g., high or low analyte level alarms, analyte rate of change alarms, and alarms related to errors in operation of the analyte monitoring assembly).

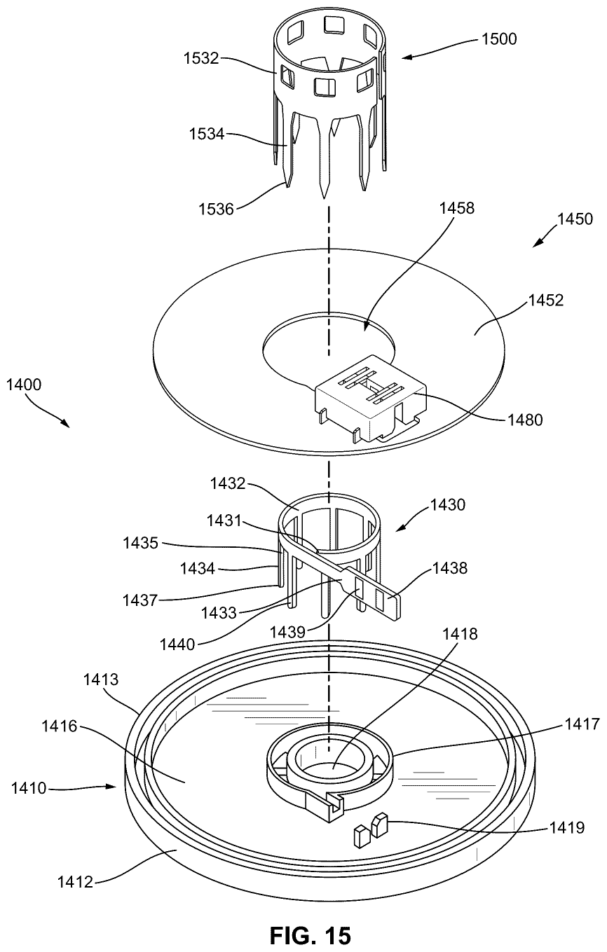

FIGS. 15-16 show a portion of an analyte monitoring assembly according to an embodiment. Analyte monitoring assembly 1400 includes a housing 1410 that supports sensor electronics 1450 and analyte sensor 1430. Housing 1410 may include a first housing 1412 and a second housing coupled together to enclose sensor electronics and at least a portion of analyte sensor 1430. First housing 1412 may include a base 1416 and an upstanding wall 1413 arranged around a perimeter of base 1416. Housing 1410 may include one or more support structures 1417, 1419 for supporting analyte sensor 1430. In some embodiments, support structure 1417, 1419 may include a channel configured to receive a portion of analyte sensor 1430. Openings in base 1416 may be arranged within channel 1417 such that when body 1432 of analyte sensor 1430 is arranged within channel 1417, sensor tails 1434 extend out of housing 1410 through the openings in base 1416 of housing 1410. Channel 1417 may be shaped to correspond to the shape of a body 1432 of analyte sensor 1430. Thus, if body 1432 has a ring shape, channel 1417 may similarly be ring-shaped. Housing 1410 may further include a support structure 1419 extending from base 1416 at a location spaced from channel 1417 for supporting a contact portion 1438 of analyte sensor 1430. First housing 1412 may further define an opening 1418, such as a central opening. Opening 1418 may provide ventilation and provide a path for egress of moisture from under an adhesive patch of the analyte monitoring assembly.

Sensor electronics 1450 may be arranged within housing 1410 on base 1416 of housing 1410. Sensor electronics 1450 may include a substrate 1452, such as a printed circuit board. Substrate 1452 may have a shape that corresponds to a shape of base 1416 of housing 1410. In some embodiments, substrate 1452 may have a generally annular configuration. Substrate 1452 may have a C-shape, or may occupy only a portion of housing 1410. Substrate 1452 may support analyte sensor 1430 and components of sensor electronics of analyte monitoring assembly 1400. Substrate 1452 may define an opening 1458 such that sensor tails 1434 may pass through opening 1458 and extend outwardly from housing 1410. Opening 1458 may be arranged centrally on substrate 1452. A connection device 1480 may be used to place analyte sensor 1430 in electrical communication with sensor electronics and to support analyte sensor 1430. In other embodiments, analyte sensor 1430 may be placed in communication with sensor electronics without use of an additional component, such as connection device 1480.

Analyte monitoring assembly 1400 includes an analyte sensor 1430 configured to detect a plurality of analytes in a bodily fluid. Analyte sensor 1430 includes a body 1432 having a plurality of sensor tails 1434 extending therefrom. Body 1432 and sensor tails 1434 may be integrally formed. Body 1432 of analyte sensor 1430 may be an elongated strip and includes a first end 1431 opposite a second end 1433. Body 1432 may include a contact portion 1438 having one or more electrical contacts 1439 configured to place analyte sensor 1430 in electrical communication with sensor electronics, either directly or via an intermediate component, such as connection device 1480.

Each sensor tail 1434 may be substantially linear in configuration and may have a planar or cylindrical configuration. Each sensor tail 1434 may include a first end 1435 opposite a second end 1437. One or more sensor tails 1434 may include one or more electrodes and/or an active area 1440. In some embodiments, sensor tail 1434 may be arranged coplanar with body 1432 of analyte sensor 1430. In some embodiments, sensor tail 1434 may be arranged at an angle to body 1432 of analyte sensor 1430 and may be arranged in a plane perpendicular to a plane of body 1432. Analyte sensor 1430 may have a curved or arcuate configuration. Body 1432 of analyte sensor 1430 may be curved so as to form a circle or ring shape, as shown in FIGS. 15-16. Analyte sensor 1430 may be curved such that first end 1431 of body 1432 may be arranged adjacent to body 1432 at or adjacent to second end 1433, with contact portion 1438 extending away from the ring formed by body 1432.

In some embodiments, sensor tails 1434 of analyte sensor 1430 may be spaced from one another, and may be spaced at a fixed interval. In some embodiments, the interval may be about 1 mm to 10 mm. Each sensor tail 1434 may have the same length as measured in a longitudinal direction from a first end to an opposing second end of sensor tail 1434. In some embodiments, each sensor tail 1434 may have a length in a range of 1 mm to 4 mm, or 1.5 mm to 3.5 mm, or 1 mm to 2 mm. Sensor tail 1434 may have a maximum thickness in a range of 150 microns to 400 microns, wherein the thickness is measured in a transverse direction of the sensor tail. Sensor tail 1434 may have a circular cross sectional area, and the thickness is measured as the diameter of the circular cross section. In some embodiments, sensor tail 1434 may have a rectangular cross sectional area, and the thickness may be measured as the longer dimension of the rectangular cross sectional area. Accordingly, sensor tails 1434 are shorter, thinner, or shorter and thinner than many existing analyte sensors.

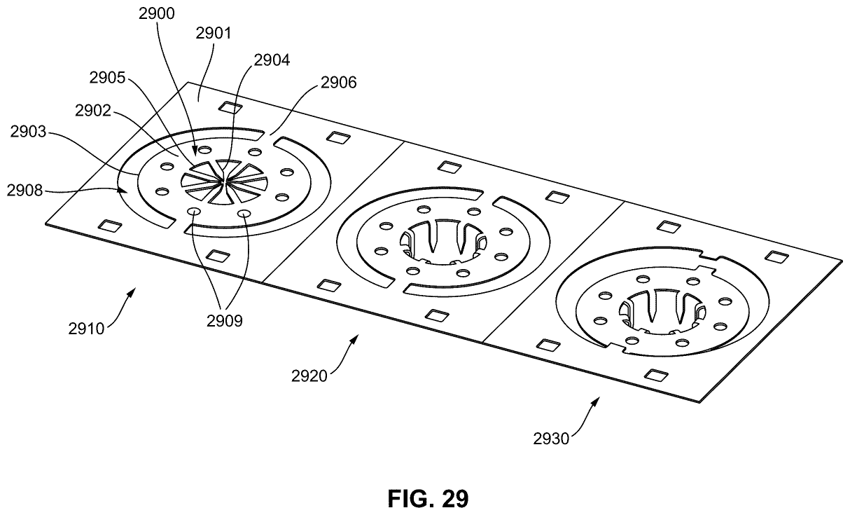

An insertion device 1500 may be used to pierce the skin of the user and to facilitate insertion of sensor tails 1434 in to the body of the user. Insertion device 1500 may include a plurality of needles 1534 extending from a body 1532. Body 1532 may be planar and needles 1534 may be arranged in a plane of body 1532. In some embodiments, body 1532 may be arranged at an angle to needles 1534, and may be arranged in a plane perpendicular to a plane of needles 1534. In some embodiments, body 1532 may include a curvature as shown in FIGS. 15-16. Body 1532 may be shaped in a circular or ring-shape. Insertion device 1500 may have a configuration corresponding to a configuration of analyte sensor 1430. For example, when analyte sensor 1430 is formed in a ring shape, insertion device 1500 may similarly be formed in a ring shape. Insertion device 1500 may be formed from a sheet of material, such as a metal or hard plastic, as discussed for example in connection with FIG. 29 below.

Insertion device 1500 may include a needle 1534 corresponding to each sensor tail 1434. Each needle 1534 may have a substantially linear configuration. However, in some embodiments, needle 1534 may have a stepped configuration as shown for example in FIG. 14 such that a tip of needle 1534 is aligned over a second end of sensor tail 1434. In some embodiments, needle 1534 has a flat, planar configuration or may have a curvature so as to partially surround a sensor tail 1434. Needle 1534 may include a pointed tip 1536 for piercing the skin. Needles 1534 may be spaced from one another on body 1532 at a fixed interval and may be generally parallel to one another. Each needle 1534 may have the same length. Needles 1534 are configured to be inserted through openings in base 1416 of housing 1410 along with sensor tails 1434. After sensor tails 1434 are inserted into a body of user, needles 1534 may be withdrawn from the body of the user leaving the sensor tails 1434 in place within the user's body.

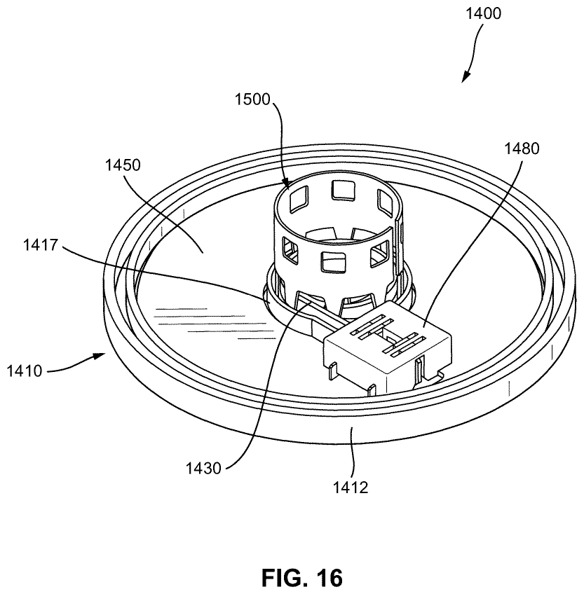

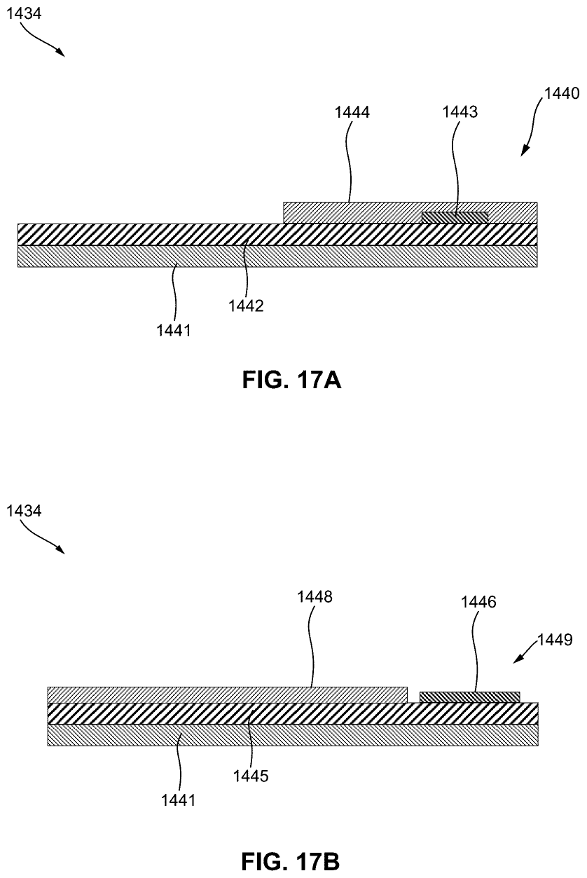

As shown in FIG. 17A, each sensor tail 1434 of the analyte sensor may include one or more electrodes 1442 to aid in detection of an analyte. Sensor tail may include a planar structure. For example, sensor tail may include a substrate with one or more electrodes and/or dielectric layers disposed thereon in a stacked arrangement. In some embodiments, sensor tail may have a cylindrical structure and may be formed as a wire with one or more electrodes and/or di-electric layers. Sensor tail may include a central wire with electrodes and/or di-electric layer arranged therearound in a nested or concentric configuration.

In some embodiments, each sensor tail 1434 may include a single electrode 1442 such that the size of sensor tail 1434 may be minimized. For example, a first sensor tail may include a single electrode that is a working electrode and a second sensor tail may include a single electrode that is a counter electrode. However, in some embodiments, sensor tail 1434 may include multiple electrodes, and each sensor tail may include one or more of a working electrode, a counter electrode, or a reference electrode, and may include multiple working electrodes, counter electrodes or reference electrodes. In some embodiments, each sensor tail 1434 may have one to four electrodes. Sensor tails having multiple electrodes may include a counter or reference electrode arranged on a same side of sensor tail 1434 as working electrode or on an opposing side. Additional electrodes may be electrically isolated from working electrode and from one another, such as by one or more dielectric layers. In some embodiments, a first sensor tail may include a working electrode, and a second sensor tail may include a counter electrode and/or reference electrode. A first sensor tail may include a first working electrode and a second sensor tail may include a second working electrode. First and second working electrodes may measure the same analyte for redundancy. Alternatively, first and second working electrodes may measure different analytes.

In some embodiments, a working electrode may include an active area for detection of an analyte. In some embodiments, working electrode may not include an active area and may be configured to measure a background signal or current. Measuring the background signal or noise can be used to correct an analyte level measured by a working electrode having an active area for detection of the analyte level. Multiple working electrodes may be present on one or more sensor tails and the working electrodes may have different electric potentials or poise voltages. In some embodiments, sensor tails may have different membranes or membrane assemblies.

FIGS. 17A and 17B show exemplary configurations of a sensor tail having one or more electrodes according to embodiments. FIG. 17A shows a sensor tail 1434 having a single working electrode 1442. Sensor tail 1434 includes a substrate 1441 having an electrode 1442 that serves as a working electrode. Working electrode 1442 may be in electrical communication with an electrical contact of analyte sensor via one or more electrical traces. Active area 1440 of sensor tail 1434 may include a reagent 1443 disposed on working electrode 1442. Reagent 1443 may be an analyte-responsive enzyme. Reagent 1443 may be applied on electrode as one or more spots. In some embodiments, sensor tail 1434 may include a single working electrode 1442 with an analyte-responsive enzyme thereon. In some embodiments, one or more membranes 1444, such as polymeric membranes, for biocompatibility, inhibiting interferents, or limiting analyte flux (e.g., mass transport limiting membrane) may be overcoated on all of a portion of sensor tail 1434.

In some embodiments, as shown in FIG. 17B, an analyte sensor may include a sensor tail 1434 having a reference electrode 1445. Reference electrode 1445 may include a carbon layer disposed on a substrate 1441 of sensor tail 1434. Sensor tail 1434 may further include a reference material layer 1446. Reference material layer 1446 may include silver (Ag) or silver chloride (AgCl) disposed on the reference electrode 1445. A dielectric layer 1448 may be disposed on reference material layer 1446. At least a portion of reference material layer 1446 is exposed to the bodily fluid via an opening 1449 in dielectric layer 1448. One or more membranes can be disposed over all of or a portion of sensor tail 1434. In such embodiments, a first sensor tail 1434 having a working electrode, as shown for example in FIG. 17A, may be used in combination with a sensor tail having a counter electrode or reference electrode to serve as an analyte sensing assembly.

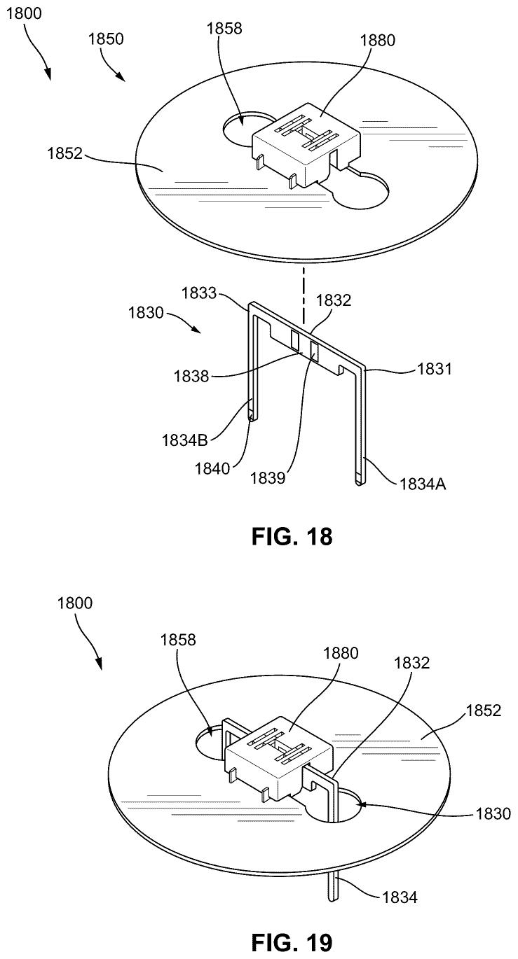

A portion of an analyte monitoring assembly 1800 according to an embodiment is shown in FIGS. 18 and 19. Analyte sensor 1830 may include a body 1832 and a plurality of sensor tails 1834A, 1834B extending from body 1832. In FIG. 18, analyte sensor 1830 includes a first sensor tail 1834A extending from a first end 1831 of body 1832 and a second sensor tail 1834B extending from an opposing second end 1833 of body 1832. Each sensor tail 1834 may include an active area 1840 configured to detect an analyte, as described for example with respect to FIGS. 17A and 17B. In some embodiments, each sensor tail 1834A, 1834B may detect the same analyte or may detect different analytes. In some embodiments, a first sensor tail 1834A may include a working electrode, and second sensor tail 1834B may include a counter electrode, a reference electrode, or both such that first and second sensor tails 1834A, 1834B can be used in combination to detect an analyte.

Sensor tails 1834 may be arranged substantially perpendicular to body 1832. Sensor tails 1834 may be integrally formed with body 1832. Sensor tails 1834 may be arranged substantially parallel to one another. Each sensor tail 1834 may have the same length or may have different lengths. Sensor tails 1834 may be spaced from one another, such as by a fixed distance, e.g., about 1 mm to 10 mm. In some embodiments, sensor tails 1834 and body 1832 are arranged in a single plane. However, as discussed above, in some embodiments, body 1832 may have a curvature, such as an arc or C-shape, among other shapes. In some embodiments, body 1832 may be arranged in a plane perpendicular to a plane of the sensor tails 1834A, 1834B, and parallel to a plane of substrate 1852.

Body 1832 may include a contact portion 1838 having one or more electrical contacts 1839. Electrical contacts 1839 may be in electrical communication with active area 1840 and electrodes thereon, such as by electrical traces. Contact portion 1838 may be configured to connect to sensor electronics to communicate signals detected by sensor tails 1834 to sensor electronics for processing. Contact portion 1838 may be arranged centrally on body 1832 and between first and second sensor tails 1834A, 1834B.

Analyte sensor 1830 may be coupled to sensor electronics 1850. Sensor electronics 1850 may include a substrate 1852, such as a printed circuit board. Analyte sensor 1830 may be arranged centrally with respect to substrate 1852 and may extend along a diameter of substrate 1852, as best shown in FIG. 19. Analyte sensor 1830 may be arranged generally perpendicularly to a plane of substrate 1852. Body 1832 of analyte sensor 1830 may be arranged on or just above plane of substrate 1852. In some embodiments, body 1832 may be parallel to substrate 1852.

In some embodiments, analyte sensor 1830 may be electrically connected to substrate 1852 via a connection device 1880. Connection device 1880 may be configured to electrically connect analyte sensor 1830 to substrate 1852. Connection device 1880 may be configured to electrically connect electrical contacts 1839 of analyte sensor 1830 to electrical connections on substrate 1852. Connection device 1880 may also serve to retain and secure analyte sensor 1830 in a desired position. Substrate 1852 may define one or more openings 1858 through which sensor tails 1834 of analyte sensor 1830 extend. In some embodiments, substrate 1852 may include one opening 1858 for each sensor tail 1834. For example, where analyte sensor 1830 has two sensor tails 1834A, 1834B, substrate 1852 may define two openings 1858. However, in some embodiments, one opening 1858 may accommodate multiple sensor tails 1834.

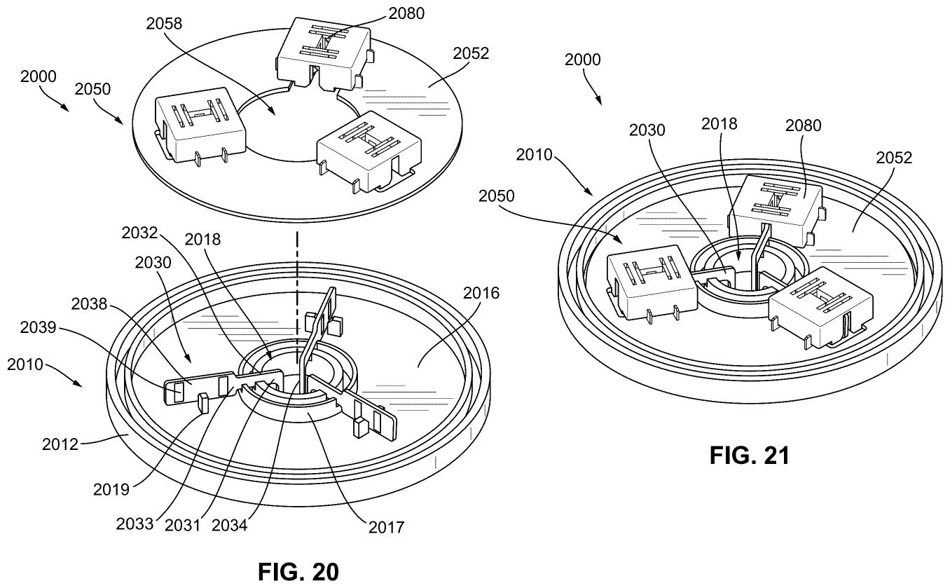

A portion of an analyte monitoring assembly 2000 according to some embodiments is shown in FIGS. 20 and 21. Similar to analyte monitoring assemblies 1400, 1800, analyte monitoring assembly 2000 includes a housing 2010, one or more analyte sensors 2030, and sensor electronics 2050. In FIGS. 20 and 21, analyte monitoring assembly 2000 includes three analyte sensors 2030. However, it is understood that in other embodiments, analyte monitoring assembly 2000 may include fewer or additional analyte sensors 2030. Each analyte sensor 2030 may include one or more sensor tails 2034 for detection of analytes. In some embodiments, each analyte sensor may detect a different analyte. However, in other embodiments, a first analyte sensor 2030 may have a sensor tail 2034 having a working electrode (e.g., FIG. 17A), and a second analyte sensor 2030 may have a sensor tail 2034 having a counter or reference electrode (e.g., FIG. 17B), such that the first and second analyte sensors can be used in combination to detect an analyte. Using multiple separate analyte sensors 2030 may facilitate manufacturing of analyte monitoring assembly, as a first analyte sensor may include sensor tails to be manufactured by similar processes (e.g., dipping or coating of the sensor tail with different reagents, membranes, or coatings), whereas a second analyte sensor may include sensor tails that require different reagents, membranes, or coatings.

Each analyte sensor 2030 may have a body 2032 and one or more sensor tails 2034 extending from body 2032. In FIGS. 20 and 21, each analyte sensor 2030 has a single tail 2034. However, in alternate embodiments, analyte sensors 2030 may each have multiple sensor tails 2034. Body 2032 may include a first end 2031 opposite a second end 2033. Sensor tail 2034 may extend from first end 2031 of body 2032. Each sensor tail 2034 may include one or more electrodes as discussed above for example with respect to FIGS. 17A and 17B. In some embodiments, each sensor tail 2034 may include an active area for detecting an analyte as described herein. Sensor tail 2034 may be arranged substantially perpendicularly to body 2032 such that analyte sensor 2030 has an L-shaped configuration. Body 2032 may include a contact portion 2038 having one or more electrical contacts 2039 for connection of analyte sensor 2030 to sensor electronics. Contact portion 2038 may be arranged at second end 2033 of body 2032. Contact portion 2038 is in electrical communication with active area of sensor tail 2034 for receiving signals detected by sensor tail 2034.

Each analyte sensor 2030 may be arranged radially with respect to housing 2010 and substrate 2052 of sensor electronics 2050 on base 2016 of first housing 2012. Analyte sensors 2030 may be evenly spaced and may be symmetrically arranged with respect to housing 2010. For example, where three analyte sensors 2030 are present, the analyte sensors 2030 may be spaced by about 120°. Each sensor tail 2034 may extend through an opening 2058 of substrate 2052 and an opening 2018 of housing 2010. Opening 2018 may be arranged centrally on housing 2010. As shown in FIGS. 20-21 each sensor tail 2034 extends through a single opening 2018 in housing 2010. Housing 2010 may further include one or more support structures 2017, 2019 for securing analyte sensors 2030 in place. Each analyte sensor 2030 may be connected to substrate 2052 via a connection device 2080 as described with respect to connection devices 1480, 1880.

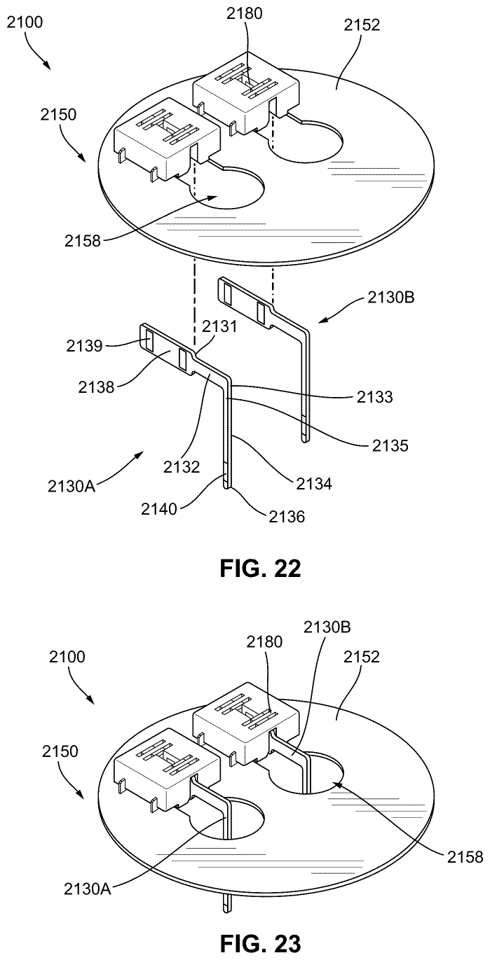

A portion of an analyte monitoring assembly according to some embodiments is shown in FIGS. 22 and 23. Similar to analyte monitoring assemblies 1400, 1800, 2000, analyte monitoring assembly 2100 includes a housing, sensor electronics 2150, and one or more analyte sensors 2130A, 2130B. Similar to analyte monitoring assembly 2000, analyte monitoring assembly 2100 includes a plurality of analyte sensors 2130A, 2130B. Each analyte sensor 2130A, 2130B has a body 2132 and one or more sensor tails 2134 extending from body 2132. Body 2132 may have a first end 2131 opposite a second end 2133. Sensor tail 2134 may extend from first end 2131 of body 2132. Sensor tail 2134 may include a first end 2135 opposite a second end 2136, with first end 2135 connected to body 2132 and second end 2136 extending away from body 2132. Thus, analyte sensor 2130 may have a substantially L-shaped configuration. Body 2132 and sensor tail 2134 may be arranged in a single plane. In some embodiments, body 2132 may have a curvature, or body 2132 may be arranged in a plane perpendicular to plane of sensor tail 2134 such that body 2132 is parallel to substrate 2152. Second end 2136 of sensor tail 2134 may include one or more electrodes to aid in detection of an analyte. One or more sensor tails 2134 may include an active area 2140 for detecting an analyte. Body 2132 may include contact portion 2138 having one or more electrical contacts 2139 for electrical connection of analyte sensor 2130 to sensor electronics 2150 of analyte monitoring assembly 2100. Electrical contacts 2139 may be electrically connected to electrodes, such as by electrical traces, to communicate signals indicative of analyte levels to sensor electronics. In some embodiments, first sensor tail 2134 of first analyte sensor 2130A may include a working electrode, and second sensor tail of analyte sensor 2130B may include a counter and/or reference electrode, such that first and second sensor tails 2134 may be used in combination to detect an analyte. However, in other embodiments, each sensor tail 2134 may include a working electrode for detecting an analyte.

Substrate 2152 of sensor electronics 2150 may define multiple openings 2158 through which sensor tails 2134 may extend. In FIGS. 22 and 23, substrate 2152 includes a first opening 2158 for receiving sensor tail 2134 of first analyte sensor 2130A, and a second opening 2158 for receiving sensor tail 2134 of second analyte sensor 2130B. Analyte sensor 2130 may be electrically connected to substrate 2152 via connection device 2180. In some embodiments, each analyte sensor 2130 has a separate connection device 2180.

Analyte sensors 2130A, 2130B may be spaced from one another, such as at a fixed distance of about 1 mm to 10 mm. Analyte sensors 2130A, 2130B may be arranged generally parallel to one another as shown in FIGS. 22 and 23. However, in some embodiments, analyte sensors may be arranged collinearly or may be arranged at an angle to one another, such as perpendicularly.

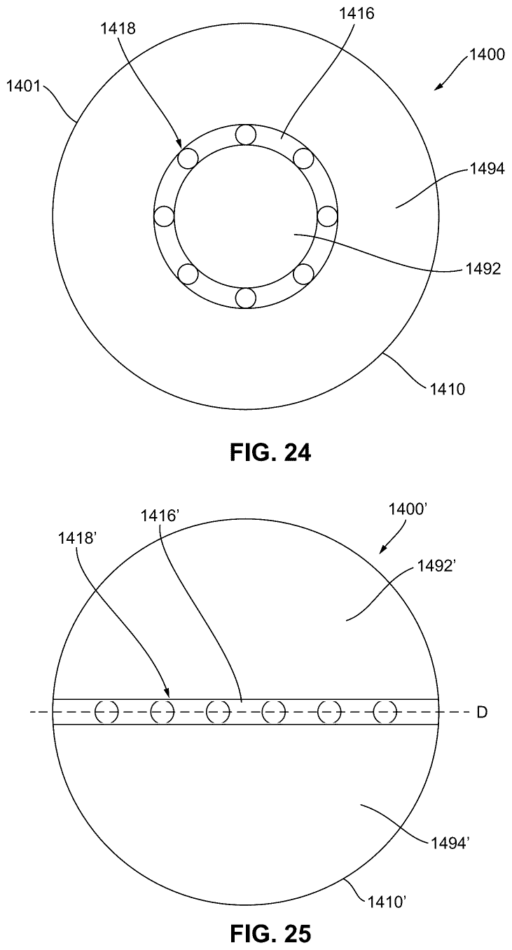

In any of the embodiments described herein, analyte monitoring assemblies 1400, 1800, 2000, 2100 may include one or more adhesive patches configured to adhere analyte monitoring assembly to a body of a user. Adhesive patch or patches may be arranged on an exterior surface of base 1416 of housing 1410. In some embodiments, openings 1418 on base 1416 of housing 1410, and thus sensor tails extending therethrough, are arranged in a ring shape as shown for example in FIG. 24. Openings 1418 may be arranged in a ring that is spaced inwardly from outer perimeter 1401 of housing 1410. In such embodiments, analyte monitoring assembly 1400 may include first adhesive patch 1492 arranged within an interior area defined by the ring of openings 1418 and a second adhesive patch 1494 arranged in an area exterior to the ring of openings 1418. First adhesive patch 1492 may have a circular shape and may substantially fill the interior area within the ring of openings 1418. Second adhesive patch 1494 may have an annular shape and may surround the ring of openings 1418. In some embodiments, second adhesive patch 1494 may be divided into two or more arc-shaped patches. For example, second adhesive patch 1494 may include two C-shaped patches that together form an annular shape. First and second adhesive patches 1492, 1494 may be spaced from one another so as to allow openings 1418 to remain uncovered.

In some embodiments, analyte monitoring assembly 1400′ may include openings 1418′ and sensor tails arranged in a line, as shown for example in FIG. 25. Base 1416′ of housing 1410′ may define openings 1418′ in a linear arrangement, such as along a diameter D of housing 1410′. Openings 1418′ may be evenly spaced from one another. In such embodiments, analyte monitoring assembly 1400′ may include a first adhesive patch 1492′ on a first side of line of openings 1418′ and a second adhesive patch 1494′ on a second, opposing side of line of openings 1418′. Each adhesive patch 1492′, 1494′ may have a substantially semi-circular shape. First and second adhesive patches 1492′, 1494′ may be separated by a space such that openings 1418′ are not covered.

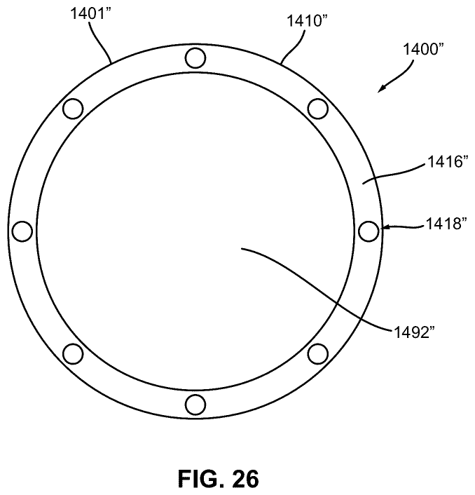

In some embodiments, analyte monitoring assembly 1400″ may include openings 1418″ arranged about a perimeter 1401″ of base 1416″ of housing 1410″ as shown in FIG. 26. Such embodiments may help to maximize spacing between openings 1418″ and thus the sensor tails. In such embodiments, analyte monitoring assembly 1400″ may include a single adhesive patch 1492″. Adhesive patch 1492″ may have a circular shape and may be placed centrally on base 1416″ of housing 1410″. Adhesive patch 1492″ may have a diameter that is less than a diameter of housing 1410″ so that adhesive patch 1492″ does not cover openings 1418″. Such embodiment may reduce the number of adhesive patches for adhering analyte monitoring assembly 1400″ to the user, and thus simplifies manufacturing. The single central adhesive patch 1492″ also provides a continuous uninterrupted area of adhesive.

Analyte sensors as described herein can be inserted into the body of the user with the assistance of an insertion device. An insertion device according to an embodiment is shown for example in FIGS. 27 and 28. As shown in FIGS. 27 and 28, insertion device 2800 includes a body 2832 and a plurality of needles 2834 extending therefrom. Insertion device 2800 may be part of an applicator configured to automate insertion of sensor into the body of the user. Applicator may be configured to supply a force to insert insertion device 2800 into the skin of the user and to secure the analyte monitoring assembly to the body of the user. Applicator may include a drive mechanism, such as a spring to provide the force to insert the insertion device, or the user may supply the force. Applicator may automatically retract insertion device from the body of the user and separate the insertion device from the analyte monitoring assembly.

In some embodiments, each needle 2834 of insertion device 2800 may have a first end 2837 connected to body 2832 and a second end 2836 extending away from body 2832. Second end 2836 of needle 2834 may be pointed to pierce skin of the user. In some embodiments, body 2832 and needles 2834 are integrally formed. In some embodiments, body 2832 is arranged in the same plane as needle 2834. As shown in FIG. 27, body 2832 is arranged in a plane that is perpendicular to a plane in which needles 2834 are arranged.

Insertion device 2800 may have a configuration corresponding to that of analyte sensor 2730. For example, in FIGS. 27 and 28, insertion device 2800 has a disk-shaped body and a plurality of needles 2834 extending from a perimeter 2810 of body 2832. Similarly, analyte sensor 2730 has a disk-shaped body 2732 with a plurality of sensor tails 2734 extending from a perimeter 2733 of body 2732, with each sensor tail 2734 having one or more electrodes as discussed for example with respect to FIGS. 17A-17B. One or more sensor tails 2734 may include an active area 2740 for detecting an analyte. Insertion device 2800 may include a needle 2834 corresponding to each sensor tail 2734. For example, in FIGS. 27 and 28, analyte sensor 2730 has eight sensor tails 2734 and insertion device 2800 has eight needles 2834.

As best shown in FIG. 28, when insertion device 2800 is positioned for inserting analyte sensor 2730 into a body of a user, insertion device 2800 is placed in an overlapping position with analyte sensor 2730. Body 2832 of insertion device 2800 is placed on top of body 2732 of analyte sensor 2730. Insertion device 2800 and analyte sensor 2730 may be placed in facing engagement. As shown in FIG. 28, a length L1 of needle 2834 may be the same as or greater than a length L2 of sensor tail 2734. In this way, needles 2834 may pierce skin and create a path through which sensor tails 2734 are guided into the body of the user. Sensor tails 2734 may lack sufficient strength or rigidity to pierce the user's skin and tissue, and thus needle 2834 may help to facilitate insertion and proper placement of sensor tail 2734. Needles 2834 may have a planar configuration with sensor tail 2734 being arranged adjacent an interior facing surface of needle 2834. In some embodiments, needles 2834 may form a channel, such as a C-shaped channel, in which the sensor tail 2734 can be arranged.

A method of manufacturing an insertion device for an analyte monitoring assembly according to an embodiment is shown in FIG. 29. An insertion device 2900 may include a body 2902 and a plurality of needles 2904 extending from body 2902. A base material 2901, such as a sheet of metal or hard plastic, may be provided. Base material 2901 may be processed, such as by etching, cutting, punching, or the like, to remove one or more regions 2908 of base material 2901 and to form a shape of the insertion device 2900, as shown in step 2910. In some embodiments, body 2902 may have an annular shape with an inner perimeter 2905 and an outer perimeter 2903, and the plurality of needles 2904 may extend radially from inner perimeter 2905 of body 2902 toward a center of insertion device 2900. Body 2902 may include one or more apertures 2909 therethrough. Apertures 2909 may help to precisely locate insertion device 2900 as needles 2904 are being formed. Apertures 2909 may further facilitate attachment of additional components to insertion device 2900, such as to secure insertion device 2900 to an applicator for applying an analyte monitoring assembly to a body of a user. Body 2902 may be connected to a remainder of base material 2901 by one or more contact portions 2906. After etching, the insertion device 2900 may be shaped into a desired configuration as shown in step 2920. Needles 2904 are bent from a position co-planar with body 2902 into a position in which needles 2904 are substantially perpendicular to body 2902. In some embodiments, forming insertion device may further include bending each needle into a staggered configuration (see, e.g., FIG. 14). Insertion device 2900 may be separated or singulated from a remainder of base material as shown in step 2930. This may be performed by cutting contact portions 2906 coupling insertion device 2900 to base material 2901. In some embodiments, additional finishing processes may be performed, so as to smooth the insertion device 2900 and/or to apply one or more coatings.

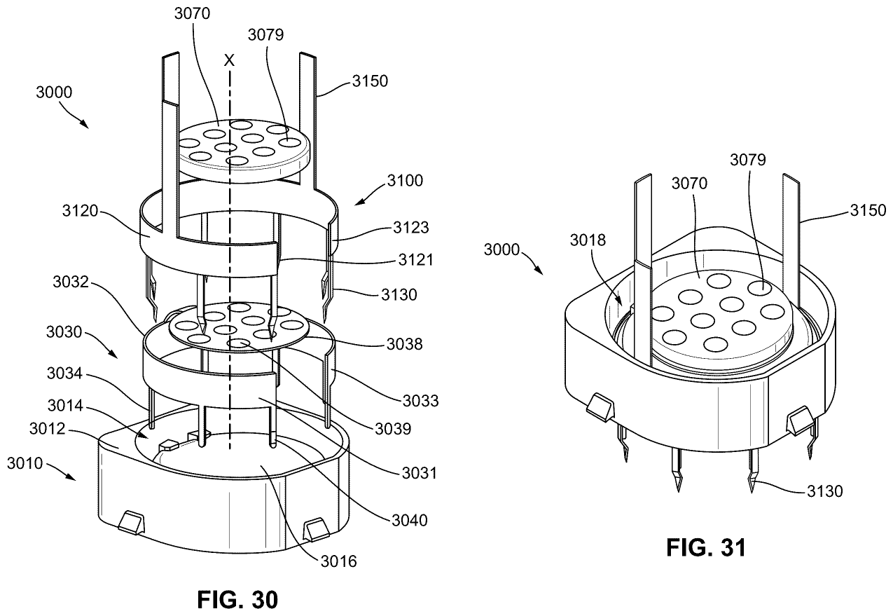

A plug assembly 3000 according to an embodiment is shown in FIGS. 30 and 31. The plug assembly 3000 includes a support 3010 configured to receive and support an analyte sensor 3030. Analyte sensor 3030 includes a body 3032 having a plurality of sensor tails 3034 extending therefrom. Sensor tails 3034 may be parallel to one another and may be spaced from one another at a fixed interval. Sensor tails 3034 may each have the same shape and dimensions. Sensor tails 3034 each include one or more electrodes to aid in detection of an analyte. One or more of sensor tails 3034 may include an active area 3040 for detecting an analyte. In some embodiments, one or more sensor tails 3034 may include a counter electrode, reference electrode or both. Body 3032 of analyte sensor 3030 may be arranged in a same plane as a plane of sensor tails 3034, or may be arranged at an angle thereto, and may be perpendicular to sensor tails 3034. Body 3032 of analyte sensor 3030 may be linear or may have a curvature. As shown in FIG. 30, body 3032 is curved about a central longitudinal axis X and is shaped as a portion of a circle in a top-down view. First end 3031 of body 3032 is shown as being spaced from second end 3033 by a gap such that sensor tails 3034 are spaced from one another. Analyte sensor 3030 further includes a contact portion 3038 extending from body 3032. Contact portion 3038 is perpendicular to sensor tails 3034 and is arranged in a plane transverse to longitudinal axis X of plug assembly 3000. Contact portion 3038 is shown formed as a flat plate or disk. Contact portion 3038 includes a plurality of electrical contacts 3039 for electrical connection to sensor electronics.

Plug assembly 3000 includes a support 3010 configured to receive and support analyte sensor 3030. Support 3010 may include a body 3012 having a central opening 3014 in which a platform 3016 is arranged. A channel 3018 (FIG. 31) may be defined between body 3012 and platform 3016. When analyte sensor 3030 is arranged on support 3010, body 3032 is arranged within channel 3018 with sensor tails 3034 extending outward and away from support 3010, such as through openings in a body 3012 of support 3010. Contact portion 3038 of analyte sensor 3030 may be arranged on platform 3016 and may be placed in facing engagement therewith.

In some embodiments, plug assembly 3000 includes a connector 3070. Connector 3070 may include electrical contacts 3079 configured to electrically connect to respective electrical contacts 3039 of analyte sensor 3030 and to sensor electronics. Connector 3070 may have a shape similar to the shape of contact portion 3038 of analyte sensor 3030. In FIGS. 30 and 31, connector 3070 is shown as a circular disk. Connector 3070 may serve as a seal and may be made of an elastomeric material, such as silicone, among others.

An insertion device 3100 may include a body 3120 and a plurality of needles 3130 extending from body 3120. Body 3120 and needles 3130 may be integrally formed. Body 3120 may include a first end 3121 opposite a second end 3123. Body 3120 may have a curvature, and may be shaped as a ring. Body 3120 may have a configuration similar to body 3032 of analyte sensor 3030. Insertion device 3100 may include a needle 3130 corresponding to each sensor tail 3034. In FIGS. 30 and 31, needles 3130 have a staggered configuration with a first portion configured to extend along and parallel to sensor tail 3034 and a second portion that extend beyond an end of sensor tail 3034 and that is arranged along the axis of the sensor tail 3034 to guide insertion of sensor tail 3034 into the skin. Insertion device 3100 may include one or more extensions 3150 extending from body 3120. Extensions 3150 may be elongated strips. Extensions 3150 may be substantially planar. Extensions 3150 are shown as having a generally rectangular shape. Extensions 3150 may be integrally formed with body 3120 and needles 3130. Extensions 3150 may extend from an opposing side of body 3120 as needles 3130 (e.g., needles may extend from a lower end of body and extensions may extend from an upper end of body). Extensions 3150 may be engaged by an applicator to facilitate movement of insertion device 3100 including withdrawal of needles 3130 from the skin after sensor tails 3034 have been placed under the skin, and withdrawal of insertion device 3100 from the analyte monitoring assembly.

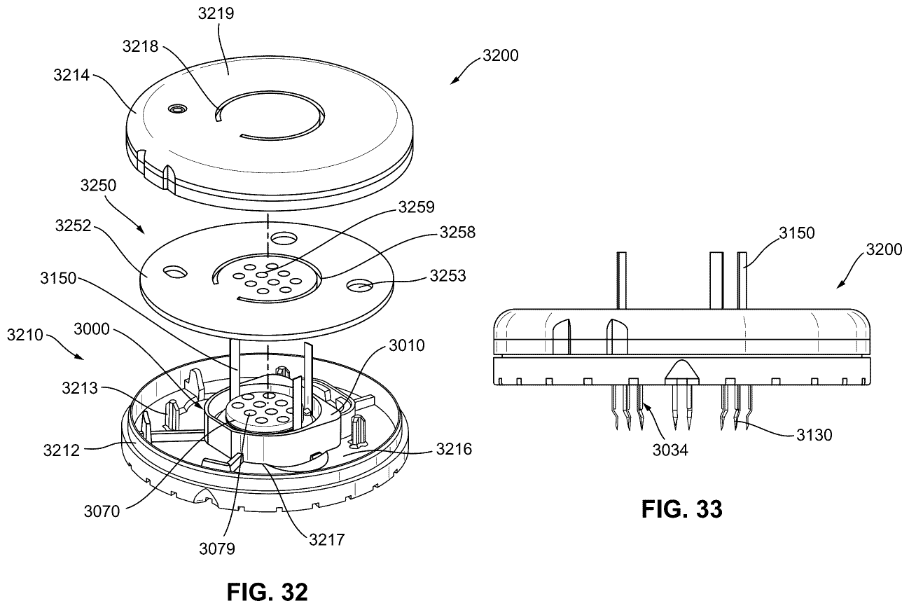

As shown in FIG. 32, an analyte monitoring assembly 3200 may include a housing 3210 having a first housing 3212 and a second housing 3214 (e.g., a lower housing and an upper housing) configured to be coupled together, as shown in FIG. 33. First housing 3212 may include a central opening 3217 in base 3216 to receive plug assembly 3000. Central opening 3217 may have a shape corresponding to a shape of support 3010 of plug assembly 3000.

Sensor electronics 3250 may be arranged within housing 3210. Sensor electronics 3250 may include a substrate 3252, such as a printed circuit board, arranged on first housing 3212. Substrate 3252 may include one or more electrical contacts 3259 configured to be placed in electrical communication with electrical contacts 3079 of connector 3070. Substrate 3252 may have a shape corresponding to a shape of first housing 3212. For example, in FIG. 32, first housing 3212 has a generally circular shape and thus substrate 3252 may similarly have a circular shape so as to be received within first housing 3212. Substrate 3252 may include a slot 3258 through which insertion device 3100 may extend. Slot 3258 may be shaped similarly to shape of body of insertion device 3100. Slot 3258 is shown as having a shape of a portion of a circle or C-shape. Substrate 3252 may further include apertures 3253 for receiving support structures, such as posts 3213, therethrough. In FIG. 32, first housing 3212 includes one or more posts 3213 configured to extend through apertures 3253 of substrate 3252 and engage with support structures on second housing 3214 to secure first and second housings together 3212, 3214 and to retain substrate 3252 in place.

Second housing 3214 may define a slot 3218 in its upper surface 3219. Slot 3218 may be shaped similarly to body of insertion device 3100. When analyte monitoring assembly 3200 is fully assembled, as shown in FIG. 33, extensions 3150 of insertion device 3100 extend outward from housing 3210 through slot 3218. In this way, an applicator can engage extensions 3150 to control insertion and retraction of needles 3130 of insertion device 3100. When sensor tails 3034 are inserted into the user, insertion device 3100 can be fully withdrawn from analyte monitoring assembly 3200 by passing through slot 3258 of substrate 1252 and through slot 3218 of second housing 3214.

Any of the analyte monitoring assemblies described herein may be part of an analyte monitoring system, as shown for example in FIG. 12, as discussed above.

In summary, analyte monitoring assemblies for monitoring analytes in a bodily fluid of a user include a housing having a base mountable to a body of a user. The assembly includes an analyte sensor having a body and a plurality of sensor tails extending therefrom. The body of the analyte sensor is arranged on the housing and the plurality of sensor tails extend out of the housing and into the body of the user. A first sensor tail includes a first active area for detecting signals indicative of a first analyte. A second sensor tail includes a second active area for detecting signals indicative of a second analyte. The assembly further includes sensor electronics arranged within the housing and coupled to the analyte sensor. The sensor electronics receive signals indicative of first and second analytes from the analyte sensors and wirelessly transmit analyte data to a receiver device.

Some embodiments describe herein relate to an analyte sensor having a reduced width sensor tail. Analyte sensor may include a tail portion connected to a flag portion by an intermediate portion. The tail portion may include a substrate. A conductive layer may be disposed on a surface of the substrate to form an electrode. A reagent may be disposed on the conductive layer for detection of an analyte (e.g., an active area or sensing element as described herein). Tail portion may be coated in a membrane. A width of sensor tail measured from a first side to an opposing second side of the sensor tail in a direction traverse to a longitudinal axis of the sensor tail may be reduced by cutting sensor tail in a longitudinal direction along one or both of first and second sides of sensor tail. Sensor tail may be cut or otherwise reduced in width so that a width of sensor tail is the same as or slightly greater than a width of the reagent or active area of sensor tail.

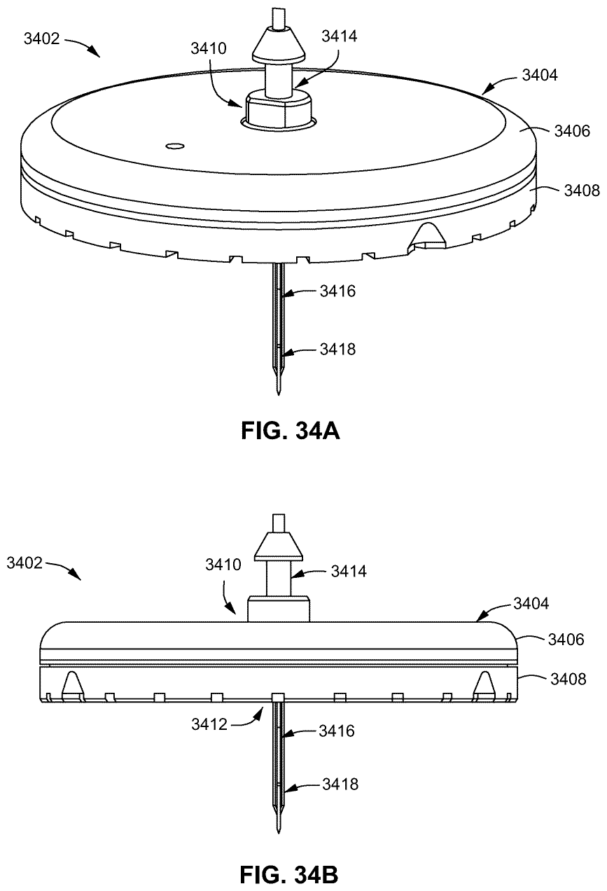

FIGS. 34A and 34B are perspective and side views, respectively, of an exemplary sensor control device 3402, according to one or more embodiments of the present disclosure. Sensor control device 3402 may be similar in some respects to analyte monitoring assembly 1102 of FIG. 12 and therefore may be best understood with reference thereto. Sensor control device 3402 may replace analyte monitoring assembly 1102 of FIG. 12 and, therefore, may be used in conjunction with sensor applicator 1150 (FIG. 12), which delivers sensor control device 3402 to a target monitoring location on a user's skin.

As illustrated, sensor control device 3402 includes an electronics housing 3404 that is generally disc-shaped and may have a circular cross-section. In other embodiments, however, electronics housing 3404 may exhibit other cross-sectional shapes, such as ovoid (e.g., pill-shaped), a squircle, or polygonal, without departing from the scope of the disclosure. Electronics housing 3404 may be configured to house or otherwise contain various electrical components used to operate sensor control device 3402.

Electronics housing 3404 may include a first housing or shell 3406 and a second housing or mount 3408 that is matable with shell 3406. Shell 3406 may be secured to mount 3408 via a variety of ways, such as a snap fit engagement, an interference fit, sonic welding, or one or more mechanical fasteners (e.g., screws). In some cases, shell 3406 may be secured to mount 3408 such that a sealed interface therebetween is generated. In such embodiments, a gasket or other type of seal material may be positioned at or near the outer diameter (periphery) of shell 3406 and mount 3408, and securing the two components together may compress the gasket and thereby generate a sealed interface. In other embodiments, an adhesive may be applied to the outer diameter (periphery) of one or both of shell 3406 and mount 3408. The adhesive secures shell 3406 to mount 3408 and provides structural integrity, but may also seal the interface between the two components and thereby isolate the interior of the electronics housing 3404 from outside contamination. If sensor control device 3402 is assembled in a controlled environment, there may be no need to terminally sterilize the internal electrical components. Rather, the adhesive coupling may provide a sufficient sterile barrier for assembled electronics housing 3404.

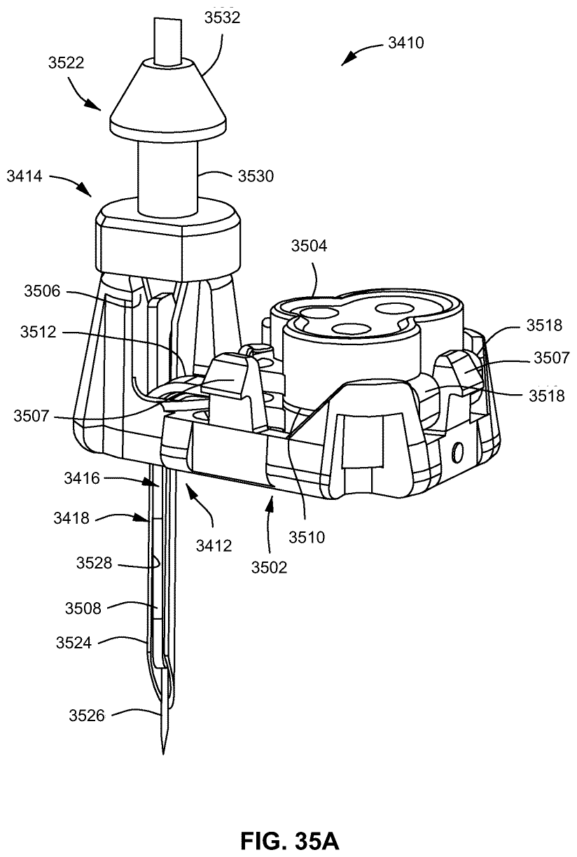

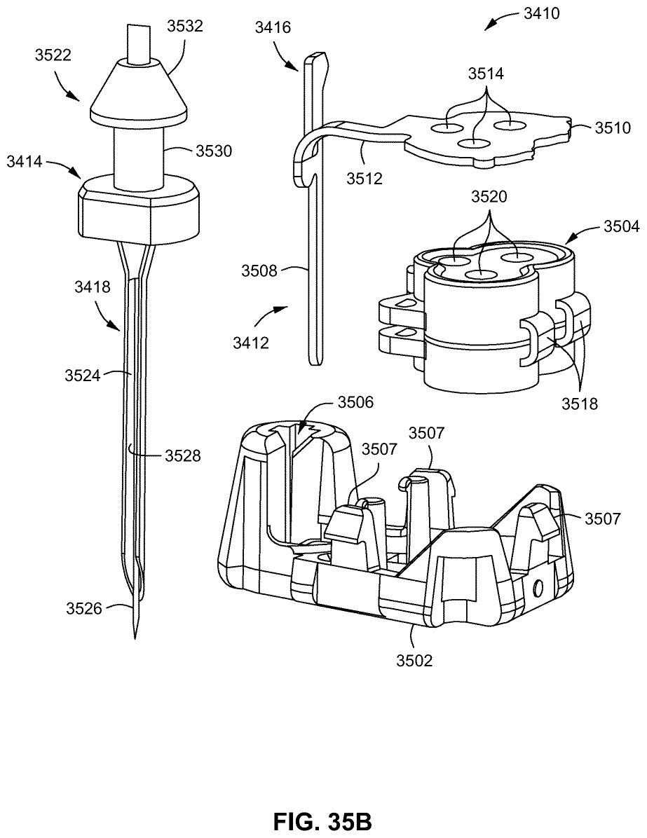

Sensor control device 3402 may further include a plug assembly 3410 (as shown for example in FIG. 35A) that may be coupled to electronics housing 3404. For example, plug assembly 3410 may include a sensor module 3412 interconnectable with a sharp module 3414. Sensor module 3412 may be configured to carry and otherwise include an analyte sensor 3416, and sharp module 3414 may be configured to carry and otherwise include an introducer, such as sharp 3418, used to help deliver analyte sensor 3416 transcutaneously under a user's skin during application of sensor control device 3402. As illustrated, corresponding portions of analyte sensor 3416 and sharp 3418 extend from electronics housing 3404 and, more particularly, from the bottom of mount 3408. The exposed portion of analyte sensor 3416 may be received within a hollow or recessed portion of sharp 3418. The remaining portion of analyte sensor 3416 is positioned within the interior of electronics housing 3404.

FIGS. 35A and 35B are isometric and exploded views, respectively, of plug assembly 3410, according to one or more embodiments. Sensor module 3412 may include analyte sensor 3416, a plug 3502, and a connector 3504. Plug 3502 may be designed to receive and support both analyte sensor 3416 and connector 3504. As illustrated, a channel 3506 may be defined through plug 3502 to receive a portion of analyte sensor 3416. Moreover, plug 3502 may provide one or more deflectable arms 3507 configured to snap into corresponding features provided on a bottom of electronics housing 3404 (see, e.g., FIGS. 34A-34B).

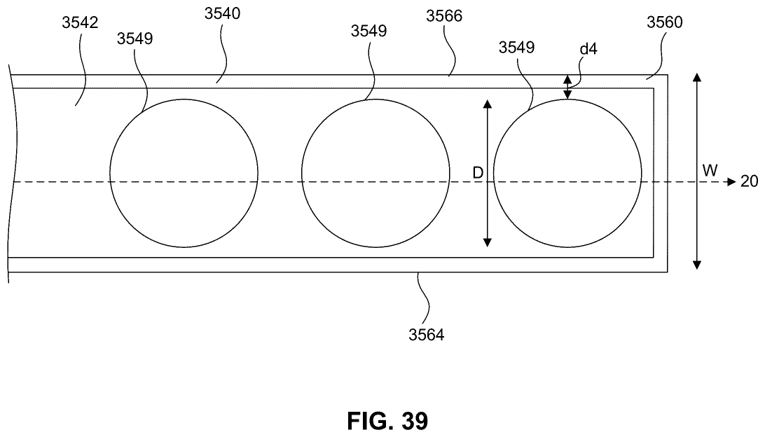

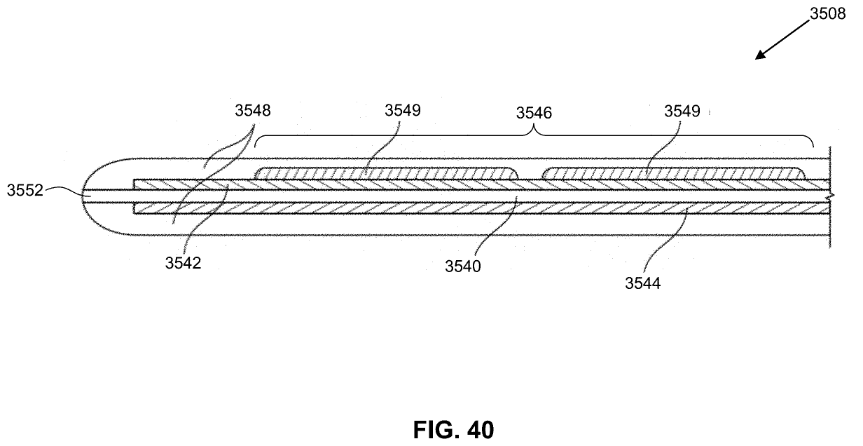

In some embodiments, analyte sensor 3416 includes a tail 3508, a flag 3510, and a neck 3512 that interconnects tail 3508 and flag 3510. Neck 3512 can be bent approximately 90 degrees such that flag 3510 is in a plane perpendicular to a plane of tail 3508. Tail 3508 may be configured to extend at least partially through channel 3506 and extend distally from plug 3502. Tail 3508 includes active area having sensing chemistry, which may include an analyte-responsive enzyme, among other chemistry that is reactive to target analyte in bodily fluid (see, e.g., FIGS. 39 and 40). In some embodiments, a membrane 3548 may cover at least active area 3546 of tail 3508. In use, the tail 3508 is transcutaneously received beneath a user's skin, such that active area 3546 is in contact with a bodily fluid for detecting the target analyte.

Flag 3510 may comprise a generally planar surface. Flag 3510 may have one or more sensor contacts 3514 (three shown in FIG. 35B) arranged thereon. Sensor contact(s) 3514 may be configured to connect to sensor electronics to communicate analyte data or signals to sensor electronics. Sensor contacts 3514 may be configured to align with a corresponding number of compliant carbon impregnated polymer modules (not shown) encapsulated within connector 3504 of plug assembly 3410.

Connector 3504 may include one or more hinges 3518 that enables connector 3504 to move between open and closed states. Connector 3504 is depicted in FIGS. 35A-35B in the closed state, but can pivot to the open state to receive flag 3510 and the compliant carbon impregnated polymer module(s) therein. The compliant carbon impregnated polymer module(s) provide electrical contacts 3520 (three shown) configured to provide conductive communication between analyte sensor 3416 and corresponding circuitry contacts provided within electronics housing 3404 (see, e.g., FIGS. 34A-34B). Connector 3504 can be made of silicone rubber and may serve as a moisture barrier for analyte sensor 3416 when assembled in a compressed state and after application to a user's skin.

Sharp module 3414 includes sharp 3418 and a sharp hub 3522 that carries sharp 3418. Sharp 3418 includes an elongate shaft 3524 and a sharp tip 3526 at the distal end of shaft 3524. Shaft 3524 may be configured to extend through channel 3506 and extend distally from plug 3502. Moreover, shaft 3524 may include a hollow or recessed portion 3528 that at least partially circumscribes tail 3508 of analyte sensor 3416. Sharp tip 3526 may be configured to penetrate the skin while carrying tail 3508 to put the active area of tail 3508 into contact with bodily fluids.

Sharp hub 3522 may include a hub small cylinder 3530 and a hub snap pawl 3532, each of which may be configured to help couple plug assembly 3410 (and the entire sensor control device 3402) to sensor applicator 1150 (see, e.g., FIG. 12). Analyte sensor 3416 may include an active area as discussed herein.

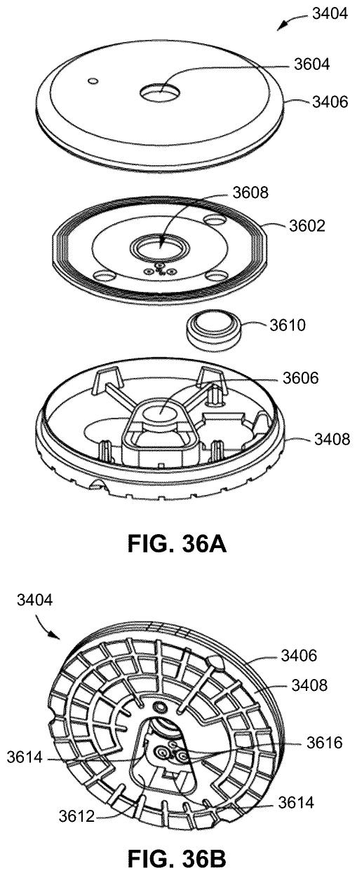

FIGS. 36A and 36B are exploded and bottom isometric views, respectively, of the electronics housing 3404, according to one or more embodiments. The shell 3406 and the mount 3408 operate as opposing clamshell halves that enclose or otherwise substantially encapsulate the various electronic components of the sensor control device 3402.

A printed circuit board (PCB) 3602 may be positioned within electronics housing 3404. A plurality of electronic modules may be mounted to PCB 3602 including, but not limited to, a data processing unit, resistors, transistors, capacitors, inductors, diodes, and switches. The data processing unit may comprise, for example, an application specific integrated circuit (ASIC) configured to implement one or more functions or routines associated with operation of sensor control device 3402. More specifically, the data processing unit may be configured to perform data processing functions, where such functions may include but are not limited to, filtering and encoding of data signals, each of which corresponds to a sampled analyte level of the user. The data processing unit may also include or otherwise communicate with an antenna for communicating with the receiver device 1220 (FIG. 12).

As illustrated, shell 3406, mount 3408, and PCB 3602 each define corresponding central apertures 3604, 3606, and 3608, respectively. When electronics housing 3404 is assembled, central apertures 3604, 3606, and 3608 coaxially align to receive plug assembly 3410 (FIGS. 35A-35B) therethrough. A battery 3610 may also be housed within the electronics housing 3404 and configured to power sensor control device 3402.

In FIG. 36B, a plug receptacle 3612 may be defined in mount 3408 and provides a location where plug assembly 3410 may be received and coupled to the electronics housing 3404, and thereby fully assemble sensor control device 3402. The profile of plug 3502 may match or be shaped in complementary fashion to plug receptacle 3612, and plug receptacle 3612 may provide one or more snap ledges 3614 configured to interface with and receive the deflectable arms 3507 of the plug 3502. Plug assembly 3410 is coupled to the electronics housing 3404 by advancing the plug 3502 into the plug receptacle 3612 and allowing the deflectable arms 3507 to lock into the corresponding snap ledges 3614. When the plug assembly 3410 is properly coupled to the electronics housing 3404, one or more circuitry contacts 3616 defined on the underside of the PCB 3602 may make conductive communication with electrical contacts 3520 of connector 3504.

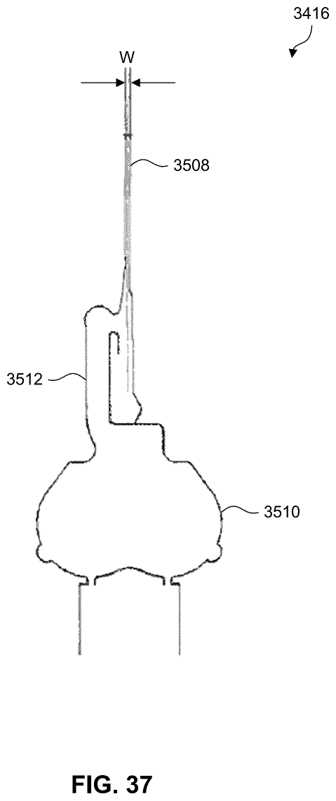

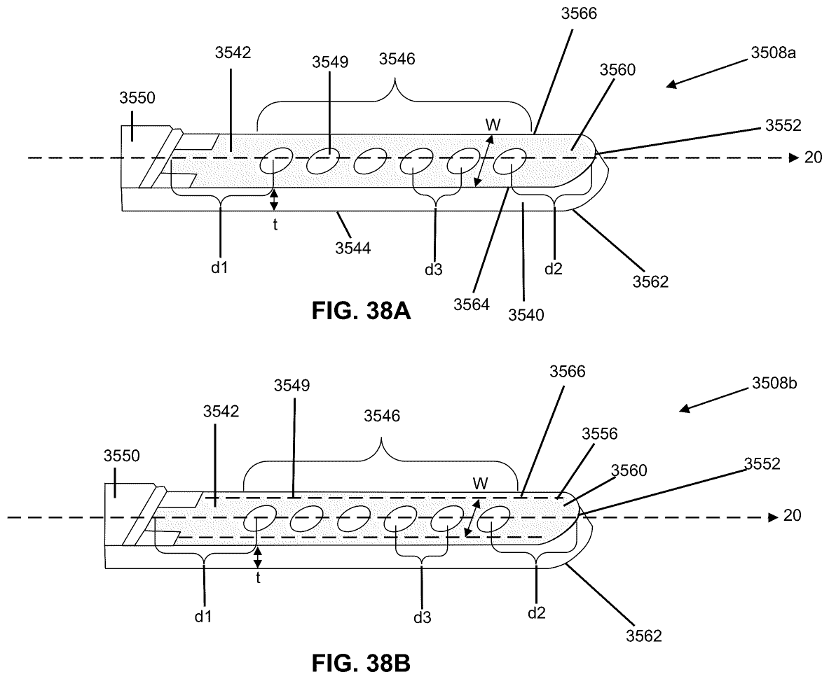

FIG. 37 shows an analyte sensor 3416 having a reduced profile tail 3508 according to an embodiment. FIGS. 37 and 38B show enlarged views of reduced profile sensor tail 3508. Analyte sensor 3416 includes a sensor tail 3508 connected to a flag 3510 by a neck 3512 as discussed above. Tail 3508 may be substantially linear and may extend along a longitudinal axis. Tail 3508 may have a width w and a thickness t. Width w of tail 3508 is a distance measured from a first side 3564 to a second side 3566 opposite first side 3564 on a first surface 3560 of analyte sensor 3416 transverse to the longitudinal axis. A thickness t of tail 3508 is a distance measured from first surface 3560 to a second surface 3562 opposite first surface 3560.

In some embodiments, tail 3508 has a reduced profile relative to a standard sensor tail, and has a width w in a range of approximately 150 μm to approximately 250 μm, approximately 170 μm to approximately 230 μm, or approximately 190 μm to approximately 210 μm. It is desirable to reduce the width of tail 3508 as much as possible to reduce foreign body response. A small width of tail 3508 may also facilitate insertion and allows for use of a smaller width insertion needle, which can minimize discomfort to the user. However, the structural properties of analyte sensor 3416 may diminish as width w decreases below 150 μm, and at smaller widths analyte sensor 3416 may not have sufficient stability or rigidity for insertion into tissue or may be susceptible to damage by bending or the like during use of analyte sensor 3416. Further, the active area 3546 on tail 3508 may require a certain minimum size to facilitate detection of the analyte and may not able to be further reduced in size without impacting detection of the analyte. Thus, tail 3508 may have a width w at least as wide as the width or diameter of active area 3546.

Width w of tail 3508 of a sensor tail can be reduced by cutting tail 3508 in longitudinal direction 20 along one or both sides 3564 and 3566 of tail 3508. Tail 3508 is cut so that side 3564 or 3566 is at or minimally spaced from an edge of active area 3546 disposed on tail 3508. In some embodiments, a distance from first or second side 3564, 3566 of tail 3508 to an edge of active area 3546 may be about 0.1 μm to 100 μm, 10 μm to 90 μm, or 25 μm to 75 μm. In some embodiments, a distance between first or second side 3564, 3566 of tail 3508 and an edge of active area 3546 is intentionally preserved in order to avoid cutting or otherwise damaging active area 3546, such as due to heat of the laser. In some embodiments, tail 3508 is cut by laser cutting. However, other cutting methods may be used.

The reduced profile tail 3508 may reduce the foreign body response when tail 3508 is positioned below skin surface. Because width w of tail 3508 is smaller compared to the tail of a sensor tail, a total surface area of tail 3508 in contact with tissue is reduced. This may help to reduce foreign body response and fibroblast build up around tail 3508. As a result, the accuracy of sensor 3416 can be maintained for a longer time in vivo.

FIGS. 38A and 38B show a sensor tail 3508a and a reduced profile sensor tail 3508b. In some embodiments, reduced profile sensor tail 3508b is manufactured by cutting from a sensor tail 3508a to reduce the width along one or both sides 3564, 3564, such as along dotted lines as shown in FIG. 38B. Sensor tail 3508 may be cut along a line in a direction of the longitudinal axis of sensor tail 3508a. Sensor tail 3508 may be cut along its full length or along a portion thereof. Sensor tail 3508 is cut to avoid damaging active area 3546. In some embodiments, cutting may be performed after the working electrode and active areas are formed.

In some embodiments, each of tail 3508a and tail 3508b has a planar substrate 3540 that includes a tip 3552. In some embodiments, tip 3552 is a rounded tip. However, in alternate embodiments, tip 3552 may be squared off, or may form a point to facilitate insertion of tail into tissue.

In some embodiments, tail 3508a, 3508b includes a substrate 3540 and a working electrode 3542 disposed on a first surface 3560 of substrate 3540. Working electrode 3542 may include a layer of a conductive material, such as carbon, among others. Tail 3508a and tail 3508b may further include a counter electrode and/or reference electrode 3544, which may be disposed on a second surface 3562 of substrate. Counter electrode and/or reference electrode 3544 may include a layer of a conductive material similar to working electrode 3542. In some embodiments, working electrode 3542 and counter electrode and/or reference electrode 3544 extend to tip 3552. In some embodiments, tail 3508a, 3508b includes a dielectric layer 3550 disposed on working electrode 3542 and counter electrode and/or reference electrode 3544 and opposite to tip 3552. In some embodiments, working electrode 3542 and counter electrode and/or reference electrode 3544 can be located on a same surface (for example, first surface 3560 or second surface 3562) of substrate 3540 with dielectric layer 3550 interposed in between such that the electrodes are electrically isolated from one another.

In some embodiments, tail 3508a, 3508b includes an active area 3546 disposed as at least one layer upon at least a portion of working electrode 3542. In some embodiments, active area 3546 can be a single spot or line of a reagent composition. In some other embodiments, active area 3546 can include a plurality of spots 3549 of a reagent composition. Spots 3549 may have a circular shape. However, in some embodiments, spots 3549 may have an oval shape, among others. Spots 3549 may be aligned along longitudinal direction 20 of tail 3508, or in other configurations, such as a grid. For example, FIGS. 38A and 38B shows an active area 3546 having six spots arranged in a line along longitudinal direction 20, FIG. 39 shows an active area 3546 having three spots arranged in a line along longitudinal direction 20, and FIG. 40 shows active area 3546 having two spots arranged in a line along longitudinal direction 20.

Each spot of active area 3546 can have a width D in a transverse direction of sensor tail 3508 in a range of approximately 100 μm to approximately 200 μm, such as approximately 120 μm to approximately 180 μm, or approximately 140 μm to approximately 160 μm. In some embodiments, a spot of active area 3546 has a circular shape, and therefore the width of the spot is a diameter D of spot 3549.

In some embodiments, a proximal portion of active area 3546, for example, a center of a spot of active area 3546 farthest from the tip, is distanced from the edge of a dielectric layer 3550 at a distance of d1. In some embodiments, the a center of a spot 3549 at a distal portion of active area 3546 closest to tip 3552 is distanced from tip 3552 of tail 3508a/3508b at a distance of d2. In some embodiments, a center of each spot 3549 of active area 3546 is distanced from a center of an adjacent spot 3549 by d3. Each of d1, d2 and d3 is measured in a longitudinal direction of tail 3508. In some embodiments, d1 is in a range of approximately 120 μm to approximately 550 μm, approximately 150 μm to approximately 500 μm, or approximately 200 μm to 450 μm. In some embodiments, d2 is in a range of approximately 110 μm to approximately 2100 μm, approximately 150 μm to approximately 2000 μm, or approximately 200 μm to 1800 μm. In some embodiments, d3 is in a range of approximately 150 μm to approximately 300 μm, approximately 160 μm to approximately 280 μm, or approximately 170 μm to 250 μm.

As shown in FIG. 39, reduced profile sensor tail 3508 has a width w transverse to longitudinal axis 20 from first side 3564 to an opposing, second side 3566. Active area 3546 includes spots 3549 having a circular shape with a diameter D. Reduced profile sensor tail 3508 can have a reduced width w, as measured from a first side 3564 to a second side 3566 opposite first side 3564 on a first surface 3560, in a range of approximately 150 μm to approximately 250 μm, such as approximately 170 μm to approximately 230 μm, or approximately 190 μm to approximately 210 μm. In some embodiments, width D of a spot of active area 3546 in a range of approximately 100 μm to approximately 200 μm, approximately 120 μm to approximately 180 μm, or approximately 140 μm to approximately 150 μm. In this way, sensor tail 3508 has reduced width relative to standard sensor tails to minimize foreign body response, and facilitate insertion into a user and minimize pain of insertion due to the smaller size, while providing sufficient space to accommodate active area 3546 without modification of active area. In some embodiments, an edge of active area 3546, such as edge of a spot 3549 is spaced from a side 3564, 3566 of tail 3508 by a distance d4 measured in a transverse direction. In some embodiments, d4 may be in a range of approximately 0.1 μm to approximately 100 μm, approximately 10 μm to approximately 90 μm, or approximately 25 μm to approximately 75 μm.

In some embodiments, a ratio of width w of tail 3508 to a width D of a spot 3549 of active area 3546 can be in a range of approximately 1:1 to approximately 1.7:1, or approximately 1.6:1 to approximately 1.1:1, or approximately 1.5:1 to approximately 1.2:1. In some embodiments, width w of reduced profile sensor tail 3508 is approximately the same as width D of a spot of active area 3546 (i.e. a ratio of 1:1), which is the smallest width w for reduced profile sensor tail 3508 without affecting (for example re-sizing or damaging) active area 3546.

A cross sectional view of a reduced profile sensor tail 3508 according to an embodiment is shown in FIG. 40. As shown in FIG. 40, tail 3508 includes a substrate 3540 with one or more electrodes arranged thereon. A working electrode 3542 is arranged on a first side of substrate 3540 and a counter or reference electrode 3544 is arranged on an opposing, second side of substrate 3540. One or more membranes may be arranged on a portion of tail 3508. A membrane 3548 is shown as overcoating at least active area 3546 of tail 3508. In certain embodiments, membrane 3548 can also overcoat some or all of working electrode 3542 and/or reference electrode 3544, or the entirety of tail 3508. One or both surfaces of substrate 3540 of tail 3508 can be overcoated with membrane 3548. Membrane 3548 can include one or more polymeric membrane materials having capabilities of limiting analyte flux to active area 3546 (i.e., membrane 3548 may be a mass transport limiting membrane having some permeability for the analyte of interest). In some embodiments, membrane 3548 can be crosslinked with a branched crosslinker in certain particular sensor configurations. For example, membrane 3548 is crosslinked with a crosslinking agent. The composition and thickness of membrane 3548 can vary to promote a desired analyte flux to active area 3546, thereby providing a desired signal intensity and stability. Tail 3508 can be operable for assaying an analyte by any of coulometric, amperometric, voltammetric, or potentiometric electrochemical detection techniques.

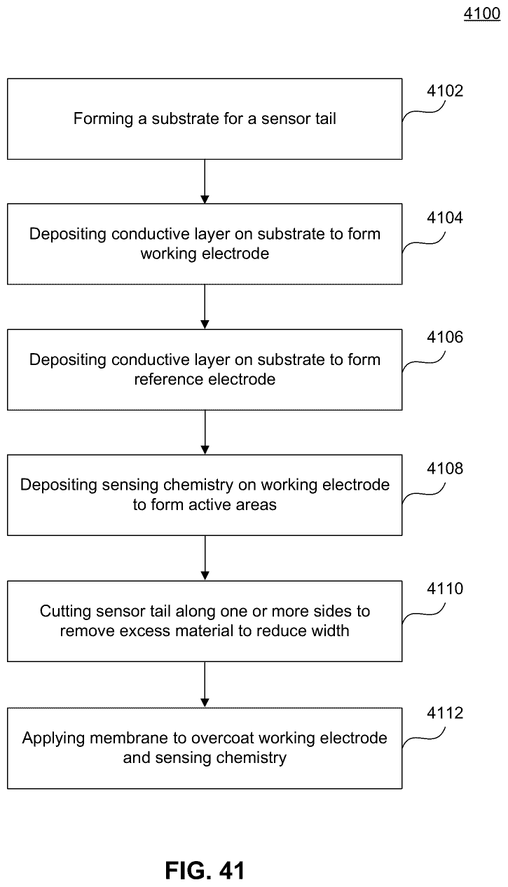

FIG. 41 shows a flow chart of an exemplary method 4100 of manufacturing a reduced profile sensor tail 3508b by removing excess material from a sensor tail 3508a (see, e.g., FIGS. 38A and 38B), thereby reducing width w. In some embodiments, method 4100 starts with step 4102, where substrate 3540 is formed from a sensor tail 3508a. As this step, substrate 3540 can have a width w for sensor tail 3508a, as measured from a first side 3564 to a second side 3566 opposite to first side 3564 of a first surface 3560, in a range of approximately 300 μm to approximately 500 μm.

At step 4104, a conductive layer is deposited onto first surface 3560 of substrate 3540 to form working electrode 3542. In some embodiments, working electrode 3542 can have the same width as substrate 3540. Optionally, at step 4106, another conductive layer is deposited onto second surface 3562 of substrate 3540 to form reference electrode 3544. In some embodiments, reference electrode 3544 can have the same width as substrate 3540. In some embodiments, instead of being deposited onto second surface 3562, dielectric layer 3550 is first deposited onto working electrode 3542 on first surface 3560, and reference electrode 3544 is deposited onto dielectric layer 3550 on first surface 3560.

At step 4108, sensing chemistry configured for detection of an analyte is disposed on working electrode 3542 to form active area 3546. Sensing chemistry may be applied in one or more spots 3549.

In order to reduce width w to manufacture reduced profile sensor tail 3508b, at step 4110, sensor tail 3508a is cut along first side 3564 and/or second side 3566 (for example, along the dotted lines in FIG. 38B) to remove excess material 3556. In some embodiments, step 4110 involves cutting along only one of first side 3564 and second side 3566, and in some other embodiments, step 4110 involves cutting along both first side 3564 and second side 3566. In some embodiments, step 4110 can be performed by laser cutting.

Removed excess material 3556 can include portions of substrate 3540, working electrode 3542, and reference electrode 3544. In some embodiments, excess material 3556 is a portion of tail 3508a anywhere from an edge of active area 3546 to first side 3564 or second side 3566 of sensor tail. In some embodiments, while it is desirable to remove as much excess material 3556 as possible to minimize the foreign body response when tail 3508b is inserted under a user's skin, excess material 3556 is removed without damaging active area 3546. In some embodiments, a distance d4 (FIG. 40) is left between first side 3564 or second side 3566 of tail 3508b and an edge of active area 3546 to avoid cutting or otherwise damaging active area 3546, such as due to heat of the laser. Because method 4100 only removes excess material 3556 from sensor tail 3508a and requires no new materials, it can be easily incorporated into existing sensor manufacturing processes.

At step 4112, membrane 3548 is applied to overcoat at least active area 3546. In some embodiments, membrane 3548 is applied to overcoat both active area 3546 and working electrode 3542 and reference electrode 3544 when present.

In is understood that a reduced profile sensor tail 3508 can be manufactured by methods other than method 4100 described above. For example, the cutting step may be performed prior to forming electrodes, or prior to forming active area. In some embodiments, membrane may be applied prior to the cutting step. In some embodiments, instead of cutting from a sensor tail 3508a, substrate 3540 is formed with a reduced width w at the first step, and then electrodes 3542, 3544, active area 3546, and/or membrane 3548 can be subsequently disposed on substrate of reduced width w sensor tail. In this way, no excess material is removed during the process.

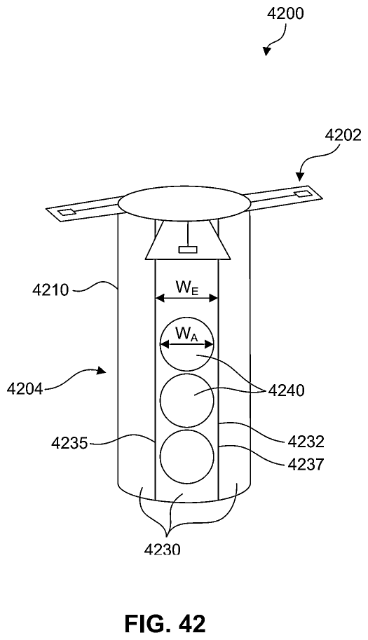

An exemplary analyte sensor having a reduced profile is shown in FIG. 42. Analyte sensor 4200 includes a substrate 4210 configured to be arranged in a tubular configuration, as discussed herein for example with respect to FIG. 5. Conductive material is disposed on a surface of substrate 4210 to form an electrode 4230. An active area 4240 may be arranged on the conductive material. The electrode 4230 may be formed to have a width W_{E} that is the same as or slightly greater than a width W_{A} of the active area 4240. The conductive material may be deposited so as to have a width that is the expected size of the active area. Alternatively, the conductive layer may be cut after the active area is deposited on the conductive layer, such as by a laser to form a boundary 4232 separating the one or more electrodes 4230. The electrode 4230 having a width that is the same or slightly greater than a size of the active area 4240 allows for reduction or minimization of the size of the electrode 4230 and reduces the exposed surface area of the conductive material. This may help to reduce or minimize a foreign body response at the electrode surface. Further, by reducing the width of one or more electrodes on the substrate, the overall width of the substrate 4210, and thus the size of the analyte sensor 4200 (e.g., a diameter of the rolled, tubular analyte sensor) may be reduced.

Analyte sensor 4200 may include a substrate 4210 having a body portion 4202 configured to be arranged above a skin surface of the user, and a tail portion 4204 configured to be arranged below the skin surface and in contact with the bodily fluid of the user for sensing analytes in the bodily fluid. The tail portion 4204 of substrate 4210 may be arranged in a tubular configuration. One or more electrodes 4230 may be arranged on the substrate 4210, specifically on tail portion 4204. A first electrode 4230 is disposed on an exterior surface of tail portion 4204 of the substrate layer 4210. Analyte sensor 4200 may have one or more active areas 4240 for detecting an analyte. Each active area 4240 include a reagent composition. Each active area 4240 may include one or more spots or drops of a reagent composition.

A first active area 4240 may be disposed on the first electrode 4230 for detecting a first analyte. A second electrode 4230 may be disposed on the exterior surface of tail portion 4204 of substrate layer 4210. A second active area 4240 may be disposed on the second electrode 4230 for detecting a second analyte, wherein the second analyte is different from the first analyte. However, in alternate embodiments, additional or fewer electrodes and active areas may be arranged on analyte sensor 4200.

The tail portion 4204 has a longitudinal axis, and each electrode 4230 has a first side 4235 opposite a second side 4237, wherein a width W_{E} of the first electrode 4230 is measured from the first side 4235 to the opposing second side 4237 in a direction transverse to the longitudinal axis. The width W_{E} of the first electrode 4230 may be in a range of approximately 150 μm to 250 μm, approximately 170 μm to approximately 230 μm, or approximately 190 μm to approximately 210 μm. A ratio of the width W_{E} of the first electrode 4230 to a width W_{A} of the first active area 4240 may be in a range of approximately 1:1 to 1.7:1, or approximately 1.6:1 to approximately 1.1:1, or approximately 1.5:1 to approximately 1.2:1. The width W_{E} of the first electrode 4230 may be substantially the same as a width W_{A} of the first active area 4240.

While the disclosed subject matter is described herein in terms of certain preferred embodiments for purpose of illustration and not limitation, those skilled in the art will recognize that various modifications and improvements can be made to the disclosed subject matter without departing from the scope thereof. Moreover, although individual features of one embodiment of the disclosed subject matter can be discussed herein or shown in the drawings of one embodiment and not in other embodiments, it should be readily apparent that individual features of one embodiment can be combined with one or more features of another embodiment or features from a plurality of embodiments.

The foregoing description of the specific embodiments will so fully reveal the general nature of the invention(s) that others can, by applying knowledge within the skill of the art, readily modify and/or adapt for various applications such specific embodiments, without undue experimentation, without departing from the general concept of the present invention(s). Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments, but should be defined only in accordance with the claims and their equivalents.

Exemplary embodiments according to a first aspect are set out in the following numbered clauses:

1. An in vivo analyte sensor for monitoring multiple analytes in a bodily fluid of a user, the in vivo analyte sensor comprising: a substrate layer arranged in a tubular configuration, wherein the substrate layer comprises a first portion configured to be arranged above the skin surface of the user, and a second portion configured to be arranged below the skin surface and in contact with the bodily fluid of the user for sensing analytes in the bodily fluid; a first electrode disposed on an exterior surface of the second portion of the substrate layer; a first reagent disposed on the first electrode for detecting a first analyte; a second electrode disposed on the exterior surface of the second portion of the substrate layer; and a second reagent disposed on the second electrode for detecting a second analyte, wherein the second analyte is different from the first analyte.

2. The analyte sensor of clause 1, wherein the first electrode comprises a conductive material disposed on the substrate layer.

3. The analyte sensor of clause 1 or 2, wherein the first electrode is electrically isolated from the second electrode.

4. The analyte sensor of any one of the preceding clauses, wherein the substrate layer comprises a non-conductive material.

5. The analyte sensor of any one of the preceding clauses, wherein a first reagent comprises one or more spots disposed on the first electrode.

6. The analyte sensor of any one of the preceding clauses, wherein the first electrode is electrically connected to a first electrical contact, wherein the first electrical contact is configured to be placed in communication with sensor electronics.

7. The analyte sensor of any one of the preceding clauses, wherein the first and second electrodes are spaced from one another in a circumferential direction of the substrate layer.

8. The analyte sensor of any one of the preceding clauses, wherein the first and second electrodes are spaced from one another in a longitudinal direction of the substrate layer.

9. The analyte sensor of any one of the preceding clauses, further comprising one or more membranes disposed on the first electrode and the first reagent.

10. The analyte sensor of any one of the preceding clauses, wherein the substrate layer comprises a core that is arranged along an interior surface of the substrate layer when the substrate layer is in the tubular configuration.

11. An analyte monitoring device for detecting multiple analytes, the analyte monitoring device comprising: a housing configured to be arranged on a skin surface of a user; sensor electronics coupled to the housing, wherein the sensor electronics comprises: one or more processors, a memory coupled to the one or more processors, and wireless communication circuitry for communicating analyte data to a receiver; and an in vivo analyte sensor, comprising: a substrate layer arranged in a tubular configuration, wherein the substrate layer comprises a first portion coupled to the sensor electronics and configured to be arranged above the skin surface of the user, and a second portion configured to be arranged below the skin surface and in contact with the bodily fluid of the user for sensing analytes in the bodily fluid; a first electrode disposed on an exterior surface of the second portion of the substrate layer; a first reagent disposed on the first electrode for detecting a first analyte; a second electrode disposed on the exterior surface of the second portion of the substrate layer; and a second reagent disposed on the second electrode for detecting a second analyte, wherein the second analyte is different from the first analyte.

12. The analyte monitoring device of clause 11, wherein the first electrode comprises a conductive material disposed on the substrate layer.

13. The analyte monitoring device of clause 11 or 12, wherein the first electrode is electrically isolated from the second electrode.

14. The analyte monitoring device of any one of clauses 11-13, wherein the substrate layer comprises a non-conductive material.

15. The analyte monitoring device of any one of clauses 11-14, wherein a plurality of spots of the first reagent are disposed on the first electrode.

16. The analyte monitoring device of any one of clauses 11-15, wherein the first electrode is electrically connected to a first electrical contact, wherein the first electrical contact is configured to be placed in communication with sensor electronics.

17. The analyte monitoring device of any one of clauses 11-16, wherein the first and second electrodes are spaced from one another in a circumferential direction of the substrate layer.

18. The analyte monitoring device of any one of clauses 11-17, wherein the first and second electrodes are spaced from one another in a longitudinal direction of the substrate layer.

19. The analyte monitoring device of any one of clauses 11-18, the in vivo analyte sensor may include one or more membranes disposed on the first electrode and the first reagent.

20. The analyte monitoring device of any one of clauses 11-19, wherein the substrate layer comprises a core that is arranged in contact with an interior surface of the substrate layer when the substrate layer is in the tubular configuration.

21. A method of manufacturing an analyte sensor for monitoring multiple analytes in a bodily fluid, the method comprising: forming a first electrode on a substrate layer; forming a second electrode on the substrate layer; depositing a first reagent on the first electrode; depositing a second reagent on the second electrode; and arranging the substrate layer in a tubular configuration.

22. The method of clause 21, wherein forming the first electrode comprises depositing a conductive material on the substrate layer.

23. The method of clause 21 or 22, further comprising encapsulating the first electrode and the first reagent in a membrane.

24. The method of any one of clauses 21-23, further comprising arranging the first electrode on the substrate layer such that the first electrode is spaced from the second electrode in a circumferential direction of the analyte sensor.

25. The method of any one of clauses 21-24, further comprising arranging the first electrode on the substrate layer such that the first electrode is spaced from the second electrode in a longitudinal direction of the analyte sensor.

26. The method of any one of clauses 21-25, further comprising forming a first electrical contact on a first extension of the substrate layer, wherein the first electrical contact is in electrical communication with the first electrode, and forming a second electrical contact on a second extension of the substrate layer, wherein the second electrical contact is in electrical communication with the second electrode.

27. The method of clause 26, further comprising folding the first extension and the second extension such that the first extension and the second extension are arranged in a plane that is transverse to a longitudinal axis of the substrate layer in the tubular configuration.

Exemplary embodiments according to a further aspect are set out in the following further set of numbered clauses.

1A. An analyte monitoring assembly for monitoring one or more analytes in a bodily fluid of a user, the analyte monitoring assembly comprising:
a housing comprising a base configured to be mounted to a body of a user;
an analyte sensor comprising a body and a plurality of sensor tails extending from the body, wherein the body is arranged on the housing and the plurality of sensor tails is configured to extend out of the housing and into the body of the user to detect the one or more analytes in the bodily fluid of the user,
wherein a first sensor tail of the plurality of sensor tails comprises a first active area for detecting signals indicative of a first analyte in the bodily fluid, and
wherein a second sensor tail of the plurality of sensor tails comprises a second active area for detecting signals indicative of a second analyte in the bodily fluid; and
sensor electronics arranged within the housing and coupled to the analyte sensor, wherein the sensor electronics is configured to receive the signals indicative of the first and second analytes from the analyte sensor and to wirelessly transmit analyte data to a receiver device.

2A. The analyte monitoring assembly of clause 1A, wherein the body of the analyte sensor is arranged perpendicularly to at least one of the plurality of sensor tails.

3A. The analyte monitoring assembly of clause 1A or 2A, wherein the body of the analyte sensor is arranged coplanar with at least one of the plurality of sensor tails.

4A. The analyte monitoring assembly of any of clauses 1A to 3A, wherein the body of the analyte sensor comprises a planar configuration.

5A. The analyte monitoring assembly of any of clauses 1A to 4A, wherein the body of the analyte sensor comprises a curvature.

6A. The analyte monitoring assembly of any of clauses 1A to 5A, wherein the housing comprises a plurality of openings through which the plurality of sensor tails extend.

7A. The analyte monitoring assembly of clause 6A, wherein the plurality of openings are arranged linearly on the base of the housing.

8A. The analyte monitoring assembly of clause 6A, wherein the plurality of openings are arranged in a ring shape on the base of the housing.

9A. The analyte monitoring assembly of any of clauses 1A to 8A, wherein each of the plurality of sensor tails comprises a length in a range of 1 mm to 4 mm.

10A. The analyte monitoring assembly of clause 9A, wherein each of the plurality of sensor tails comprises the same length.

11A. The analyte monitoring assembly of any of clauses 1A to 10A, wherein a maximum thickness of each of the plurality of sensor tails is in a range of 150 microns to 400 microns.

12A. The analyte monitoring assembly of any of clauses 1A to 11A, wherein the sensor electronics are removably coupled to the housing.

13A. The analyte monitoring assembly of any of clauses 1A to 12A, wherein at least one of the plurality of sensor tails comprises a single working electrode and an analyte-responsive enzyme.

14A. A system for monitoring a plurality of analytes in a bodily fluid of a user, the system comprising:
an analyte monitoring assembly, comprising:
   a housing comprising a base configured to be mounted to a body of a user;
   an analyte sensor comprising a body and a plurality of sensor tails extending from the body, wherein the body is arranged on the housing and the plurality of sensor tails is configured to extend out of the housing and into the body of the user to detect the one or more analytes in the bodily fluid of the user,
   wherein a first sensor tail of the plurality of sensor tails comprises a first active area for detecting signals indicative of a first analyte in the bodily fluid, and
   wherein a second sensor tail of the plurality of sensor tails comprises a second active area for detecting signals indicative of a second analyte in the bodily fluid; and
   sensor electronics arranged within the housing and coupled to the analyte sensor, wherein the sensor electronics is configured to receive the signals indicative of the first and second analytes from the analyte sensor and to wirelessly transmit analyte data to a receiver device; and
   an insertion device comprising a body and a plurality of needles extending from the body, wherein the needles are configured to pierce skin of the user to facilitate insertion of the plurality of sensor tails.

15A. The system of clause 14A, wherein each of the plurality of needles corresponds to one of the plurality of sensor tails.

16A. The system of clause 14A or 15A, wherein each of the plurality of needles has a length that is greater than a length of each of the plurality of sensor tails.

17A. The system of any of clauses 14A to 16A, wherein the body of the insertion device comprises a curvature.

18A. The system of any of clauses 14A to 17A, wherein the body of the insertion device is arranged perpendicularly to one or more of the plurality of needles.

19A. An analyte monitoring assembly for monitoring analytes in a bodily fluid of a user, the analyte monitoring assembly comprising:
a housing comprising a base configured to be mounted on a body of the user;
a first analyte sensor arranged on the housing and comprising a first body and a first plurality of sensor tails extending from the first body, wherein the first plurality of sensor tails is configured to detect one or more of the analytes in the bodily fluid;
a second analyte sensor arranged on the housing comprising a second body and a second plurality of sensor tails extending from the second body, wherein the second plurality of sensor tails is configured to detect one or more of the analytes in the bodily fluid; and
sensor electronics arranged within the housing and coupled to both the first and second analyte sensors, wherein the sensor electronics is configured to receive signals indicative of the one or more analytes from the first and second analyte sensors,
wherein the sensor electronics comprises a processor, a memory coupled to the processor, and wireless communication circuitry configured to transmit analyte data to a receiver device.

20A. The analyte monitoring assembly of clause 19A, wherein the first analyte sensor is configured to detect a first plurality of analytes and wherein the second analyte sensor is configured to detect a second plurality of analytes.

21A. The analyte monitoring assembly of clause 19A or 20A, wherein the first plurality of sensor tails and the second plurality of sensor tails extend through an opening on the base of the housing.

22A. The analyte monitoring assembly of any of clauses 19A to 21A, wherein a first sensor tail of the first plurality of sensor tails comprises a working electrode, and wherein a second sensor tail of the first plurality of sensor tails comprises a reference electrode or a counter electrode.

23A. An analyte monitoring assembly configured to detect a plurality of analytes in a bodily fluid of a user, the analyte monitoring assembly comprising,
a housing configured to be worn on a body of a user;
a plug assembly, comprising:
   a sensor support configured to be coupled to the housing,
   an analyte sensor comprising a body, a contact portion comprising one or more electrical contacts, and a plurality of sensor tails extending from the body, wherein the plurality of sensor tails are configured to detect the plurality of analytes in the bodily fluid, and wherein the analyte sensor is arranged on the sensor support, and
   an insertion device comprising a body and a plurality of needles extending from the body, wherein the insertion device is removably secured to the plug assembly; and
   sensor electronics in electrical communication with the analyte sensor to receive signals indicative of analyte levels from the analyte sensor.

24A. The analyte monitoring assembly of clause 23A, wherein each of the plurality of needles corresponds to a sensor tail of the plurality of sensor tails.

25A. The analyte monitoring assembly of clause 23A or 24A, wherein the insertion device further comprises a plurality of extensions extending from the body of the insertion device.

26A. The analyte monitoring assembly of any of clauses 23A to 25A, wherein the contact portion is arranged perpendicularly to the plurality of sensor tails.

27A. The analyte monitoring assembly of clause 26A, wherein the sensor support comprises a body defining a central opening and a platform arranged within the central opening, wherein a channel is defined between the body and the platform, and wherein the body of the analyte sensor is arranged within the channel, and the contact portion is arranged on the platform.

28A. The analyte monitoring assembly of any of clauses 23A to 27A, further comprising a connector comprising a body having a plurality of electrical contacts in electrical communication with electrical contacts of the analyte sensor.

Exemplary embodiments according to a yet further aspect are set out in the following yet further set of numbered clauses.

1B. An in vivo analyte sensor for monitoring an analyte in a bodily fluid of a user, the in vivo analyte sensor comprising:
a sensor tail, comprising:
   a substrate;
   a working electrode disposed on the substrate,
   an active area disposed on the working electrode for detecting the analyte in the bodily fluid of the user, wherein the sensor tail comprises a longitudinal axis, a first side opposite a second side, and a first surface opposite a second surface,
   wherein a width of the sensor tail is measured from the first side to the opposing second side of the sensor tail in a direction transverse to the longitudinal axis of the sensor tail, and wherein the width of the sensor tail is in a range of 150 μm to 250 μm;
   a flag comprising an electrical contact electrically connected to the working electrode; and
   a neck connecting the sensor tail and the flag.

2B. The analyte sensor of clause 1B, wherein the width of the sensor tail is in a range of 170 μm to 230 μm.

3B. The analyte sensor of clause 1B or 2B, wherein the width of the sensor tail is the same as a width of the active area.

4B. The analyte sensor of clause 1B, 2B or 3B, wherein a ratio of the width of the sensor tail to a width of the active area is in a range of 1:1 to 1.7:1.

5B. The analyte sensor of any one of clauses 1B to 4B, in which the substrate and/or the working electrode has a first side opposite a second side and a first surface opposite a second surface extending across at least a part of the width of the sensor tail, and the active area is disposed on a surface of the working electrode.

6B. The analyte sensor of any one of clauses 1B to 5B wherein a distance from the first side or the second side of the sensor tail to an edge of the active area, and/or the distance from a first side and/or a second side of the substrate to an edge of the active area, is in a range of 10 μm to 90 μm.

7B. The analyte sensor of any one of clauses 1B to 6B, wherein the active area comprises an analyte-responsive enzyme.

8B. The analyte sensor of any one of clauses 1B to 7B, wherein the active area comprises a plurality of spots of a reagent composition.

9B. The analyte sensor of any one of clauses 1B to 8B, wherein the sensor tail further comprises a membrane covering at least the active area.

10B. The analyte sensor of any one of clauses 1B to 9B, wherein the flag is arranged perpendicularly to the sensor tail.

11B. The analyte sensor of any one of clauses 1B to 10B, wherein the sensor tail further comprises a counter electrode or a reference electrode.

12B. The analyte sensor of any one of clauses 1B to 11B, wherein the sensor tail comprises a rounded tip.

13B. A sensor control device for monitoring an analyte level in a bodily fluid of a user, the sensor control device comprising:
an electronics housing;
sensor electronics arranged within the electronics housing;
an analyte sensor coupled with the sensor electronics and configured to measure an analyte level, the analyte sensor comprising:
   a sensor tail, comprising:
      a substrate;
      a working electrode disposed on the substrate,
      an active area disposed on the working electrode for detecting the analyte in the bodily fluid of the user, wherein the sensor tail comprises a longitudinal axis, a first side opposite a second side, and a first surface opposite a second surface,
      wherein a width of the sensor tail is measured from the first side to the opposing second side of the sensor tail in a direction transverse to the longitudinal axis of the sensor tail, and wherein the width of the sensor tail is in a range of 150 μm to 250 μm;
      a flag comprising an electrical contact electrically connected to the working electrode; and
      a neck connecting the sensor tail and the flag.

14B. The sensor control device of clause 13B, wherein the width of the sensor tail is in a range of 170 μm to 230 μm.

15B. The sensor control device of clause 13B or 14B, wherein the width of the sensor tail is approximately the same as a width of the active area.

16B. The sensor control device of clause 13B, 14B or 15B, wherein a ratio of the width of the sensor tail to a width of the active area is in a range of 1:1 to 1.7:1.

17B. The sensor control device of any one of clauses 13B to 16B, in which the substrate and the working electrode each have a first side opposite a second side and a first surface opposite a second surface extending across at least a part of the width of the sensor tail, and the active area is disposed on a surface of the working electrode.

18B. The sensor control device of any one of clauses 13B to 17B, wherein a distance from the first side of the substrate to an edge of the active area and/or the distance from a first side and/or a second side of the sensor tail to an edge of the active area, is in a range of 10 μm to 90 μm.

19B. The sensor control device of any one of clauses 13B to 18B, the analyte sensor further comprises a counter electrode or a reference electrode.

20B. The sensor control device of any one of clauses 13B to 19B, further comprising an adhesive patch attached to a bottom of the housing and configured to secure the sensor control device to a skin surface of a user.

21B. The sensor control device of any one of clauses 13B to 20B, further comprising a plug assembly coupled to the electronics housing and comprising a sensor module and a sharp module, wherein the sensor module comprises the analyte sensor.

22B. A method for manufacturing an analyte sensor for monitoring analyte levels, the method comprising:
forming a substrate;
depositing a conductive layer on the substrate to form a working electrode;
depositing a reagent composition on the working electrode to form an active area; and
cutting the sensor tail along a longitudinal axis of the sensor tail to reduce the width of the sensor tail to a range of 150 μm to 250 μm, wherein the width is measured in a direction transverse to the longitudinal axis of the sensor tail from a first side to an opposing second side of the sensor tail.

23B. The method of clause 22B, wherein cutting the sensor tail comprises cutting along the first side of the sensor tail in a longitudinal direction.

24B. The method of clause 23B, wherein cutting the sensor tail further comprises cutting along the second side of the sensor tail opposite the first side in the longitudinal direction.

25B. The method of clause 22B, 23B or 24B, wherein cutting the sensor tail comprises laser cutting.

26B. The method of any one of clauses 22B to 25B, further comprising applying a membrane to overcoat at least the active area of the sensor tail.

27B. The method of any one of clauses 22B to 26B, wherein the sensor tail is cut such that a ratio of the width of the sensor tail to a width of the active area is in a range of 1:1 to 1.7:1.

Exemplary embodiments according to a still further aspect are set out in the following still further numbered clauses.

1C. An analyte monitoring assembly for monitoring one or more analytes in a bodily fluid of a user, the analyte monitoring assembly comprising:
a housing configured to be mounted on a body of a user;
sensor electronics arranged within the housing; and
an analyte sensor, comprising:
   a body portion coupled to the sensor electronics and arranged within the housing,
   one or more tail portions configured to be positioned in the body of the user,
   a first electrode comprising a first active area for detecting a first analyte, and
   a second electrode comprising a second active area for detecting a second analyte.

2C. The analyte monitoring assembly of clause 1C, wherein the first electrode is connected to a first electrical contact on the body portion and the second electrode is connected to a second electrical contact on the body portion.

3C. The analyte monitoring assembly of clause 1C or 2C, wherein each of the one or more tail portions comprises a longitudinal axis extending from a proximal end of the tail portion to a distal end opposite the proximal end, wherein the first electrode comprises a first side opposite a second side in a direction traverse to the longitudinal axis, wherein a width of the first electrode measured from the first side to the second side is in a range of 150 microns to 250 microns.

4C. The analyte monitoring assembly of clause 3C, wherein a ratio of the width of the first electrode to a width of the first active area is in a range of 1:1 to 1.7:1.

5C. The analyte monitoring assembly of clause 3C, wherein the width of the first electrode is substantially the same as a width of the first active area.

6C. The analyte monitoring assembly of any of clauses 1C to 5C, wherein the first active area comprises one or more spots of a reagent composition.

7C. The analyte monitoring assembly of any of clauses 1C to 6C, wherein the one or more tail portions comprises a first tail portion comprising the first electrode and the first active area and a second tail portion comprising the second electrode and the second active area.

8C. The analyte monitoring assembly of clause 7C, wherein the first tail portion comprises a single electrode.

9C. The analyte monitoring assembly of clauses 1C to 8C, wherein an tail portion of the one or more tail portions of the analyte sensor comprises a tubular configuration.

10C. The analyte monitoring assembly of claim 9C, wherein the tail portion having the tubular configuration comprises the first electrode, the first active area, the second electrode, and the second active area.

11C. An analyte monitoring assembly for monitoring one or more analytes in a bodily fluid of a user, the analyte monitoring assembly comprising:
a housing configured to be mounted on a body of the user;
sensor electronics arranged within the housing; and
an analyte sensor comprising an body portion coupled to the sensor electronics and arranged within the housing, and a plurality of tail portions extending from the body portion, wherein each of the plurality of tail portions is configured to extend out of the housing and into the body of the user to detect the one or more analytes in the bodily fluid of the user,
wherein a first tail portion of the plurality of tail portions comprises a first active area for detecting signals indicative of a first analyte in the bodily fluid, and
wherein a second in vivotail portion of the plurality of tail portions comprises a second active area for detecting signals indicative of a second analyte in the bodily fluid.

12C. The analyte monitoring assembly of clause 11C, wherein the first tail portion comprises a longitudinal axis, a first side opposite a second side, and a first surface opposite a second surface, wherein a width of the first tail portion is measured from the first side to the opposing second side in a direction transverse to the longitudinal axis, and wherein the width is in a range of 150 μm to 250 μm.

13C. The analyte monitoring assembly of clause 11C or 12C, wherein a ratio of the width of the first tail portion to a width of the first active area is in a range of 1:1 to 1.7:1.

14C. The analyte monitoring assembly of clause 11C or 12C, wherein the width of the first tail portion is the same as a width of the first active area.

15C. The analyte monitoring assembly of any of clauses 11C to 14C, wherein the first active area comprises one or more spots of a reagent composition.

16C. An analyte monitoring assembly for monitoring multiple analytes in a bodily fluid of a user, the analyte monitoring assembly comprising:
a housing configured to be worn on a body of a user;
sensor electronics arranged within the housing;
an analyte sensor, comprising:
   a substrate layer comprising an body portion configured to be arranged above a skin surface of the user, and an tail portion configured to be arranged below the skin surface and in contact with the bodily fluid of the user for sensing analytes in the bodily fluid, wherein the tail portion comprises a tubular configuration;
   a first electrode disposed on an exterior surface of the tail portion of the substrate layer;
   a first active area disposed on the first electrode for detecting a first analyte;
   a second electrode disposed on the exterior surface of the tail portion of the substrate layer; and
   a second active area disposed on the second electrode for detecting a second analyte, wherein the second analyte is different from the first analyte.

17C. The analyte monitoring assembly of clause 16C, wherein the tail portion comprises a longitudinal axis, wherein the first electrode comprises a first side opposite a second side, wherein a width of the first electrode is measured from the first side to the opposing second side in a direction transverse to the longitudinal axis, and wherein the width is in a range of 150 μm to 250 μm.

18C. The analyte monitoring assembly of clause 17C or 18C, wherein a ratio of the width of the first electrode to a width of the first active area is in a range of 1:1 to 1.7:1.

19C. The analyte monitoring assembly of clause 17C or 18C, wherein the width of the first electrode is substantially the same as a width of the first active area.

20C. The analyte monitoring assembly of any of clauses 16C to 19C, wherein the first active area comprises one or more spots of a reagent composition.

What is claimed is:

1. An analyte monitoring assembly for monitoring one or more analytes in a bodily fluid of a user, the analyte monitoring assembly comprising:
a housing configured to be mounted on a body of a user;
sensor electronics arranged within the housing; and
an analyte sensor, comprising:
a body portion coupled to the sensor electronics and arranged within the housing,
one or more tail portions configured to be positioned in the body of the user,
a first electrode comprising a first active area for detecting a first analyte, and
a second electrode comprising a second active area for detecting a second analyte.

2. The analyte monitoring assembly of claim 1, wherein the first electrode is connected to a first electrical contact on the body portion and the second electrode is connected to a second electrical contact on the body portion.

3. The analyte monitoring assembly of claim 1, wherein each of the one or more tail portions comprises a longitudinal axis extending from a proximal end of the tail portion to a distal end opposite the proximal end, wherein the first electrode comprises a first side opposite a second side in a direction traverse to the longitudinal axis, wherein a width of the first electrode measured from the first side to the second side is in a range of 150 microns to 250 microns.

4. The analyte monitoring assembly of claim 3, wherein a ratio of the width of the first electrode to a width of the first active area is in a range of 1:1 to 1.7:1.

5. The analyte monitoring assembly of claim 3, wherein the width of the first electrode is substantially the same as a width of the first active area.

6. The analyte monitoring assembly of claim 1, wherein the first active area comprises one or more spots of a reagent composition.

7. The analyte monitoring assembly of claim 1, wherein the one or more tail portions comprises a first tail portion comprising the first electrode and the first active area and a second tail portion comprising the second electrode and the second active area.

8. The analyte monitoring assembly of claim 7, wherein the first tail portion comprises a single electrode.

9. The analyte monitoring assembly of claim 1, wherein a tail portion of the one or more tail portions of the analyte sensor comprises a tubular configuration.

10. The analyte monitoring assembly of claim 9, wherein the tail portion having the tubular configuration comprises the first electrode, the first active area, the second electrode, and the second active area.

11. An analyte monitoring assembly for monitoring one or more analytes in a bodily fluid of a user, the analyte monitoring assembly comprising:
a housing configured to be mounted on a body of the user;
sensor electronics arranged within the housing; and
an analyte sensor comprising a body portion coupled to the sensor electronics and arranged within the housing, and a plurality of tail portions extending from the body portion, wherein each of the plurality of tail portions is configured to extend out of the housing and into the body of the user to detect the one or more analytes in the bodily fluid of the user,
wherein a first tail portion of the plurality of tail portions comprises a first active area for detecting signals indicative of a first analyte in the bodily fluid, and
wherein a second tail portion of the plurality of tail portions comprises a second active area for detecting signals indicative of a second analyte in the bodily fluid.

12. The analyte monitoring assembly of claim 11, wherein the first tail portion comprises a longitudinal axis, a first side opposite a second side, and a first surface opposite a second surface, wherein a width of the first tail portion is measured from the first side to the opposing second side in a direction transverse to the longitudinal axis, and wherein the width is in a range of 150 μm to 250 μm.

13. The analyte monitoring assembly of claim 12, wherein a ratio of the width of the first tail portion to a width of the first active area is in a range of 1:1 to 1.7:1.

14. The analyte monitoring assembly of claim 12, wherein the width of the first tail portion is the same as a width of the first active area.

15. The analyte monitoring assembly of claim 11, wherein the first active area comprises one or more spots of a reagent composition.

16. An analyte monitoring assembly for monitoring multiple analytes in a bodily fluid of a user, the analyte monitoring assembly comprising:
a housing configured to be worn on a body of a user;
sensor electronics arranged within the housing;
an analyte sensor, comprising:
a substrate layer comprising a body portion configured to be arranged above a skin surface of the user, and a tail portion configured to be arranged below the skin surface and in contact with the bodily fluid of the user for sensing analytes in the bodily fluid, wherein the tail portion comprises a tubular configuration;
a first electrode disposed on an exterior surface of the tail portion of the substrate layer;
a first active area disposed on the first electrode for detecting a first analyte;
a second electrode disposed on the exterior surface of the tail portion of the substrate layer; and
a second active area disposed on the second electrode for detecting a second analyte, wherein the second analyte is different from the first analyte.

17. The analyte monitoring assembly of claim 16, wherein the tail portion comprises a longitudinal axis, wherein the first electrode comprises a first side opposite a second side, wherein a width of the first electrode is measured from the first side to the opposing second side in a direction transverse to the longitudinal axis, and wherein the width is in a range of 150 μm to 250 μm.

18. The analyte monitoring assembly of claim 17, wherein a ratio of the width of the first electrode to a width of the first active area is in a range of 1:1 to 1.7:1.

19. The analyte monitoring assembly of claim 17, wherein the width of the first electrode is substantially the same as a width of the first active area.

20. The analyte monitoring assembly of claim 16, wherein the first active area comprises one or more spots of a reagent composition.
